(19) United States
(12) Patent Application Publication
(10) Pub. No.: US 2021/0393740 A1
(43) Pub. Date: Dec. 23, 2021

(54) PTPRS AND PROTEOGLYCANS IN RHEUMATOID ARTHRITIS

(71) Applicants: La Jolla Institute for Immunology, La Jolla, CA (US); The Regents of the University of California, Oakland, CA (US)

(72) Inventor: Nunzio Bottini, La Jolla, CA (US)

(21) Appl. No.: 17/278,200

(22) PCT Filed: Sep. 19, 2019
(86) PCT No.: PCT/US2019/052028

§ 371 Date: Mar. 19, 2021

Related U.S. Application Data
(60) Provisional application No. 62/733,545, filed on Sep. 19, 2018.

(51) Int. Cl. A61K 38/17; A61K 39/395; A61K 38/46; A61P 19/02; A61P 37/06

(52) U.S. Cl. CPC A61K 38/1793; A61K 39/3955; A61P 37/06; A61P 19/02; A61K 38/465

(57) ABSTRACT

Provided herein, inter alia, are pharmaceutical compositions methods thereof that include a first amount of a PTPRS de-clustering agent and a second amount of a TNF inhibitor or an IL-6 inhibitor in synergistic amounts. The synergistic combinations provide (a) amelioration of disease or one or more symptoms of disease or (b) delay of onset of disease or one or more symptoms of disease.

### CROSS-REFERENCES TO RELATED APPLICATIONS

This application claims priority to U.S. Provisional Application No. 62/733,545, filed Sep. 19, 2018, which is incorporated by reference herein in its entirety.

### STATEMENT AS TO RIGHTS TO INVENTIONS MADE UNDER FEDERALLY SPONSORED RESEARCH AND DEVELOPMENT

This invention was made with Government support under grant number R01 AR066053 awarded by the National Institutes of Health. The Government has certain rights in this invention.

### REFERENCE TO A SEQUENCE LISTING, A TABLE OR A COMPUTER PROGRAM LISTING APPENDIX SUBMITTED AS AN ASCII TEXT FILE

Sequence Listing written in file 048513-510001WO_SEQUENCE_LISTING_ST25.txt created on Sep. 19, 2019, 76,475 bytes, machine format IBM-PC, MS Windows operating system, is hereby incorporated by reference.

### BACKGROUND OF THE INVENTION

Approximately 5 to 8% of people in the United States suffer from an autoimmune disease. Researchers have identified more than 80 different autoimmune diseases and suspect that many more diseases may have an autoimmune component. Rheumatoid arthritis (RA) alone afflicts roughly 2.5 million people in the United States RA affects the joints and bones but may also affect different organs and biological systems.

Fibroblast-like synoviocytes (FLS) are key players in mediating inflammation and joint destruction in rheumatoid arthritis (RA). There is an increased level of attention to this cell type as the possible target of a new generation of anti-RA therapies, which would be used in combination with immunomodulators to help control disease without increasing immune-suppression. The behavior of FLS is controlled by multiple interconnected signal transduction pathways. Several of these pathways involve reversible phosphorylation of proteins on tyrosine residues, which is the result of the balanced action of protein tyrosine kinases (PTKs) and phosphatases (PTPs). PTKs are mediators of FLS growth and invasiveness. PTPs act by removing phosphate groups from phosphorylated tyrosine residues on proteins. Receptor protein tyrosine phosphatases (RPTPs or PTPRs) are PTPs that generally have a variable length extracellular domain followed by a transmembrane region and a C-terminal catalytic cytoplasmic domain.

Despite the availability of immunosuppressive disease-modifying anti-rheumatic agents (DMARDs), many rheumatoid arthritis (RA) patients still fail to achieve remission. Fibroblast-like synoviocytes (FLS) are non-immunological joint-lining cells that become invasive during RA. Non-immunosuppressive agents targeting FLS in combination with DMARDs have the potential to improve control of RA without enhancing the risk of infections. We recently reported that disrupting the interaction between the receptor protein tyrosine phosphatase sigma (RPTPσ) and the proteoglycan syndecan-4 using an RPTPσ decoy biologic (RPTPσ-Ig1&2) reduces FLS cartilage invasion.

Current therapies for chronic inflammatory conditions such as RA include administration of tumor necrosis factor-α (TNF) or interleukin-6 (IL-6) inhibitors. However, such therapies include significant side effects and efficacy could be improved.

### BRIEF SUMMARY OF THE INVENTION

Provided herein, inter alia, are pharmaceutical compositions that include a first amount of a PTPRS de-clustering agent and a second amount of a TNF inhibitor, wherein the second amount is below a therapeutically effective level of the TNF inhibitor. In some embodiments, the therapeutically effective level of the TNF inhibitor is measured by an increase in (a) amelioration of disease or one or more symptoms of disease or (b) delay of onset of disease or one or more symptoms of disease.

In some embodiments, the TNF inhibitor is Etanercept. In some embodiments, the second amount is below 50 mg Etanercept. In some embodiments, the TNF inhibitor is Adalimumab. In some embodiments, the second amount is below 40 mg Adalimumab. In some embodiments, the TNF inhibitor is Infliximab. In some embodiments, the second amount is below 3 mg/kg Infliximab. In some embodiments, the TNF inhibitor is Golimumab. In some embodiments, the second amount is below 50 mg Golimumab. In some embodiments, the TNF inhibitor is Certolizumab or Certolizumab pegol. In some embodiments, the second amount is below 200 mg Certolizumab or Certolizumab pegol.

Provided herein are pharmaceutical compositions that include a first amount of a PTPRS de-clustering agent and a second amount of an IL-6 inhibitor, wherein the second amount is below a therapeutically effective level of the IL-6 inhibitor. In some embodiments, the IL-6 inhibitor is Tocilizumab, or Atlizumab. In some embodiments, the drug is administered by IV infusion and the second amount is below 4 mg/kg Tocilizumab, or Atlizumab. In some embodiments, the drug is administered SC, and the second amount is below 162 mg Tocilizumab, or Atlizumab. In some embodiments, the IL-6 inhibitor is Sarilumab, or Kevzara. In some embodiments, the second amount is below 100 mg Sarilumab, or Kevzara.

Provided herein are any of the above pharmaceutical compositions, wherein the first amount is below a therapeutically effective level of the PTPRS de-clustering agent. In some embodiments, the PTPRS de-clustering agent includes one or both of PTPRS immunoglobulin-like domain 1 (Ig1) and immunoglobulin-like domain 2 (Ig2). In some embodiments, the PTPRS de-clustering agent binds heparan sulfate. In some embodiments, the PTPRS de-clustering agent lacks a transmembrane domain. In some embodiments, the PTPRS de-clustering agent lacks an intracellular domain.

In some embodiments, provided herein are pharmaceutical compositions of any one of the above combinations of a first amount of PTPRS de-clustering agent and a second amount of a TNF or an IL-6 inhibitor are present in a combined synergistic amount.

Provided herein are methods of treating an autoimmune disease in a subject by administering to the subject the pharmaceutical compostions of any of the combinations of PTPRS de-clustering agent and TNF inhibitor or PTPRS de-clustering agent and IL-6 inhibitor described above. In some embodiments, the PTPRS de-clustering agent is not chondroitin sulfate. The autoimmune disease can be arthritis and can be rheumatoid arthritis. The autoimmune disease can be scleroderma or Crohn's disease. The method can include a dosing schedule for which the pharmaceutical composition is administered.

Provided herein are methods of decreasing fibroblast activity in a subject by administering any of the combinations of drugs described above. In some embodiments, the PTPRS de-clustering agent is not chondroitin sulfate. The fibroblast activity can be fibroblast migration. The fibroblast activity can be collagen production, glycosaminoglycan production, reticular and elastic fiber production, cytokine production, chemokine production, glycoprotein production or combinations thereof. The fibroblast activity can be extracellular matrix production. The fibroblasts can be synovial fibroblasts, dermal fibroblasts, or interstitial fibroblasts. In some embodiments, the subject has a fibroblast-mediated disease. The fibroblast-mediated disease can be fibrosis. The fibrosis can be pulmonary fibrosis, idiopathic pulmonary fibrosis, liver fibrosis, endomyocardial fibrosis, atrial fibrosis, mediastinal fibrosis, myelofibrosis, retroperitoneal fibrosis, nephrogenic systemic fibrosis, skin fibrosis, or arthrofibrosis. The fibroblast-mediated disease can be a fibroblast-mediated autoimmune disease. The fibroblast-mediated autoimmune disease can be Crohn's disease, arthritis, rheumatoid arthritis, or scleroderma.

Provided herein are methods of modulating extracellular matrix in a subject by administering to the subject an effective amount of any of the pharmaceutical compostions disclosed above, wherein administration modulates the extracellular matrix in the subject. The modulation of the extracellular matrix can be modulation of one or more components of the extracellular matrix. The subject can have an extracellular matrix disease.

### BRIEF DESCRIPTION OF THE DRAWINGS

FIG. 1 show PTPRS (encoding RPTPσ) expression in RA and osteoarthritis (OA) FLS. FIG. 1A shows relative PTPRS expression in RA (n=3) and OA (n=3) FLS, with or without TNF. FIG. 1B shows relative PTPRS expression pool in RA and OA (n=3).

FIG. 2 show TNF-induced RPTPσ expression in mouse FLS; mouse FLS were serum-starved for 24 hours and then stimulated with 50 ng/ml of TNF or unstimulated for 24 hours. FIG. 2A shows a western blot showing RA FLS RPTPσ protein expression with or without TNF stimulation. FIG. 2B shows the mouse FLS RPTPσ mRNA relative expression, with or without TNF stimulation.

FIG. 3 shows the amino acid sequence of the linker regions of Ig1&2His- and Fc-constructs.

FIG. 4 show results of scratch assay with Fc-Ig1&2 Construct 1, on serum-starved FLS (n=3) monolayers in absence or presence of TNF or PTPRS Fc-Ig1&2 (Ig1&1), alone or in combination. FIG. 4A shows wound width (in arbitrary units) in RA 1757 at the time of wounding (0 hours), or at 12, 24, or 48 hours after wounding, in the presence or absence of TNF or Ig1&2, alone or in combination. FIG. 4B shows wound with (in arbitrary units) in RA 1775 at the time of wounding (0 hours), or at 12, 24, or 48 hours after wounding, in the presence or absence of TNF or Ig1&2, alone or in combination. FIG. 4C shows wound width (in arbitrary units) in RA 1402 at the time of wounding (0 hours), or at 12, 24, or 48 hours after wounding, in the presence or absence of TNF or Ig1&2, alone or in combination. FIG. 4D shows wound width (in arbitrary units) in RA FLS at the time of wounding (0 hours), or at 12, 24, or 48 hours after wounding, in the presence or absence of TNF or Ig1&2, alone or in combination. Data were analyzed using two-way analysis of variance (ANOVA, ****, P<0.0001).

FIG. 5 show RPTPσ expression in monocytes or macrophages from arthritic K/B×N serum transfer induced arthritis (STIA) mice. FIG. 5A shows RPTPσ expression in classical (Ly6C+CD43-), intermediate (Ly6C+CD43+), or non-classical (Ly6C-CD43+) circulating monocytes (blood monocytes); the expression of RPTPσ in plasmacytoid dendritic cells (pDC) is shown for comparison. FIG. 5B shows RPTPσ expression in (Ly6C+CD43-), intermediate (Ly6C+CD43+), or non-classical (Ly6C-CD43+) joint macrophages (ankle macrophages); the expression of RPTPσ in plasmacytoid dendritic cells (pDC) which are known to express high levels of RPTPσ is shown for comparison.

FIG. 6 show that STIA in mice reconstituted with bone marrow from RPTPσ-Knockout (KO) versus wild type (WT) mice (the His-tagged Ig1&2 construct was utilized) showed the same responsiveness to Ig1&2 in the STIA model. FIG. 6A shows clinical scoring of either WT or RPTPσ-KO STIA mice treated with the Ig1&2 “in use” construct on days 0, 2, 4, 6, and 8, after receiving injection of Ig1&2 or of vehicle. FIG. 6B shows the change of ankle thickness (in mm) of either WT or RPTPσ-KO STIA mice treated with the Ig1&2 “in use” construct on days 0, 2, 4, 6, and 8, after receiving injection of Ig1&2 or of vehicle. Means+/−s.e.m. are shown, and data were analyzed using two-way analysis of variance (ANOVA, **, P<0.01).

FIG. 7 shows that Fc-Ig1&2 “in use” was effective at reversing collagen-induced arthritis (CIA) as monotherapy or in combination with TNF inhibitor mEtanercept (p75mTNFr:Fc), and that the combination of Ig1&2 and mEtanercept showed significantly higher efficacy than TNF inhibitor alone. Means+s.e.m. are shown.

FIG. 8 shows that titration of TNF inhibitor mEtanercept (p75mTNFr:Fc) demonstrated effect below the therapeutically effective level (2 mg/kg) and the induction of PTPRS in joint homogenates of CIA mice. FIG. 8A shows the evolution of the clinical score for mice that received primary immunization at day 0 and were boosted at day 21. Means+s.e.m. are shown. FIG. 8B shows relative expression of PTPRS in ankles of mice treated with vehicle, or different dosages of mEtanercept.

FIG. 9 shows clinical scoring for arthritis of mice treated with Fc-Ig1&2 “Construct 1” or Etanercept, alone or in combination.

FIG. 10 shows anti-collagen antibody levels after first and second administration of Incomplete Freund's Adjuvant (IFA) in mice treated with Fc-Ig1&2 “Construct 1”.

FIG. 11 shows the therapeutic synergy between Fc-Ig1&2 and mEtanercept in CIA. FIG. 11A shows a cartoon summary of the experiment and a time course of clinical score post treatment of Fc-Ig1&2 and mEtanercept separately and together. FIG. 11B shows the levels of anti type II collagen IgG antibodies in the serum of mice of FIG. 11A, at the end of the experiment. FIG. 11C shows the histopathological scores for synovitis in mice treated as shown in FIG. 11A. FIG. 11D shows the histopathological scores for bone erosion in mice treated as shown in FIG. 11A. FIG. 11E shows the histopathological scores for cartilage depletion in mice treated as shown in FIG. 11A. In FIGS. 11B-E, the graphs show mean±s.e.m., *P<0.05, **P<0.01, ***P<0.001, ****P<0.0001, by two-way ANOVA (FIG. 11A vs Vehicle group) or one-way ANOVA (FIGS. 11C-E).

FIG. 12 shows RNA-seq data from RA FLS isolated from the synovial tissue of RA patients.

FIG. 13 show the efficacy of Fc-Ig1&2 in experimental mouse arthritis. FIG. 13A shows reduction in ankle thickness upon treatment in mouse model. FIG. 13B shows radiographs of mice with established STIA injected with 111-indium (¹¹¹In)-labeled Fc-Ig1&2.

FIG. 13C shows densitometric measurements of radiographs. FIG. 13D shows effect of Fc-Ig1&2 and mEtanercept administration to arthritic CIA mice. FIG. 13E shows the effect of Fc-Ig1&2 and mEtanercept on production of anti-collagen antibodies. FIG. 13F-G shows the effect of Fc-Ig1&2 administration on various clinical measures.

FIG. 14 shows the effect of Ig1&2 (His-Ig1&2 and Fc-Ig1&2) on various clinical measures. FIG. 14A shows effect of Ig1&2 on STIA in CD45.1 congenic mice subjected to lethal irradiation (>1000 Rad) and bone-marrow transplantation from CD45.2 WT or PTPRS KO mice. FIG. 14B shows Ig1&2 to arthritic K/B×N transgenic mice which develop spontaneous arthritis starting at 6-7 weeks of age. FIG. 14C shows the effect of Ig1&2 on pDC depleted mice prior to subjecting mice to STIA. FIG. 14D shows the effect of Fc-Ig1&2 on accumulation or expansion of regulatory T cells (Tregs) or Th17 cells in arthritic ankles of CIA mice. FIG. 14E shows the effect of Fc-Ig1&2 on the number and frequency of MHCII⁺CD64⁺ and MHCII⁻CD64⁺ macrophages in the same ankles. FIG. 14F shows the effect of Fc-Ig1&2 on overall titers of anti-collagen IgG antibodies and the titers of anti-collagen IgG subclasses IgG1, IgG2a, IgG2b and IgG3. FIGS. 14G-H show the effect of Fc-Ig1&2 on the frequency and numbers of Tfh cells and GC B cells, respectively. FIG. 14I-J shows the effect of Ig1&2 on the numbers of Th1, Th17 or Tregs in lymph nodes.

FIG. 15 show the effect of TNF on PTPRS expression. FIG. 15A shows the epigenomic landscape of the PTPRS locus in RA FLS with six histone modifications, open chromatin (ATAC-seq), RNA-Seq and DNA methylation. FIG. 15B shows the effect of siRNA-mediated knock-down of USF2 on PTPRS in RA FLS. FIG. 15C shows ChIP assay of USF2 binding to the promoter region of PTPRS. FIG. 15D shows a PTPRS luciferase reporter assay in the presence and absence of USF2.

FIG. 16 show the effect of TNF on PTPRS expression. FIG. 16A shows the effect of increasing amounts of TNF on PTPRS expression in RA. FIG. 16B shows the effect of increasing amounts of TNF on PTPRS expression in OA. FIG. 16C shows a comparison of the basal level of expression of PTPRS between RA and OA FLS.

FIG. 17 shows in vitro motility assays. FIG. 17A shows the change in would area in the absence of TNF. FIG. 17B shows the change in would area in the presence of 50 ng/ml TNF.

FIG. 18 shows the effect of subtherapeutic (0.1 and 0.25 mg) Ig1&2 administered as monotherapy and in combination with a subtherapeutic (2 mg/kg) dose of TNF inhibitor in reversal of mouse collagen-induced arthritis (CIA). CIA mice were treated with human IgG1 Fc control, Vehicle (N=9), 2 mg/kg murine etanercept (mEtan, N=9), 0.5 mg Fc-Ig1&2 (N=10), 0.25 mg Fc-Ig1&2 (N=10), 0.1 mg Fc-Ig1&2 (N=10), 2 mg/kg mEtan+0.1 mg Fc-Ig1&2 (combo, N=10) by intraperitoneal injections on days 44, 46 and 48 post primary immunization. Arthritis was assessed every 2 days by clinical scoring.

FIG. 19 shows relative PTPRS expression in RA FLS, with or without IL-6.

FIG. 20 shows the effect of Fc-Ig1&2 on arthritic scores in the STIA mouse model. From top to bottom, at day 14, and for each graph, the lines shown on FIG. 20 respectively represent the following: vehicle, 0.05 mg Fc-Ig1&2, 0.1 mg Fc-Ig1&2, 025 mg Fc-1&2, and 0.5 mg Fc-1&2.

### DETAILED DESCRIPTION OF THE INVENTION

The present application relates to pharmaceutical compositions and methods of treatment relating thereto that include combinations of a PTPRS de-clustering agent and a TNF inhibitor or an IL-6 inhibitor, in amounts that are below the therapeutically effective level of the agent or inhibitor alone. Applicant found surprising synergy between PTPRS de-clustering agents and TNF inhibitors or PTPRS de-clustering agents and IL-6 inhibitors. While not wishing to be limited by theory, PTPRS de-clustering agents can be used for at least the following instances: (1) as an adjuvant in partial responders or (2) as an immunosuppressant sparing agent for patients who are experiencing unwanted infections or for patients who have good control but want to reduce their risk of infection.

The terms “subject,” “patient,” “individual,” etc. are not intended to be limiting and can be generally interchanged. That is, an individual described as a “patient” does not necessarily have a given disease, but may be merely seeking medical advice.

A “control” or “standard control” refers to a sample, measurement, or value that serves as a reference, usually a known reference, for comparison to a test sample, measurement, or value. For example, a test sample can be taken from a patient suspected of having a given disease (e.g. an autoimmune disease, inflammatory autoimmune disease, cancer, infectious disease, immune disease, or other disease) and compared to a known normal (non-diseased) individual (e.g. a standard control subject). A standard control can also represent an average measurement or value gathered from a population of similar individuals (e.g. standard control subjects) that do not have a given disease (i.e. standard control population), e.g., healthy individuals with a similar medical background, same age, weight, etc. A standard control value can also be obtained from the same individual, e.g. from an earlier-obtained sample from the patient prior to disease onset. One of skill will recognize that standard controls can be designed for assessment of any number of parameters (e.g. RNA levels, protein levels, specific cell types, specific bodily fluids, specific tissues, synoviocytes, synovial fluid, synovial tissue, fibroblast-like synoviocytes, macrophage-like synoviocytes, etc).

One of skill in the art will understand which standard controls are most appropriate in a given situation and be able to analyze data based on comparisons to standard control values. Standard controls are also valuable for determining the significance (e.g. statistical significance) of data. For example, if values for a given parameter are widely variant in standard controls, variation in test samples will not be considered as significant.

The terms “dose” and “dosage” are used interchangeably herein. A dose refers to the amount of active ingredient given to an individual at each administration. The dose will vary depending on a number of factors, including the range of normal doses for a given therapy, frequency of administration; size and tolerance of the individual; severity of the condition; risk of side effects; and the route of administration. One of skill will recognize that the dose can be modified depending on the above factors or based on therapeutic progress. The term “dosage form” refers to the particular format of the pharmaceutical or pharmaceutical composition, and depends on the route of administration. For example, a dosage form can be in a liquid form for nebulization, e.g., for inhalants, in a tablet or liquid, e.g., for oral delivery, or a saline solution, e.g., for injection.

As used herein, the terms “treat” and “prevent” may refer to any delay in onset, reduction in the frequency or severity of symptoms, amelioration of symptoms, improvement in patient comfort or function (e.g. joint function), decrease in severity of the disease state, etc. The effect of treatment can be compared to an individual or pool of individuals not receiving a given treatment, or to the same patient prior to, or after cessation of, treatment. The term “prevent” generally refers to a decrease in the occurrence of a given disease (e.g. an autoimmune, inflammatory autoimmune, cancer, infectious, immune, or other disease) or disease symptoms in a patient. As indicated above, the prevention may be complete (no detectable symptoms) or partial, such that fewer symptoms are observed than would likely occur absent treatment.

By “therapeutically effective dose or amount” as used herein is meant a dose that produces effects for which it is administered (e.g. treating or preventing a disease). The exact dose and formulation will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Remington: The Science and Practice of Pharmacy, 20th Edition, Gennaro, Editor (2003), and Pickar, Dosage Calculations (1999)). For example, for the given parameter, a therapeutically effective amount will show an increase or decrease of at least 5%, 10%, 15%, 20%, 25%, 40%, 50%, 60%, 75%, 80%, 90%, or at least 100%. Therapeutic efficacy can also be expressed as “-fold” increase or decrease. For example, a therapeutically effective amount can have at least a 1.2-fold, 1.5-fold, 2-fold, 5-fold, or more effect over a standard control. A therapeutically effective dose or amount may ameliorate one or more symptoms of a disease. A therapeutically effective dose or amount may prevent or delay the onset of a disease or one or more symptoms of a disease when the effect for which it is being administered is to treat a person who is at risk of developing the disease.

The term “diagnosis” refers to a relative probability that a disease (e.g. an autoimmune, inflammatory autoimmune, cancer, infectious, immune, or other disease) is present in the subject. Similarly, the term “prognosis” refers to a relative probability that a certain future outcome may occur in the subject with respect to a disease state. For example, in the context of the present invention, prognosis can refer to the likelihood that an individual will develop a disease (e.g. an autoimmune, inflammatory autoimmune, cancer, infectious, immune, or other disease), or the likely severity of the disease (e.g., duration of disease). The terms are not intended to be absolute, as will be appreciated by any one of skill in the field of medical diagnostics.

“Nucleic acid” or “oligonucleotide” or “polynucleotide” or grammatical equivalents used herein means at least two nucleotides covalently linked together. The term “nucleic acid” includes single-, double-, or multiple-stranded DNA, RNA and analogs (derivatives) thereof. Oligonucleotides are typically from about 5, 6, 7, 8, 9, 10, 12, 15, 25, 30, 40, 50 or more nucleotides in length, up to about 100 nucleotides in length. Nucleic acids and polynucleotides are a polymers of any length, including longer lengths, e.g., 200, 300, 500, 1000, 2000, 3000, 5000, 7000, 10,000, etc. In certain embodiments. the nucleic acids herein contain phosphodiester bonds. In other embodiments, nucleic acid analogs are included that may have alternate backbones, comprising, e.g., phosphoramidate, phosphorothioate, phosphorodithioate, or O-methylphosphoroamidite linkages (see Eckstein, Oligonucleotides and Analogues: A Practical Approach, Oxford University Press); and peptide nucleic acid backbones and linkages. Other analog nucleic acids include those with positive backbones; non-ionic backbones, and nonribose backbones, including those described in U.S. Pat. Nos. 5,235,033 and 5,034,506, and Chapters 6 and 7, ASC Symposium Series 580, Carbohydrate Modifications in Antisense Research, Sanghui & Cook, eds. Nucleic acids containing one or more carbocyclic sugars are also included within one definition of nucleic acids. Modifications of the ribose-phosphate backbone may be done for a variety of reasons, e.g., to increase the stability and half-life of such molecules in physiological environments or as probes on a biochip. Mixtures of naturally occurring nucleic acids and analogs can be made; alternatively, mixtures of different nucleic acid analogs, and mixtures of naturally occurring nucleic acids and analogs may be made.

A particular nucleic acid sequence also encompasses “splice variants.” Similarly, a particular protein encoded by a nucleic acid encompasses any protein encoded by a splice variant of that nucleic acid. “Splice variants,” as the name suggests, are products of alternative splicing of a gene. After transcription, an initial nucleic acid transcript may be spliced such that different (alternate) nucleic acid splice products encode different polypeptides. Mechanisms for the production of splice variants vary, but include alternate splicing of exons. Alternate polypeptides derived from the same nucleic acid by read-through transcription are also encompassed by this definition. Any products of a splicing reaction, including recombinant forms of the splice products, are included in this definition. An example of potassium channel splice variants is discussed in Leicher, et al., J. Biol. Chem. 273(52):35095-35101 (1998).

Nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are near each other, and, in the case of a secretory leader, contiguous and in reading phase. However, enhancers do not have to be contiguous. Linking is accomplished by ligation at convenient restriction sites. If such sites do not exist, the synthetic oligonucleotide adaptors or linkers are used in accordance with conventional practice.

The term “probe” or “primer”, as used herein, is defined to be one or more nucleic acid fragments whose specific hybridization to a sample can be detected. A probe or primer can be of any length depending on the particular technique it will be used for. For example, PCR primers are generally between 10 and 40 nucleotides in length, while nucleic acid probes for, e.g., a Southern blot, can be more than a hundred nucleotides in length. The probe may be unlabeled or labeled as described below so that its binding to the target or sample can be detected. The probe can be produced from a source of nucleic acids from one or more particular (preselected) portions of a chromosome, e.g., one or more clones, an isolated whole chromosome or chromosome fragment, or a collection of polymerase chain reaction (PCR) amplification products. The length and complexity of the nucleic acid fixed onto the target element is not critical to the invention. One of skill can adjust these factors to provide optimum hybridization and signal production for a given hybridization procedure, and to provide the required resolution among different genes or genomic locations.

The probe may also be isolated nucleic acids immobilized on a solid surface (e.g., nitrocellulose, glass, quartz, fused silica slides), as in an array. In some embodiments, the probe may be a member of an array of nucleic acids as described, for instance, in WO 96/17958. Techniques capable of producing high density arrays can also be used for this purpose (see, e.g., Fodor (1991) Science 767-773; Johnston (1998) Curr. Biol. 8: R171-R174; Schummer (1997) Biotechniques 23: 1087-1092; Kern (1997) Biotechniques 23: 120-124; U.S. Pat. No. 5,143,854).

A “labeled nucleic acid probe or oligonucleotide” is one that is bound, either covalently, through a linker or a chemical bond, or noncovalently, through ionic, van der Waals, electrostatic, or hydrogen bonds to a label such that the presence of the probe may be detected by detecting the presence of the label bound to the probe. Alternatively, a method using high affinity interactions may achieve the same results where one of a pair of binding partners binds to the other, e.g., biotin, streptavidin.

The terms “identical” or percent sequence “identity,” in the context of two or more nucleic acids or polypeptide sequences, refer to two or more sequences or subsequences that are the same or have a specified percentage of amino acid residues or nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection (see, e.g., NCBI web site at ncbi.nlm nih.gov/BLAST/ or the like). Such sequences are then said to be “substantially identical.” This definition also refers to, or may be applied to, the compliment of a test sequence. The definition also includes sequences that have deletions and/or additions, as well as those that have substitutions. Employed algorithms can account for gaps and the like.

For sequence comparisons, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Preferably, default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well known in the art. Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Current Protocols in Molecular Biology (Ausubel et al., eds. 1995 supplement)).

A preferred example of algorithm that is suitable for determining percent sequence identity and sequence similarity are the BLAST and BLAST 2.0 algorithms, which are described in Altschul et al., Nuc. Acids Res. 25:3389-3402 (1977) and Altschul et al., J. Mol. Biol. 215:403-410 (1990), respectively.

The phrase “selectively (or specifically) hybridizes to” refers to the binding, duplexing, or hybridizing of a molecule only to a particular nucleotide sequence with a higher affinity, e.g., under more stringent conditions, than to other nucleotide sequences (e.g., total cellular or library DNA or RNA).

The phrase “stringent hybridization conditions” refers to conditions under which a nucleic acid will hybridize to its target sequence, typically in a complex mixture of nucleic acids, but to no other sequences. Stringent conditions are sequence-dependent and will be different in different circumstances. Longer sequences hybridize specifically at higher temperatures. An extensive guide to the hybridization of nucleic acids is found in Tijssen, Techniques in Biochemistry and Molecular Biology—Hybridization with Nucleic Probes, “Overview of principles of hybridization and the strategy of nucleic acid assays” (1993). Generally, stringent hybridization conditions are selected to be about 5-10° C. lower than the thermal melting point (Tm) for the specific sequence at a defined ionic strength pH. The Tm is the temperature (under defined ionic strength, pH, and nucleic concentration) at which 50% of the probes complementary to the target hybridize to the target sequence at equilibrium (as the target sequences are present in excess, at Tm, 50% of the probes are occupied at equilibrium). Stringent hybridization conditions may also be achieved with the addition of destabilizing agents such as formamide. For selective or specific hybridization, a positive signal is at least two times background, preferably 10 times background hybridization. Exemplary stringent hybridization conditions can be as following: 50% formamide, 5×SSC, and 1% SDS, incubating at 42° C., or, 5×SSC, 1% SDS, incubating at 65° C., with wash in 0.2×SSC, and 0.1% SDS at 65° C. Exemplary “moderately stringent hybridization conditions” include a hybridization in a buffer of 40% formamide, 1 M NaCl, 1% SDS at 37° C., and a wash in 1×SSC at 45° C. A positive hybridization is at least twice background. Those of ordinary skill will readily recognize that alternative hybridization and wash conditions can be utilized to provide conditions of similar stringency. Additional guidelines for determining hybridization parameters are provided in numerous reference, e.g., and Current Protocols in Molecular Biology, ed. Ausubel, et al., John Wiley & Sons.

Nucleic acids may be substantially identical if the polypeptides which they encode are substantially identical. This occurs, for example, when a copy of a nucleic acid is created using the maximum codon degeneracy permitted by the genetic code. In such cases, the nucleic acids typically hybridize under moderately stringent hybridization conditions.

An “inhibitory nucleic acid” is a nucleic acid (e.g. DNA, RNA, polymer of nucleotide analogs) that is capable of binding to a target nucleic acid (e.g. an mRNA translatable into PTPRS) and reducing transcription of the target nucleic acid (e.g. mRNA from DNA) or reducing the translation of the target nucleic acid (e.g. mRNA) or altering transcript splicing (e.g. single stranded morpholino oligo). A “morpholino oligo” may be alternatively referred to as a “morpholino nucleic acid” and refers to morpholine-containing nucleic acid nucleic acids commonly known in the art (e.g. phosphoramidate morpholino oligo or a “PMO”). See Marcos, P., Biochemical and Biophysical Research Communications 358 (2007) 521-527. In some embodiments, the “inhibitory nucleic acid” is a nucleic acid that is capable of binding (e.g. hybridizing) to a target nucleic acid (e.g. an mRNA translatable into an RPTPsigma) and reducing translation of the target nucleic acid. The target nucleic acid is or includes one or more target nucleic acid sequences to which the inhibitory nucleic acid binds (e.g. hybridizes). Thus, an inhibitory nucleic acid typically is or includes a sequence (also referred to herein as an “antisense nucleic acid sequence”) that is capable of hybridizing to at least a portion of a target nucleic acid at a target nucleic acid sequence, An example of an inhibitory nucleic acid is an antisense nucleic acid. Another example of an inhibitory nucleic acid is siRNA or RNAi (including their derivatives or pre-cursors, such as nucleotide analogs). Further examples include shRNA, miRNA, shmiRNA, or certain of their derivatives or pre-cursors. In some embodiments, the inhibitory nucleic acid is single stranded. In other embodiments, the inhibitory nucleic acid is double stranded.

An “antisense nucleic acid” is a nucleic acid (e.g. DNA, RNA or analogs thereof) that is at least partially complementary to at least a portion of a specific target nucleic acid (e.g. a target nucleic acid sequence), such as an mRNA molecule (e.g. a target mRNA molecule) (see, e.g., Weintraub, Scientific American, 262:40 (1990)), for example antisense, siRNA, shRNA, shmiRNA, miRNA (microRNA). Thus, antisense nucleic acids are capable of hybridizing to (e.g. selectively hybridizing to) a target nucleic acid (e.g. target mRNA). In some embodiments, the antisense nucleic acid hybridizes to the target nucleic acid sequence (e.g. mRNA) under stringent hybridization conditions. In some embodiments, the antisense nucleic acid hybridizes to the target nucleic acid (e.g. mRNA) under moderately stringent hybridization conditions. Antisense nucleic acids may comprise naturally occurring nucleotides or modified nucleotides such as, e.g., phosphorothioate, methylphosphonate, and -anomeric sugar-phosphate, backbone-modified nucleotides. An “anti-PTPRS antisense nucleic acid” is an antisense nucleic acid that is at least partially complementary to at least a portion of a target nucleic acid sequence, such as an mRNA molecule, that codes at least a portion of the PTPRS. In some embodiments, an antisense nucleic acid is a morpholino oligo. In some embodiments, a morpholino oligo is a single stranded antisense nucleic acid, as is known in the art. In some embodiments, a morpholino oligo decreases protein expression of a target, reduces translation of the target mRNA, reduces translation initiation of the target mRNA, or modifies transcript splicing. In some embodiments, the morpholino oligo is conjugated to a cell permeable moiety (e.g. peptide). Antisense nucleic acids may be single or double stranded nucleic acids.

In the cell, the antisense nucleic acids may hybridize to the target mRNA, forming a double-stranded molecule. The antisense nucleic acids, interfere with the translation of the mRNA, since the cell will not translate an mRNA that is double-stranded. The use of antisense methods to inhibit the in vitro translation of genes is well known in the art (Marcus-Sakura, Anal. Biochem., 172:289, (1988)). Antisense molecules which bind directly to the DNA may be used.

Inhibitory nucleic acids can be delivered to the subject using any appropriate means known in the art, including by injection, inhalation, or oral ingestion. Another suitable delivery system is a colloidal dispersion system such as, for example, macromolecule complexes, nanocapsules, microspheres, beads, and lipid-based systems including oil-in-water emulsions, micelles, mixed micelles, and liposomes. An example of a colloidal system of this invention is a liposome. Liposomes are artificial membrane vesicles which are useful as delivery vehicles in vitro and in vivo. Nucleic acids, including RNA and DNA within liposomes and be delivered to cells in a biologically active form (Fraley, et al., Trends Biochem. Sci., 6:77, 1981). Liposomes can be targeted to specific cell types or tissues using any means known in the art. Inhibitory nucleic acids (e.g. antisense nucleic acids, morpholino oligos) may be delivered to a cell using cell permeable delivery systems (e.g. cell permeable peptides). In some embodiments, inhibitory nucleic acids are delivered to specific cells or tissues using viral vectors or viruses.

An “siRNA” refers to a nucleic acid that forms a double stranded RNA, which double stranded RNA has the ability to reduce or inhibit expression of a gene or target gene when the siRNA is present (e.g. expressed) in the same cell as the gene or target gene. The siRNA is typically about 5 to about 100 nucleotides in length, more typically about 10 to about 50 nucleotides in length, more typically about 15 to about 30 nucleotides in length, most typically about 20-30 base nucleotides, or about 20-25 or about 24-29 nucleotides in length, e.g., 20, 21, 22, 23, 24, 25, 26, 27, 28, 29, or 30 nucleotides in length. siRNA molecules and methods of generating them are described in, e.g., Bass, 2001, Nature, 411, 428-429; Elbashir et al., 2001, Nature, 411, 494-498; WO 00/44895; WO 01/36646; WO 99/32619; WO 00/01846; WO 01/29058; WO 99/07409; and WO 00/44914. A DNA molecule that transcribes dsRNA or siRNA (for instance, as a hairpin duplex) also provides RNAi. DNA molecules for transcribing dsRNA are disclosed in U.S. Pat. No. 6,573,099, and in U.S. Patent Application Publication Nos. 2002/0160393 and 2003/0027783, and Tuschl and Borkhardt, Molecular Interventions, 2:158 (2002).

The siRNA can be administered directly or siRNA expression vectors can be used to induce RNAi that have different design criteria. A vector can have inserted two inverted repeats separated by a short spacer sequence and ending with a string of T's which serve to terminate transcription.

Construction of suitable vectors containing the nucleic acid sequences employs standard ligation and restriction techniques, which are well understood in the art (see Maniatis et al., in Molecular Cloning: A Laboratory Manual, Cold Spring Harbor Laboratory, New York (1982)). Isolated plasmids, DNA sequences, or synthesized oligonucleotides are cleaved, tailored, and re-ligated in the form desired.

“Biological sample” or “sample” refer to materials obtained from or derived from a subject or patient. A biological sample includes sections of tissues such as biopsy and autopsy samples, and frozen sections taken for histological purposes. Such samples include bodily fluids such as blood and blood fractions or products (e.g., serum, plasma, platelets, red blood cells, and the like), sputum, tissue, cultured cells (e.g., primary cultures, explants, and transformed cells) stool, urine, synovial fluid, joint tissue, synovial tissue, synoviocytes, fibroblast-like synoviocytes, macrophage-like synoviocytes, immune cells, hematopoietic cells, fibroblasts, macrophages, T cells, etc. A biological sample is typically obtained from a eukaryotic organism, such as a mammal such as a primate e.g., chimpanzee or human; cow; dog; cat; a rodent, e.g., guinea pig, rat, mouse; rabbit; or a bird; reptile; or fish.

A “biopsy” refers to the process of removing a tissue sample for diagnostic or prognostic evaluation, and to the tissue specimen itself. Any biopsy technique known in the art can be applied to the diagnostic and prognostic methods of the present invention. The biopsy technique applied will depend on the tissue type to be evaluated (i.e., prostate, lymph node, liver, bone marrow, blood cell, joint tissue, synovial tissue, synoviocytes, fibroblast-like synoviocytes, macrophage-like synoviocytes, immune cells, hematopoietic cells, fibroblasts, macrophages, T cells, etc.), among other factors. Representative biopsy techniques include excisional biopsy, incisional biopsy, needle biopsy, surgical biopsy, and bone marrow biopsy. Biopsy techniques are discussed, for example, in Harrison's Principles of Internal Medicine, Kasper, et al., eds., 16th ed., 2005, Chapter 70, and throughout Part V.

The terms “polypeptide,” “peptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, carboxyglutamate, and O-phosphoserine Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

“Conservatively modified variants” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence with respect to the expression product, but not with respect to actual probe sequences.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention.

The following eight groups each contain amino acids that are conservative substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and 8) Cysteine (C), Methionine (M) (see, e.g., Creighton, Proteins (1984)).

A “label” or a “detectable moiety” is a composition detectable by spectroscopic, photochemical, biochemical, immunochemical, chemical, or other physical means. For example, useful labels include 32P, fluorescent dyes, electron-dense reagents, enzymes (e.g., as commonly used in an ELISA), biotin, digoxigenin, or haptens and proteins or other entities which can be made detectable, e.g., by incorporating a radiolabel into a peptide or antibody specifically reactive with a target peptide. Any method known in the art for conjugating an antibody to the label may be employed, e.g., using methods described in Hermanson, Bioconjugate Techniques 1996, Academic Press, Inc., San Diego.

The term “recombinant” when used with reference, e.g., to a cell, or nucleic acid, protein, or vector, indicates that the cell, nucleic acid, protein or vector has been modified by or is the result of laboratory methods. Thus, for example, recombinant proteins include proteins produced by laboratory methods. Recombinant proteins can include amino acid residues not found within the native (non-recombinant) form of the protein or can be include amino acid residues that have been modified, e.g., labeled.

The term “heterologous” when used with reference to portions of a nucleic acid indicates that the nucleic acid comprises two or more subsequences that are not found in the same relationship to each other in nature. For instance, the nucleic acid is typically recombinantly produced, having two or more sequences from unrelated genes arranged to make a new functional nucleic acid, e.g., a promoter from one source and a coding region from another source. Similarly, a heterologous protein indicates that the protein comprises two or more subsequences that are not found in the same relationship to each other in nature (e.g., a fusion protein).

The polypeptide can be chemically linked to another molecule. As used herein, the terms “bioconjugate” and “bioconjugate linker” refers to the resulting association between atoms or molecules of “bioconjugate reactive groups” or “bioconjugate reactive moieties”. The association can be direct or indirect. For example, a conjugate between a first bioconjugate reactive group (e.g., —NH2, —C(O)OH, —N-hydroxysuccinimide, or -maleimide) and a second bioconjugate reactive group (e.g., sulfhydryl, sulfur-containing amino acid, amine, amine sidechain containing amino acid, or carboxylate) provided herein can be direct, e.g., by covalent bond or linker (e.g. a first linker of second linker), or indirect, e.g., by non-covalent bond (e.g. electrostatic interactions (e.g. ionic bond, hydrogen bond, halogen bond), van der Waals interactions (e.g. dipole-dipole, dipole-induced dipole, London dispersion), ring stacking (pi effects), hydrophobic interactions and the like). In embodiments, bioconjugates or bioconjugate linkers are formed using bioconjugate chemistry (i.e. the association of two bioconjugate reactive groups) including, but are not limited to nucleophilic substitutions (e.g., reactions of amines and alcohols with acyl halides, active esters), electrophilic substitutions (e.g., enamine reactions) and additions to carbon-carbon and carbon-heteroatom multiple bonds (e.g., Michael reaction, Diels-Alder addition). These and other useful reactions are discussed in, for example, March, ADVANCED ORGANIC CHEMISTRY, 3rd Ed., John Wiley & Sons, New York, 1985; Hermanson, BIOCONJUGATE TECHNIQUES, Academic Press, San Diego, 1996; and Feeney et al., MODIFICATION OF PROTEINS; Advances in Chemistry Series, Vol. 198, American Chemical Society, Washington, D.C., 1982. In embodiments, the first bioconjugate reactive group (e.g., maleimide moiety) is covalently attached to the second bioconjugate reactive group (e.g. a sulfhydryl). In embodiments, the first bioconjugate reactive group (e.g., haloacetyl moiety) is covalently attached to the second bioconjugate reactive group (e.g. a sulfhydryl). In embodiments, the first bioconjugate reactive group (e.g., pyridyl moiety) is covalently attached to the second bioconjugate reactive group (e.g. a sulfhydryl). In embodiments, the first bioconjugate reactive group (e.g., —N-hydroxysuccinimide moiety) is covalently attached to the second bioconjugate reactive group (e.g. an amine). In embodiments, the first bioconjugate reactive group (e.g., maleimide moiety) is covalently attached to the second bioconjugate reactive group (e.g. a sulfhydryl). In embodiments, the first bioconjugate reactive group (e.g., -sulfo-N-hydroxysuccinimide moiety) is covalently attached to the second bioconjugate reactive group (e.g. an amine).

The terms “bind” and “bound” as used herein is used in accordance with its plain and ordinary meaning and refers to the association between atoms or molecules. The association can be direct or indirect. For example, bound atoms or molecules may be direct, e.g., by covalent bond or linker (e.g. a first linker or second linker), or indirect, e.g., by non-covalent bond (e.g. electrostatic interactions (e.g. ionic bond, hydrogen bond, halogen bond), van der Waals interactions (e.g. dipole-dipole, dipole-induced dipole, London dispersion), ring stacking (pi effects), hydrophobic interactions and the like).

“Antibody” refers to a polypeptide comprising a framework region from an immunoglobulin gene or fragments thereof that specifically binds and recognizes an antigen. The recognized immunoglobulin genes include the kappa, lambda, alpha, gamma, delta, epsilon, and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively. Typically, the antigen-binding region of an antibody will be most critical in specificity and affinity of binding. In some embodiments, antibodies or fragments of antibodies may be derived from different organisms, including humans, mice, rats, hamsters, camels, etc. Antibodies of the invention may include antibodies that have been modified or mutated at one or more amino acid positions to improve or modulate a desired function of the antibody (e.g. glycosylation, expression, antigen recognition, effector functions, antigen binding, specificity, etc.).

An exemplary immunoglobulin (antibody) structural unit comprises a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (VL) and variable heavy chain (VH) refer to these light and heavy chains respectively.

Antibodies exist, e.g., as intact immunoglobulins or as a number of well-characterized fragments produced by digestion with various peptidases. Thus, for example, pepsin digests an antibody below the disulfide linkages in the hinge region to produce F(ab)′2, a dimer of Fab which itself is a light chain joined to VH-CH1 by a disulfide bond. The F(ab)′2 may be reduced under mild conditions to break the disulfide linkage in the hinge region, thereby converting the F(ab)′2 dimer into an Fab′ monomer. The Fab′ monomer is essentially Fab with part of the hinge region (see Fundamental Immunology (Paul ed., 3d ed. 1993). While various antibody fragments are defined in terms of the digestion of an intact antibody, one of skill will appreciate that such fragments may be synthesized de novo either chemically or by using recombinant DNA methodology. Thus, the term antibody, as used herein, also includes antibody fragments either produced by the modification of whole antibodies, or those synthesized de novo using recombinant DNA methodologies (e.g., single chain Fv) or those identified using phage display libraries (see, e.g., McCafferty et al., Nature 348:552-554 (1990)).

For preparation of suitable antibodies of the invention and for use according to the invention, e.g., recombinant, monoclonal, or polyclonal antibodies, many techniques known in the art can be used (see, e.g., Kohler & Milstein, Nature 256:495-497 (1975); Kozbor et al., Immunology Today 4: 72 (1983); Cole et al., pp. 77-96 in Monoclonal Antibodies and Cancer Therapy, Alan R. Liss, Inc. (1985); Coligan, Current Protocols in Immunology (1991); Harlow & Lane, Antibodies, A Laboratory Manual (1988); and Goding, Monoclonal Antibodies: Principles and Practice (2d ed. 1986)). The genes encoding the heavy and light chains of an antibody of interest can be cloned from a cell, e.g., the genes encoding a monoclonal antibody can be cloned from a hybridoma and used to produce a recombinant monoclonal antibody. Gene libraries encoding heavy and light chains of monoclonal antibodies can also be made from hybridoma or plasma cells. Random combinations of the heavy and light chain gene products generate a large pool of antibodies with different antigenic specificity (see, e.g., Kuby, Immunology (3rd ed. 1997)). Techniques for the production of single chain antibodies or recombinant antibodies (U.S. Pat. Nos. 4,946,778, 4,816,567) can be adapted to produce antibodies to polypeptides of this invention. Also, transgenic mice, or other organisms such as other mammals, may be used to express humanized or human antibodies (see, e.g., U.S. Pat. Nos. 5,545,807; 5,545,806; 5,569,825; 5,625,126; 5,633,425; 5,661,016, Marks et al., Bio/Technology 10:779-783 (1992); Lonberg et al., Nature 368:856-859 (1994); Morrison, Nature 368:812-13 (1994); Fishwild et al., Nature Biotechnology 14:845-51 (1996); Neuberger, Nature Biotechnology 14:826 (1996); and Lonberg & Huszar, Intern. Rev. Immunol. 13:65-93 (1995)). Alternatively, phage display technology can be used to identify antibodies and heteromeric Fab fragments that specifically bind to selected antigens (see, e.g., McCafferty et al., Nature 348:552-554 (1990); Marks et al., Biotechnology 10:779-783 (1992)). Antibodies can also be made bispecific, i.e., able to recognize two different antigens (see, e.g., WO 93/08829, Traunecker et al., EMBO J. 10:3655-3659 (1991); and Suresh et al., Methods in Enzymology 121:210 (1986)). Antibodies can also be heteroconjugates, e.g., two covalently joined antibodies, or immunotoxins (see, e.g., U.S. Pat. No. 4,676,980, WO 91/00360; WO 92/200373; and EP 03089).

Methods for humanizing or primatizing non-human antibodies are well known in the art (e.g., U.S. Pat. Nos. 4,816,567; 5,530,101; 5,859,205; 5,585,089; 5,693,761; 5,693,762; 5,777,085; 6,180,370; 6,210,671; and 6,329,511; WO 87/02671; EP Patent Application 0173494; Jones et al. (1986) Nature 321:522; and Verhoyen et al. (1988) Science 239:1534). Humanized antibodies are further described in, e.g., Winter and Milstein (1991) Nature 349:293. Generally, a humanized antibody has one or more amino acid residues introduced into it from a source which is non-human. These non-human amino acid residues are often referred to as import residues, which are typically taken from an import variable domain. Humanization can be essentially performed following the method of Winter and co-workers (see, e.g., Morrison et al., PNAS USA, 81:6851-6855 (1984), Jones et al., Nature 321:522-525 (1986); Riechmann et al., Nature 332:323-327 (1988); Morrison and Oi, Adv. Immunol., 44:65-92 (1988), Verhoeyen et al., Science 239:1534-1536 (1988) and Presta, Curr. Op. Struct. Biol. 2:593-596 (1992), Padlan, Molec. Immun., 28:489-498 (1991); Padlan, Molec. Immun., 31(3):169-217 (1994)), by substituting rodent CDRs or CDR sequences for the corresponding sequences of a human antibody. Accordingly, such humanized antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies. For example, polynucleotides comprising a first sequence coding for humanized immunoglobulin framework regions and a second sequence set coding for the desired immunoglobulin complementarity determining regions can be produced synthetically or by combining appropriate cDNA and genomic DNA segments. Human constant region DNA sequences can be isolated in accordance with well known procedures from a variety of human cells.

A “chimeric antibody” is an antibody molecule in which (a) the constant region, or a portion thereof, is altered, replaced or exchanged so that the antigen binding site (variable region) is linked to a constant region of a different or altered class, effector function and/or species, or an entirely different molecule which confers new properties to the chimeric antibody, e.g., an enzyme, toxin, hormone, growth factor, drug, etc.; or (b) the variable region, or a portion thereof, is altered, replaced or exchanged with a variable region having a different or altered antigen specificity. The preferred antibodies of, and for use according to the invention include humanized and/or chimeric monoclonal antibodies.

Techniques for conjugating therapeutic agents to antibodies are well known (see, e.g., Amon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. 1985); Hellstrom et al., “Antibodies For Drug Delivery” in Controlled Drug Delivery (2^{nd} Ed.), Robinson et al. (eds.), pp. 623-53 (Marcel Dekker, Inc. 1987); Thorpe, “Antibody Carriers Of Cytotoxic Agents In Cancer Therapy: A Review” in Monoclonal Antibodies '84: Biological And Clinical Applications, Pinchera et al. (eds.), pp. 475-506 (1985); and Thorpe et al., “The Preparation And Cytotoxic Properties Of Antibody-Toxin Conjugates”, Immunol. Rev., 62:119-58 (1982)).

The phrase “specifically (or selectively) binds” to an antibody or “specifically (or selectively) immunoreactive with,” when referring to a protein or peptide, refers to a binding reaction that is determinative of the presence of the protein, often in a heterogeneous population of proteins and other biologics. Thus, under designated immunoassay conditions, the specified antibodies bind to a particular protein at least two times the background and more typically more than 10 to 100 times background. Specific binding to an antibody under such conditions requires an antibody that is selected for its specificity for a particular protein. For example, polyclonal antibodies can be selected to obtain only those polyclonal antibodies that are specifically immunoreactive with the selected antigen and not with other proteins. This selection may be achieved by subtracting out antibodies that cross-react with other molecules. A variety of immunoassay formats may be used to select antibodies specifically immunoreactive with a particular protein. For example, solid-phase ELISA immunoassays are routinely used to select antibodies specifically immunoreactive with a protein (see, e.g., Harlow & Lane, Using Antibodies, A Laboratory Manual (1998) for a description of immunoassay formats and conditions that can be used to determine specific immunoreactivity).

As used herein, the term “pharmaceutically acceptable” is used synonymously with “physiologically acceptable” and “pharmacologically acceptable”. A pharmaceutical composition will generally comprise agents for buffering and preservation in storage, and can include buffers and carriers for appropriate delivery, depending on the route of administration.

Certain compounds of the present invention can exist in unsolvated forms as well as solvated forms, including hydrated forms. In general, the solvated forms are equivalent to unsolvated forms and are intended to be encompassed within the scope of the present invention. Certain compounds of the present invention may exist in multiple crystalline or amorphous forms. In general, all physical forms are equivalent for the uses contemplated by the present invention and are intended to be within the scope of the present invention.

“PTPR” or “RPTP” or “rPTP” (all terms are equal) refer to receptor protein tyrosine phosphatases, which are found in nature as protein tyrosine phosphatases.

“PTPRS” refers to protein tyrosine phosphatase receptor type S (or sigma), which is a member of the protein tyrosine phosphatase (PTP) family. The amino acid sequence of PTPRS can be found, for example, at UniProtKB/Swiss-Prot Accession No. Q13332 and BOV2N1, and also SEQ ID NO:4. The nucleic acid sequence of PTPRS can be found, for example, at GenBank Accession No. NC_000019.9 and. PTPRS includes an intracellular domain, e.g., amino acid residues 1304-1948 of SEQ ID NO:8 or amino acid residues 1279-1907 of SEQ ID NO:4, a transmembrane domain, e.g., amino acid residues 1283-1303 of SEQ ID NO:8 or amino acid residues 1258-1278 of SEQ ID NO:4, and an extracellular domain, e.g., SEQ ID NO:9 or SEQ ID NO:10. The term transmembrane domain refers to the portion of a protein or polypeptide that is embedded in and, optionally, spans a membrane. The term intracellular domain refers to the portion of a protein or polypeptide that extends into the cytoplasm of a cell. The term extracellular domain refers to the portion of a protein or polypeptide that extends into the extracellular environment. The extracellular domain of PTPRS includes immunoglobulin-like domain 1 (Ig1), immunoglobulin-like domain 2 (Ig2) and immunoglobulin-like domain 2 (Ig3). The amino acid sequence of Ig1 includes amino acid residues 30 to 127 of SEQ ID NO:4 or amino acid residues 30-127 of SEQ ID NO:8, or the amino acid sequence EEPRFIKEPKDQIGVSGGVASFVCQATGDPKPRVTWNKKGKKVNSQRFETIEFDESA GAVLRIQPLRTPRDENVYECVAQNSVGEITVHAKLTVLRE (SEQ ID NO:1) or the amino acid sequence of SEQ ID NO:5. The amino acid sequence of Ig2 includes amino acid residues 128 to 231 of SEQ ID NO:4, or amino acid residues 128-244 of SEQ ID NO:8, or the amino acid sequence DQLPSGFPNIDMGPQLKVVERTRTATMLCAASGNPDPEITWFKDFLPVDPSASNGRI KQLRSETFESTPIRGALQIESSEETDQGKYECVATNSAGVRYSSPANLYVRVRRVA (SEQ ID NO:2) or the amino acid sequence of SEQ ID NO:6. The amino acid sequence of Ig3 includes amino acid residues 232 to 321 of SEQ ID NO:4, or amino acid residues 245-334 of SEQ ID NO:8 or the amino acid sequence PRFSILPMSHEIMPGGNVNITCVAVGSPMPYVKWMQGAEDLTPEDDMPVGRNVLEL TDVKDSANYTCVAMSSLGVIEAVAQITVKSLPKA (SEQ ID NO:3) or the amino acid sequence of SEQ ID NO:7. [NEED DESCRIPTION OF Ig1&2 and Ig1&2-Fc CONSTRUCTS AND FULL SEQUENCES OF BOTH]

A “protein level of an RPTP” refers to an amount (relative or absolute) of RPTP in its protein form (as distinguished from its precursor RNA form). A protein of an RPTP may include a full-length protein (e.g. the protein translated from the complete coding region of the gene, which may also include post-translational modifications), functional fragments of the full length protein (e.g. sub-domains of the full length protein that possess an activity or function in an assay), or protein fragments of the RPTP, which may be any peptide or oligopeptide of the full length protein.

An “RNA level of an RPTP” refers to an amount (relative or absolute) of RNA present that may be translated to form an RPTP. The RNA of an RPTP may be a full-length RNA sufficient to form a full-length RPTP. The RNA of an RPTP may also be a fragment of the full length RNA thereby forming a fragment of the full length RPTP. The fragment of the full length RNA may form a functional fragment of the RPTP. In some embodiments, the RNA of an RPTP includes all splice variants of an RPTP gene. Splice variants of PTPRS are provided in Pulido R, Serra-Pages C et al., “The LAR/PTP delta/PTP sigma subfamily of transmembrane protein-tyrosine-phosphatases: multiple human LAR, PTP delta, and PTP sigma isoforms are expressed in a tissue-specific manner and associate with the LAR-interacting protein LIP.1.” Proc Natl Acad Sci USA. 1995, Dec. 5; 92(25):11686-90.

An “autoimmune therapeutic agent” is a molecule (e.g. antibody, nucleic acid, inhibitory nucleic acid, synthetic chemical, small chemical molecule) that treats or prevents an autoimmune disease when administered to a subject in a therapeutically effective dose or amount. In some embodiments, an autoimmune therapeutic agent is an RPTP binding agent.

Tumor necrosis factor (TNF, cachexia, cachectin, tumor necrosis factor alpha, TNF-alpha, or TNFα) is a cell signaling protein (cytokine) involved in systemic inflammation and is one of the cytokines that make up the acute phase reaction. It is produced chiefly by activated macrophages, although it can be produced by many other cell types such as CD4+ lymphocytes, NK cells, neutrophils, mast cells, eosinophils, and neurons. TNF is a member of the TNF superfamily, consisting of various transmembrane proteins with a homologous TNF domain.

Interleukins (ILs) are a group of cytokines (secreted proteins and signal molecules) that were first seen to be expressed by white blood cells (leukocytes). ILs can be divided into four major groups based on distinguishing structural features. However, their amino acid sequence similarity is rather weak (typically 15-25% identity). The human genome encodes more than 50 interleukins and related proteins. The function of the immune system depends in a large part on interleukins, and rare deficiencies of a number of them have been described, all featuring autoimmune diseases or immune deficiency. The majority of interleukins are synthesized by helper CD4 T lymphocytes, as well as through monocytes, macrophages, and endothelial cells. They promote the development and differentiation of T and B lymphocytes, and hematopoietic cells.

Interleukin 6 (IL-6) is an interleukin that acts as both a pro-inflammatory cytokine and an anti-inflammatory myokine. In humans, it is encoded by the IL-6 gene. In addition, osteoblasts secrete IL-6 to stimulate osteoclast formation. Smooth muscle cells in the tunica media of many blood vessels also produce IL-6 as a pro-inflammatory cytokine. The role of IL-6 as an anti-inflammatory myokine is mediated through its inhibitory effects on TNF-alpha and IL-1, and activation of IL-1ra and IL-10.

As defined herein, the term “inhibition”, “inhibit”, “inhibiting” and the like in reference to a protein-inhibitor interaction means negatively affecting (e.g. decreasing) the activity or function of the protein relative to the activity or function of the protein in the absence of the inhibitor. In embodiments inhibition means negatively affecting (e.g. decreasing) the concentration or levels of the protein relative to the concentration or level of the protein in the absence of the inhibitor. In embodiments inhibition refers to reduction of a disease or symptoms of disease. In embodiments, inhibition refers to a reduction in the activity of a particular protein target. Thus, inhibition includes, at least in part, partially or totally blocking stimulation, decreasing, preventing, or delaying activation, or inactivating, desensitizing, or down-regulating signal transduction or enzymatic activity or the amount of a protein. In embodiments, inhibition refers to a reduction of activity of a target protein resulting from a direct interaction (e.g. an inhibitor binds to the target protein). In embodiments, inhibition refers to a reduction of activity of a target protein from an indirect interaction (e.g. an inhibitor binds to a protein that activates the target protein, thereby preventing target protein activation). While not wishing to be held by theory, in some embodiments, the inhibitor acts by removing protein from or bringing protein to its correct site of action and/or in the vicinity of its substrate

The terms “inhibitor,” “repressor” or “antagonist” or “downregulator” interchangeably refer to a substance capable of detectably decreasing the expression or activity of a given gene or protein. The antagonist can decrease expression or activity 10%, 20%, 30%, 40%, 50%, 60%, 70%, 80%, 90% or more in comparison to a control in the absence of the antagonist. In certain instances, expression or activity is 1.5-fold, 2-fold, 3-fold, 4-fold, 5-fold, 10-fold or lower than the expression or activity in the absence of the antagonist.

Interleukin 6 (IL-6) is an interleukin that acts as both a pro-inflammatory cytokine and an anti-inflammatory myokine. IL-6 signals through a cell-surface type I cytokine receptor complex consisting of the ligand-binding IL-6Ra chain (CD126), and the signal-transducing component gp130 (also called CD130). CD130 is the common signal transducer for several cytokines including leukemia inhibitory factor (LIF), ciliary neurotropic factor, oncostatin M, IL-11 and cardiotrophin-1, and is almost ubiquitously expressed in most tissues. IL-6 inhibitors can be directed to IL-6 or its receptor.

Tocilizumab, or atlizumab, is an immunosuppressive drug, mainly for the treatment of rheumatoid arthritis (RA) and systemic juvenile idiopathic arthritis, a severe form of arthritis in children. Tocilizumab is a humanized monoclonal antibody against the interleukin-6 receptor (IL-6R). This drug is prescribed for adult patients with moderately to severely active rheumatoid arthritis (RA) who have had an inadequate response to one or more disease-modifying anti-rheumatic drugs (DMARDs). Effective doses: For treatment of RA by IV, the dosage in adults is 4 mg/kg IV as a 60-minute single drip infusion once every 4 weeks, followed by an increase to 8 mg/kg IV given once every 4 weeks as a 60-minute single drip infusion based on clinical response. Reduction from 8 mg/kg to 4 mg/kg is recommended for management of certain dose-related laboratory changes including elevated liver enzymes, neutropenia, and thrombocytopenia. The maximum recommended dose is 800 mg per infusion. For treatment of RA by SC, in patients less than 100 kg, 162 mg subcutaneously every other week, followed by an increase to every week based on clinical response. For patients 100 kg or greater, 162 mg subcutaneously every week. Interruption of dose or reduction in frequency of administration from every week to every other week is recommended for certain dose-related laboratory changes (e.g., elevated liver enzymes, neutropenia, thrombocytopenia).

Sarilumab, or Kevzara is a human monoclonal antibody against the interleukin-6 receptor, to reduce symptoms and slow the progression of structural damage in moderately to severely active RA, in patients 18 years of age or older who have failed one or more disease modifying antirheumatic drugs (DMARDs). Effective doses: Dosage for rheumatoid arthritis in adults is 100 mg subcutaneously once daily.

Tumor necrosis factor (TNF, cachexia, or cachectin; tumor necrosis factor alpha or TNFα) is a cell signaling protein (cytokine) involved in systemic inflammation and is one of the cytokines that make up the acute phase reaction. It is produced chiefly by activated macrophages, although it can be produced by many other cell types such as CD4+ lymphocytes, NK cells, neutrophils, mast cells, eosinophils, and neurons. TNF is a member of the TNF superfamily, consisting of various transmembrane proteins with a homologous TNF domain. TNF can bind two receptors, TNFR1 (TNF receptor type 1; CD120a; p55/60) and TNFR2 (TNF receptor type 2; CD120b; p75/80). TNFR1 is 55-kDa and TNFR2 is 75-kDa. TNFR1 is expressed in most tissues, and can be fully activated by both the membrane-bound and soluble trimeric forms of TNF, whereas TNFR2 is found typically in cells of the immune system, and respond to the membrane-bound form of the TNF homotrimer. TNF promotes the inflammatory response, which, in turn, causes many of the clinical problems associated with autoimmune disorders such as rheumatoid arthritis, ankylosing spondylitis, inflammatory bowel disease, psoriasis, hidradenitis suppurativa and refractory asthma.

TNF inhibitors can be used to treat the above conditions. This inhibition can be achieved with a monoclonal antibody such as infliximab (Remicade) binding directly to TNFα, adalimumab (Humira), certolizumab pegol (Cimzia) or with a decoy circulating receptor fusion protein such as etanercept (Enbrel) which binds to TNFα with greater affinity than the TNFR.

Etanercept (or Enbrel), or its biosimilar (e.g. Benepali), is a biopharmaceutical that treats autoimmune diseases by interfering with tumor necrosis factor (TNF) by acting as a TNF inhibitor. Etanercept is used to treat rheumatoid arthritis, juvenile idiopathic arthritis and psoriatic arthritis, plaque psoriasis and ankylosing spondylitis, by inhibiting TNF-alpha. Etanercept is a fusion protein produced by recombinant DNA. It fuses the TNF receptor to the constant end of the IgG1 antibody. It is a large molecule, with a molecular weight of 150 kDa., that binds to TNFα and decreases its role in disorders involving excess inflammation in humans and other animals, including autoimmune diseases such as ankylosing spondylitis, juvenile rheumatoid arthritis, psoriasis, psoriatic arthritis, rheumatoid arthritis, and, potentially, in a variety of other disorders mediated by excess TNFα. Effective dosage for rheumatoid arthritis in adults: 50 mg subcutaneously once a week or 25 mg subcutaneously twice a week.

Adalimumab, or Humira, or its biosimilar, is a monoclonal antibody against TNFα used to treat rheumatoid arthritis, psoriatic arthritis, ankylosing spondylitis, Crohn's disease, ulcerative colitis, psoriasis, hidradenitis suppurativa, uveitis, and juvenile idiopathic arthritis. Adalimumab is a disease-modifying antirheumatic drug and monoclonal antibody that works by inactivating tumor necrosis factor-alpha (TNFα). Effective dosage for rheumatoid arthritis in adults: 40 mg subcutaneously every other week; some patients with RA not taking concomitant methotrexate may benefit from increasing the frequency to 40 mg every week.

Infliximab, or Remicade, or its biosimilar, is a monoclonal antibody against TNFα used to treat a number of autoimmune diseases such as Crohn's disease, ulcerative colitis, rheumatoid arthritis, ankylosing spondylitis, psoriasis, psoriatic arthritis, and Behçet's disease. Dosage for rheumatoid arthritis in adults: 3 mg/kg given as an IV induction regimen at 0, 2, and 6 weeks followed by a maintenance regimen of 3 mg/kg IV every 8 weeks thereafter; adjusting the dose up to 10 mg/kg IV or treating as often as every 4 weeks may be considered for patients who have an incomplete response.

Golimumab, or Simponi, or its biosimilar, is a human monoclonal antibody against TNFα which is used as an immunosuppressive drug and marketed under the brand name Simponi. Golimumab is used as a treatment for rheumatoid arthritis, psoriatic arthritis and ankylosing spondylitis. Effective dosage for subcutaneous administration: 50 mg once a month. Effective dosage for IV: 2 mg/kg over 30 minutes at Weeks 0 and 4, then every 8 weeks thereafter.

Certolizumab, Certolizumab pegol, or Cimzia, or its biosimilar, is a monoclonal antibody or a fragment of a monoclonal antibody against TNFα used for the treatment of Crohn's disease, rheumatoid arthritis, psoriatic arthritis and ankylosing spondylitis. Effective dose, initial dose: 400 mg subcutaneously (given as two subcutaneous injections of 200 mg) at weeks 0, 2, and 4, followed by 200 mg subcutaneously every other week. Maintenance dose: 400 mg subcutaneously every 4 weeks in patients who obtain a clinical response. Injection sites should be rotated and injections should not be given in areas where the skin is tender, bruised, red, or hard. When a 400 mg dose is needed (given as 2 subcutaneous injections of 200 mg), injections should occur at separate sites in the thigh or abdomen.

An “RPTP binding agent” is a molecule that binds (e.g. preferentially binds) to RPTP, RNA that is translatable to RPTP, or DNA that is transcribable to an RNA that is translatable to an RPTP. Where the molecule preferentially binds, the binding is preferential as compared to other macromolecular biomolecules present in an organism or cell. A compound preferentially binds to as compared to other macromolecular biomolecules present in an organism or cell, for example, when the preferential binding is 1.1-fold, 1.2-fold, 1.3-fold, 1.4-fold, 1.5-fold, 1.6-fold, 1.7-fold, 1.8-fold, 1.9-fold, 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, 200-fold, 300-fold, 400-fold, 500-fold, 600-fold, 700-fold, 800-fold, 900-fold, 1000-fold, 2000-fold, 3000-fold, 4000-fold, 5000-fold, 6000-fold, 7000-fold, 8000-fold, 9000-fold, 10000-fold, 100,000-fold, 1,000,000-fold greater.

An agent may “target” an RPTP, a nucleic acid (e.g. RNA or DNA) encoding an RPTP, or a protein of an RPTP, by binding (e.g. preferentially binding) to the RPTP, nucleic acid (e.g. RNA or DNA) encoding an RPTP, or protein of an RPTP. Optionally, the RPTP is PTPRS. An agent preferentially binds to a molecule, for example, when the binding to the targeted molecule is greater than the binding to other molecules of a similar form. In some embodiments, the preferential binding is 1.1-fold, 1.2-fold, 1.3-fold, 1.4-fold, 1.5-fold, 1.6-fold, 1.7-fold, 1.8-fold, 1.9-fold, 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, 200-fold, 300-fold, 400-fold, 500-fold, 600-fold, 700-fold, 800-fold, 900-fold, 1000-fold, 2000-fold, 3000-fold, 4000-fold, 5000-fold, 6000-fold, 7000-fold, 8000-fold, 9000-fold, 10000 fold, 100,000-fold, 1,000,000-fold greater. In some embodiments, an agent targets an RPTP, a nucleic acid (e.g. RNA or DNA) of an RPTP, or a protein of an RPTP when a binding assay or experiment (e.g. gel electrophoresis, chromatography, immunoassay, radioactive or non-radioactive labeling, immunoprecipitation, activity assay, etc.) reveals only an interaction or primarily an interaction with a single RPTPS, a nucleic acid (e.g. RNA or DNA) of a single RPTP, or a protein of a single RPTP. An agent may also “target” an RPTP, a nucleic acid (e.g. RNA or DNA) of an RPTP, or a protein of an RPTPS by binding to the RPTP, nucleic acid (e.g. RNA or DNA) of an RPTP, or protein of an RPTP, by decreasing or increasing the amount of RPTP in a cell or organism relative to the absence of the agent, or decreasing the interaction between the RPTP with a physiological or natural ligand. A person having ordinary skill in the art, using the guidance provided herein, may easily determine whether an agent decreases or increases the amount of an RPTP in a cell or organism.

As used herein, “treating” or “treatment of” a condition, disease or disorder or symptoms associated with a condition, disease or disorder refers to an approach for obtaining beneficial or desired results, including clinical results. Beneficial or desired clinical results can include, but are not limited to, alleviation or amelioration of one or more symptoms or conditions, diminishment of extent of condition, disorder or disease, stabilization of the state of condition, disorder or disease, prevention of development of condition, disorder or disease, prevention of spread of condition, disorder or disease, delay or slowing of condition, disorder or disease progression, delay or slowing of condition, disorder or disease onset, amelioration or palliation of the condition, disorder or disease state, and remission, whether partial or total. “Treating” can also mean prolonging survival of a subject beyond that expected in the absence of treatment. “Treating” can also mean inhibiting the progression of the condition, disorder or disease, slowing the progression of the condition, disorder or disease temporarily, although in some instances, it involves halting the progression of the condition, disorder or disease permanently. As used herein the terms treatment, treat, or treating refers to a method of reducing the effects of one or more symptoms of a disease or condition characterized by expression of the protease or symptom of the disease or condition characterized by expression of the protease. Thus in the disclosed method, treatment can refer to a 10%, 20%, 30%, 40%, 50%, 60%, 70%, 80%, 90%, or 100% reduction in the severity of an established disease, condition, or symptom of the disease or condition. For example, a method for treating a disease is considered to be a treatment if there is a 10% reduction in one or more symptoms of the disease in a subject as compared to a control. Thus the reduction can be a 10%, 20%, 30%, 40%, 50%, 60%, 70%, 80%, 90%, 100%, or any percent reduction in between 10% and 100% as compared to native or control levels. It is understood that treatment does not necessarily refer to a cure or complete ablation of the disease, condition, or symptoms of the disease or condition. Further, as used herein, references to decreasing, reducing, or inhibiting include a change of 10%, 20%, 30%, 40%, 50%, 60%, 70%, 80%, 90% or greater as compared to a control level and such terms can include but do not necessarily include complete elimination. Symptoms of disease, such as for arthritis, include synovitis, bone erosion, and cartilage depletion.

As used herein, the terms “PTPRS de-clustering agent” and the like refer to an agent (e.g., small molecules, proteins including antibodies, and the like) capable of causing a reduction in the level of dimerization, oligomerization or clustering of PTPRS proteins. Without wishing to be bound by any theory, it is believed that clustering of PTPRS by HS can give rise to an inactive dimeric or other oligomeric form. Accordingly, the action of a PTPRS de-clustering agent results in monomeric PTPRS which regains activity relative to the clustered (e.g., dimerized or oligomerized) form of PTPRS. Optionally, the PTPRS de-clustering agent is a non-enzymatic recombinant protein comprising an amino acid sequence of an extracellular domain of PTPRS or a subsequence, portion, homologue, variant or derivative thereof, as described herein. Optionally, the non-enzymatic recombinant protein is the extracellular domain of PTPRS. Without being limited to any particular theory, the extracellular domain of PTPRS or portions thereof displaces PTPRS from HS. This can activate PTPRS and lead to dephosphorylation of beta-catenin and other substrates (such as ezrin in synoviocytes) and inhibition of downstream FLS invasiveness and pro-inflammatory actions. This is supported by the examples and data provided herein. Optionally, the PTPRS de-clustering agent is an anti-PRPRS antibody or fragment thereof. Optionally, the PTPRS de-clustering agent is an anti-PTPRS aptamer. Optionally, the PTPRS de-clustering agent binds heparan sulfate. Optionally, the PTPRS de-clustering agent is an anti-heparan sulfate antibody or fragment thereof. Optionally, the PTPRS de-clustering agent is an anti-heparan sulfate aptamer. Optionally, the PTPRS de-clustering agent is not chondroitin sulfate. The PTPRS de-clustering agent is, optionally, not a chondroitin sulfate mimetic or an agent that has the same or similar mechanism of action as chondroitin sulfate. In other embodiments, the PTPRS de-clustering agent is a chondroitin sulfate mimetic.

As used herein, the term “non-enzymatic recombinant protein” refers to a recombinant protein that does not have enzymatic activity (e.g., the protein does not function as a biological catalyst). Thus, in some embodiments, the non-enzymatic recombinant proteins comprising an amino acid sequence of an extracellular domain of PTPRS include only extracellular domain portions of the PTPRS and not the enzymatic portions of the PTPRS. In embodiments, the non-enzymatic recombinant proteins comprising an amino acid sequence of an extracellular domain of PTPRS include only extracellular domain portions of the PTPRS and not the enzymatic portions of the PTPRS or the transmembrane portions of the PTPRS. In some embodiments, the non-enzymatic recombinant proteins comprising an amino acid sequence of an extracellular domain of PTPRS include two or more extracellular domain of PTPRS linked together (e.g. linked by an amino acid linker such as an amino acid linker of at least 2, at least 3, at least 5, at least 10, about 2 to 50 or 100 amino acids, about 3 to 50 or 100 amino acids, or about 2, 3, 4 5, 6, 7, 8, 9, 10, 20, 30, 40, 50, 60, 70, 80, 90, or 100 amino acids wherein the amino acid sequence is designed to not interfere with extracellular domain of PTPRS ligand binding). The term “extracellular domain of PTPRS” can include subsequences, portions, homologues, variants or derivatives of the extracellular domain of PTPRS. Thus, the non-enzymatic recombinant protein can comprise a portion of the extracellular domain of PTPRS, e.g., the protein comprises one or more immunoglobulin-like domains of PTPRS, e.g., SEQ ID NO:1, SEQ ID NO:2, SEQ ID NO:3, SEQ ID NO:5, SEQ ID NO:6 and/or SEQ ID NO:7, or subsequences, portions, homologues, variants or derivatives thereof. The extracellular domain of PTPRS is typically capable of binding (e.g specifically binding) to a PTPRS ligand such as heparan sulfate. Optionally, the extracellular domain of PTPRS comprises one or more of PTPRS immunoglobulin-like domain 1 (Ig1), immunoglobulin-like domain 2 (Ig2) and immunoglobulin-like domain 2 (Ig3), or a subsequence, portion, homologue, variant or derivative thereof. Optionally, the extracellular domain of PTPRS comprises one or both of PTPRS immunoglobulin-like domain 1 (Ig1) and immunoglobulin-like domain 2 (Ig2) or a subsequence, portion, homologue, variant or derivative thereof.

Optionally, the protein comprises Ig1 amino acid residues 39 to 124 of SEQ ID NO:4, or a subsequence, portion, homologue, variant or derivative thereof. Optionally, the protein comprises an amino acid sequence set forth as: EEPRFIKEPKDQIGVSGGVASFVCQATGDPKPRVTWNKKGKKVNSQRFETIEFDESA GAVLRIQPLRTPRDENVYECVAQNSVGEITVHAKLTVLRE (SEQ ID NO:1) or set forth as SEQ ID NO:5, or a subsequence, portion, homologue, variant or derivative thereof.

Optionally, the protein comprises Ig2 amino acid residues 152 to 233 of SEQ ID NO:4, or a subsequence, portion, homologue, variant or derivative thereof. Optionally, the protein comprises an amino acid sequence set forth as: DQLPSGFPNIDMGPQLKVVERTRTATMLCAASGNPDPEITWFKDFLPVDPSASNGRI KQLRSETFESTPIRGALQIESSEETDQGKYECVATNSAGVRYSSPANLYVRVRRVA (SEQ ID NO:2) or set forth as SEQ ID NO:6, or a subsequence, portion, homologue, variant or derivative thereof.

Optionally, the protein comprises Ig3 amino acid residues 259-327 of SEQ ID NO:4, or a subsequence, portion, homologue, variant or derivative thereof. Optionally, the protein comprises an amino acid sequence set forth as: PRFSILPMSHEIMPGGNVNITCVAVGSPMPYVKWMQGAEDLTPEDDMPVGRNVLEL TDVKDSANYTCVAMSSLGVIEAVAQITVKSLPKA (SEQ ID NO:3), or set forth as SEQ ID NO:7, or a subsequence, portion, homologue, variant or derivative thereof.

In some embodiments, the non-enzymatic recombinant protein comprising an amino acid sequence of an extracellular domain of PTPRS or portion thereof lacks a transmembrane domain and/or lacks an intracellular domain. In some embodiments, the non-enzymatic recombinant protein comprising an amino acid sequence of an extracellular domain of PTPRS or portion thereof lacks a transmembrane domain. In some embodiments, the non-enzymatic recombinant protein comprising an amino acid sequence of an extracellular domain of PTPRS or portion thereof lacks an intracellular domain.

Optionally, the provided PTPRS de-clustering agent binds (e.g. specifically binds) heparan sulfate. Optionally, the PTPRS de-clustering agent prevents oligomerization or clustering of PTPRS proteins; for example, the PTPRS de-clustering agent prevents dimerization of PTPRS proteins. Optionally, the PTPRS de-clustering agent modulates PTPRS activity; for example, the PTPRS de-clustering agent increases the phosphatase activity of PTPRS. Optionally, the PTPRS de-clustering agent removes phosphatase from its substrate or from its site of action.

Provided herein are compositions including the agents provided herein. Provided herein are pharmaceutical compositions including a PTPRS de-clustering agent, a TNF inhibitor, and a pharmaceutically acceptable excipient. Provided herein are pharmaceutical compositions including a PTPRS de-clustering agent, an IL-6 inhibitor, and a pharmaceutically acceptable excipient. Provided compositions can include additional agents. The provided compositions are, optionally, suitable for formulation and administration in vitro or in vivo. Optionally, the compositions comprise one or more of the provided agents and a pharmaceutically acceptable carrier. Suitable carriers and their formulations are described in Remington: The Science and Practice of Pharmacy, 21st Edition, David B. Troy, ed., Lippicott Williams & Wilkins (2005). By pharmaceutically acceptable carrier is meant a material that is not biologically or otherwise undesirable, i.e., the material is administered to a subject without causing undesirable biological effects or interacting in a deleterious manner with the other components of the pharmaceutical composition in which it is contained. If administered to a subject, the carrier is optionally selected to minimize degradation of the active ingredient and to minimize adverse side effects in the subject.

In embodiments, the PTPRS de-clustering agent and the TNF or IL-6 inhibitor are administered in a combined synergistic amount. A “combined synergistic amount” as used herein refers to the sum of a first amount (e.g., an amount of an PTPRS de-clustering agent) and a second amount (e.g., an amount of a TNF or IL-6 inhibitor) that results in a synergistic effect (i.e. an effect greater than an additive effect). Therefore, the terms “synergy”, “synergism”, “synergistic”, “combined synergistic amount”, and “synergistic therapeutic effect” which are used herein interchangeably, refer to a measured effect of compounds administered in combination where the measured effect is greater than the sum of the individual effects of each of the compounds administered alone as a single agent.

In embodiments, a synergistic amount may be about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99% of the amount of the PTPRS de-clustering agent when used separately from the TNF or IL-6 inhibitor. In embodiments, a synergistic amount may be about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99% of the amount of the TNF or IL-6 inhibitor when used separately from the PTPRS de-clustering agent.

The synergistic effect may be PTPRS de-clustering activity decreasing effect and/or a TNF or IL-6 activity decreasing effect. In embodiments, synergy between the PTPRS de-clustering agent and the TNF or IL-6 inhibitor may result in about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% greater decrease (e.g., decrease of PTPRS de-clustering activity or decrease of TNF or IL-6 activity) than the sum of the decrease of the PTPRS de-clustering agent or the TNF or IL-6 when used individually and separately. In embodiments, synergy between the PTPRS de-clustering agent and the TNF or IL-6 inhibitor may result in 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% greater inhibition of the PTPRS de-clustering and/or the TNF or IL-6 activity than the sum of the inhibition of the PTPRS de-clustering agent or the TNF or IL-6 inhibitor when used individually and separately.

The PTPRS de-clustering agent and the TNF or IL-6 inhibitor may be administered in combination either concomitantly (e.g., as a mixture), separately but simultaneously (e.g., via separate intravenous lines) or sequentially (e.g., one agent is administered first followed by administration of the second agent). Thus, the term combination is used to refer to concomitant, simultaneous or sequential administration of the PTPRS de-clustering agent and the TNF or IL-6 inhibitor. In embodiments, where the PTPRS de-clustering agent and the TNF or IL-6 inhibitor are administered sequentially, the PTPRS de-clustering agent is administered at a first time point and the TNF or IL-6 inhibitor is administered at a second time point, wherein the first time point precedes the second time point. The course of treatment is best determined on an individual basis depending on the particular characteristics of the subject and the type of treatment selected. The treatment, such as those disclosed herein, can be administered to the subject on a daily, twice daily, bi-weekly, monthly or any applicable basis that is therapeutically effective. The treatment can be administered alone or in combination with any other treatment disclosed herein or known in the art. The additional treatment can be administered simultaneously with the first treatment, at a different time, or on an entirely different therapeutic schedule (e.g., the first treatment can be daily, while the additional treatment is weekly). Thus, in embodiments, the PTPRS de-clustering agent and the TNF or IL-6 inhibitor are administered simultaneously or sequentially.

In embodiments, the PTPRS de-clustering agent is administered at a first time point and the TNF or IL-6 inhibitor is administered at a second time point, wherein the first time point precedes the second time point. In embodiments, the second time point is within less than about 120, 90, 60, 50, 40, 30, 20, 19, 18, 17, 16, 15, 14, 13, 12, 10, 11, 9, 8, 7, 6, 5, 4, 3, 2 or 1 days from the first time point. In embodiments, the second time point is within less than about 120 days from the first time point. In embodiments, the second time point is within less than about 90 days from the first time point. In embodiments, the second time point is within less than about 60 days from the first time point. In embodiments, the second time point is within less than about 50 days from the first time point. In embodiments, the second time point is within less than about 40 days from the first time point. In embodiments, the second time point is within less than about 30 days from the first time point. In embodiments, the second time point is within less than about 20 days from the first time point.

In embodiments, the second time point is within less than about 19 days from the first time point. In embodiments, the second time point is within less than about 18 days from the first time point. In embodiments, the second time point is within less than about 17 days from the first time point. In embodiments, the second time point is within less than about 16 days from the first time point. In embodiments, the second time point is within less than about 15 days from the first time point. In embodiments, the second time point is within less than about 14 days from the first time point. In embodiments, the second time point is within less than about 13 days from the first time point. In embodiments, the second time point is within less than about 12 days from the first time point. In embodiments, the second time point is within less than about 11 days from the first time point. In embodiments, the second time point is within less than about 10 days from the first time point. In embodiments, the second time point is within less than about 9 days from the first time point. In embodiments, the second time point is within less than about 8 days from the first time point. In embodiments, the second time point is within less than about 7 days from the first time point. In embodiments, the second time point is within less than about 6 days from the first time point. In embodiments, the second time point is within less than about 5 days from the first time point. In embodiments, the second time point is within less than about 4 days from the first time point. In embodiments, the second time point is within less than about 3 days from the first time point. In embodiments, the second time point is within less than about 2 days from the first time point. In embodiments, the second time point is within less than about 1 day from the first time point.

In embodiments, the second time point is within about 8, 10 or 12 days from the first time point. In embodiments, the second time point is within about 8, days from the first time point. In embodiments, the second time point is within about 10 days from the first time point. In embodiments, the second time point is within about 12 days from the first time point. In embodiments, the TNF or IL-6 inhibitor and the PTPRS de-clustering agent are simultaneously administered at the second time point. In embodiments, the TNF or IL-6 inhibitor and the PTPRS de-clustering agent are concomitantly administered at the second time point. In embodiments, the TNF or IL-6 inhibitor is administered at the second time point and the PTPRS de-clustering agent is not administered at the second time point.

In embodiments, the TNF or IL-6 inhibitor is administered at a first time point and the PTPRS de-clustering agent is administered at a second time point, wherein the first time point precedes the second time point. In embodiments, the second time point is within less than about 120, 90, 60, 50, 40, 30, 20, 19, 18, 17, 16, 15, 14, 13, 12, 10, 11, 9, 8, 7, 6, 5, 4, 3, 2 or 1 days from the first time point. In embodiments, the second time point is within less than about 120 days from the first time point. In embodiments, the second time point is within less than about 90 days from the first time point. In embodiments, the second time point is within less than about 60 days from the first time point. In embodiments, the second time point is within less than about 50 days from the first time point. In embodiments, the second time point is within less than about 40 days from the first time point. In embodiments, the second time point is within less than about 30 days from the first time point. In embodiments, the second time point is within less than about 20 days from the first time point.

In embodiments, the second time point is within less than about 19 days from the first time point. In embodiments, the second time point is within less than about 18 days from the first time point. In embodiments, the second time point is within less than about 17 days from the first time point. In embodiments, the second time point is within less than about 16 days from the first time point. In embodiments, the second time point is within less than about 15 days from the first time point. In embodiments, the second time point is within less than about 14 days from the first time point. In embodiments, the second time point is within less than about 13 days from the first time point. In embodiments, the second time point is within less than about 12 days from the first time point. In embodiments, the second time point is within less than about 11 days from the first time point. In embodiments, the second time point is within less than about 10 days from the first time point. In embodiments, the second time point is within less than about 9 days from the first time point. In embodiments, the second time point is within less than about 8 days from the first time point. In embodiments, the second time point is within less than about 7 days from the first time point. In embodiments, the second time point is within less than about 6 days from the first time point. In embodiments, the second time point is within less than about 5 days from the first time point. In embodiments, the second time point is within less than about 4 days from the first time point. In embodiments, the second time point is within less than about 3 days from the first time point. In embodiments, the second time point is within less than about 2 days from the first time point. In embodiments, the second time point is within less than about 1 day from the first time point.

In embodiments, the second time point is within about 8, 10 or 12 days from the first time point. In embodiments, the second time point is within about 8, days from the first time point. In embodiments, the second time point is within about 10 days from the first time point. In embodiments, the second time point is within about 12 days from the first time point. In embodiments, the TNF or IL-6 inhibitor and the PTPRS de-clustering agent are simultaneously administered at the second time point. In embodiments, the TNF or IL-6 inhibitor and the PTPRS de-clustering agent are concomitantly administered at the second time point. In embodiments, the PTPRS de-clustering agent is administered at the second time point and the TNF or IL-6 inhibitor is not administered at the second time point.

According to the methods provided herein, the subject is administered an effective amount of two or more of the agents (e.g., a PTPRS de-clustering agent and a TNF or IL-6 inhibitor) provided herein. An “effective amount” is an amount sufficient to accomplish a stated purpose (e.g. achieve the effect for which it is administered, treat a disease (e.g., RA), induce PTPRS activity, reduce one or more symptoms of a disease or condition). An example of an “effective amount” is an amount sufficient to contribute to the treatment, prevention, or reduction of a symptom or symptoms of a disease (e.g., cancer), which could also be referred to as a “therapeutically effective amount.” A “reduction” of a symptom or symptoms (and grammatical equivalents of this phrase) means decreasing of the severity or frequency of the symptom(s), or elimination of the symptom(s). Guidance can be found in the literature for appropriate dosages for given classes of pharmaceutical products. For example, for the given parameter, a therapeutically effective amount will show an increase or decrease of at least 5%, 10%, 15%, 20%, 25%, 40%, 50%, 60%, 75%, 80%, 90%, or at least 100%. Efficacy can also be expressed as “-fold” increase or decrease. For example, a therapeutically effective amount can have at least a 1.2-fold, 1.5-fold, 2-fold, 5-fold, or more effect over a control. The exact amounts will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins)

In embodiments, the PTPRS de-clustering agent is administered at an amount of about 0.5 mg/kg, 1 mg/kg, 2 mg/kg, 3 mg/kg, 4 mg/kg, 5 mg/kg, 10 mg/kg, 20 mg/kg, 30 mg/kg, 40 mg/kg, 50 mg/kg, 60 mg/kg, 70 mg/kg, 80 mg/kg, 90 mg/kg, 100 mg/kg, 200 mg/kg or 300 mg/kg. In embodiments, the PTPRS de-clustering agent is administered at an amount of about 0.5 mg/kg. In embodiments, the PTPRS de-clustering agent is administered at an amount of about 1 mg/kg. In embodiments, the PTPRS de-clustering agent is administered at an amount of about 5 mg/kg. In embodiments, the PTPRS de-clustering agent is administered at an amount of about 10 mg/kg. In embodiments, the PTPRS de-clustering agent is administered at an amount of about 20 mg/kg. In embodiments, the PTPRS de-clustering agent is administered at an amount of about 30 mg/kg. In embodiments, the PTPRS de-clustering agent is administered at an amount of about 40 mg/kg. In embodiments, the PTPRS de-clustering agent is administered at an amount of about 50 mg/kg. In embodiments, the PTPRS de-clustering agent is administered at an amount of about 60 mg/kg. In embodiments, the PTPRS de-clustering agent is administered at an amount of about 70 mg/kg. In embodiments, the PTPRS de-clustering agent is administered at an amount of about 80 mg/kg. In embodiments, the PTPRS de-clustering agent is administered at an amount of about 90 mg/kg. In embodiments, the PTPRS de-clustering agent is administered at an amount of about 100 mg/kg. In embodiments, the PTPRS de-clustering agent is administered at an amount of about 200 mg/kg. In embodiments, the PTPRS de-clustering agent is administered at an amount of about 300 mg/kg. It is understood that where the amount is referred to as “mg/kg”, the amount is milligram per kilogram body weight of the subject being administered with the PTPRS de-clustering agent.

In embodiments, the PTPRS de-clustering agent is administered at an amount of about 0.5 mg/kg, 1 mg/kg, 5 mg/kg, 10 mg/kg, 20 mg/kg, 30 mg/kg, 40 mg/kg, 50 mg/kg, 60 mg/kg, 70 mg/kg, 80 mg/kg, 90 mg/kg, 100 mg/kg, 200 mg/kg or 300 mg/kg. In embodiments, the PTPRS de-clustering agent is administered at an amount of about 1 mg/kg. In embodiments, the PTPRS de-clustering agent is administered at an amount of about 1 mg/kg to 2 mg/kg. In embodiments, the PTPRS de-clustering agent is administered at an amount of about 1 mg/kg to 3 mg/kg. In embodiments, the PTPRS de-clustering agent is administered at an amount of about 1 mg/kg to 4 mg/kg. In embodiments, the PTPRS de-clustering agent is administered at an amount of about 1 mg/kg to 5 mg/kg.

In embodiments, the PTPRS de-clustering agent is administered at an amount of about 10 mg BID, 20 mg BID, 30 mg BID, 40 mg BID, 50 mg BID, 60 mg BID, 70 mg BID, 80 mg BID, 90 mg BID, 100 mg BID, 110 mg BID, 120 mg BID, 130 mg BID, 140 mg BID, 150 mg BID, 160 mg BID, 170 mg BID, 180 mg BID, 190 mg BID, 200 mg BID, 210 mg BID, 220 mg BID, 230 mg BID, 240 mg BID, 250 mg BID, 260 mg BID, 270 mg BID, 280 mg BID, 290 mg BID, or 300 mg BID. In embodiments, the PTPRS de-clustering agent is administered at an amount of about 10 mg BID. In embodiments, the PTPRS de-clustering agent is administered at an amount of about 20 mg BID. In embodiments, the PTPRS de-clustering agent is administered at an amount of about 30 mg BID. In embodiments, the PTPRS de-clustering agent is administered at an amount of about 40 mg BID. In embodiments, the PTPRS de-clustering agent is administered at an amount of about 50 mg BID. In embodiments, the PTPRS de-clustering agent is administered at an amount of about 60 mg BID. In embodiments, the PTPRS de-clustering agent is administered at an amount of about 70 mg BID. In embodiments, the PTPRS de-clustering agent is administered at an amount of about 80 mg BID. In embodiments, the PTPRS de-clustering agent is administered at an amount of about 90 mg BID. In embodiments, the PTPRS de-clustering agent is administered at an amount of about 100 mg BID. It is understood that where the amount is referred to as “BID” which stands for “bis in die”, the amount is administered twice a day.

In embodiments, the PTPRS de-clustering agent is administered at an amount of about 110 mg BID. In embodiments, the PTPRS de-clustering agent is administered at an amount of about 120 mg BID. In embodiments, the PTPRS de-clustering agent is administered at an amount of about 130 mg BID. In embodiments, the PTPRS de-clustering agent is administered at an amount of about 140 mg BID. In embodiments, the PTPRS de-clustering agent is administered at an amount of about 150 mg BID. In embodiments, the PTPRS de-clustering agent is administered at an amount of about 160 mg BID. In embodiments, the PTPRS de-clustering agent is administered at an amount of about 170 mg BID. In embodiments, the PTPRS de-clustering agent is administered at an amount of about 180 mg BID. In embodiments, the PTPRS de-clustering agent is administered at an amount of about 190 mg BID. In embodiments, the PTPRS de-clustering agent is administered at an amount of about 200 mg BID. It is understood that where the amount is referred to as “BID” which stands for “bis in die”, the amount is administered twice a day.

In embodiments, the PTPRS de-clustering agent is administered at an amount of about 210 mg BID. In embodiments, the PTPRS de-clustering agent is administered at an amount of about 220 mg BID. In embodiments, the PTPRS de-clustering agent is administered at an amount of about 230 mg BID. In embodiments, the PTPRS de-clustering agent is administered at an amount of about 240 mg BID. In embodiments, the PTPRS de-clustering agent is administered at an amount of about 250 mg BID. In embodiments, the PTPRS de-clustering agent is administered at an amount of about 260 mg BID. In embodiments, the PTPRS de-clustering agent is administered at an amount of about 270 mg BID. In embodiments, the PTPRS de-clustering agent is administered at an amount of about 280 mg BID. In embodiments, the PTPRS de-clustering agent is administered at an amount of about 290 mg BID. In embodiments, the PTPRS de-clustering agent is administered at an amount of about 300 mg BID. It is understood that where the amount is referred to as “BID” which stands for “bis in die”, the amount is administered twice a day.

In embodiments, the PTPRS de-clustering agent is administered at an amount of about 10 mg QD, 20 mg QD, 30 mg QD, 40 mg QD, 50 mg QD, 60 mg QD, 70 mg QD, 80 mg QD, 90 mg QD, 100 mg QD, 110 mg QD, 120 mg QD, 130 mg QD, 140 mg QD, 150 mg QD, 160 mg QD, 170 mg QD, 180 mg QD, 190 mg QD, 200 mg QD, 210 mg QD, 220 mg QD, 230 mg QD, 240 mg QD, 250 mg QD, 260 mg QD, 270 mg QD, 280 mg QD, 290 mg QD, or 300 mg QD. In embodiments, the PTPRS de-clustering agent is administered at an amount of about 10 mg QD. In embodiments, the PTPRS de-clustering agent is administered at an amount of about 20 mg QD. In embodiments, the PTPRS de-clustering agent is administered at an amount of about 30 mg QD. In embodiments, the PTPRS de-clustering agent is administered at an amount of about 40 mg QD. In embodiments, the PTPRS de-clustering agent is administered at an amount of about 50 mg QD. In embodiments, the PTPRS de-clustering agent is administered at an amount of about 60 mg QD. In embodiments, the PTPRS de-clustering agent is administered at an amount of about 70 mg QD. In embodiments, the PTPRS de-clustering agent is administered at an amount of about 80 mg QD. In embodiments, the PTPRS de-clustering agent is administered at an amount of about 90 mg QD. In embodiments, the PTPRS de-clustering agent is administered at an amount of about 100 mg QD. It is understood that where the amount is referred to as “QD” which stands for “quaque die”, the amount is administered once a day.

In embodiments, the PTPRS de-clustering agent is administered at an amount of about 110 mg QD. In embodiments, the PTPRS de-clustering agent is administered at an amount of about 120 mg QD. In embodiments, the PTPRS de-clustering agent is administered at an amount of about 130 mg QD. In embodiments, the PTPRS de-clustering agent is administered at an amount of about 140 mg QD. In embodiments, the PTPRS de-clustering agent is administered at an amount of about 150 mg QD. In embodiments, the PTPRS de-clustering agent is administered at an amount of about 160 mg QD. In embodiments, the PTPRS de-clustering agent is administered at an amount of about 170 mg QD. In embodiments, the PTPRS de-clustering agent is administered at an amount of about 180 mg QD. In embodiments, the PTPRS de-clustering agent is administered at an amount of about 190 mg QD. In embodiments, the PTPRS de-clustering agent is administered at an amount of about 200 mg QD. It is understood that where the amount is referred to as “QD” which stands for “quaque die”, the amount is administered once a day.

In embodiments, the PTPRS de-clustering agent is administered at an amount of about 210 mg QD. In embodiments, the PTPRS de-clustering agent is administered at an amount of about 220 mg QD. In embodiments, the PTPRS de-clustering agent is administered at an amount of about 230 mg QD. In embodiments, the PTPRS de-clustering agent is administered at an amount of about 240 mg QD. In embodiments, the PTPRS de-clustering agent is administered at an amount of about 250 mg QD. In embodiments, the PTPRS de-clustering agent is administered at an amount of about 260 mg QD. In embodiments, the PTPRS de-clustering agent is administered at an amount of about 270 mg QD. In embodiments, the PTPRS de-clustering agent is administered at an amount of about 280 mg QD. In embodiments, the PTPRS de-clustering agent is administered at an amount of about 290 mg QD. In embodiments, the PTPRS de-clustering agent is administered at an amount of about 300 mg QD. It is understood that where the amount is referred to as “QD” which stands for “quaque die”, the amount is administered once a day.

The PTPRS de-clustering agent and TNF or IL-6 inhibitor may be administered at an amount as provided herein for the patient's lifetime, for a year, a month or a week. The PTPRS de-clustering agent and TNF or IL-6 inhibitor may be administered at an amount as provided herein daily, weekly or monthly. The PTPRS de-clustering agent and TNF or IL-6 inhibitor may be administered at an amount as provided herein on 28 consecutive days. The PTPRS de-clustering agent and TNF or IL-6 inhibitor may be administered at an amount as provided herein on 14 consecutive days. In embodiments, the PTPRS de-clustering agent and TNF or IL-6 inhibitor is administered BID or QD. In further embodiments, the PTPRS de-clustering agent and the TNF or IL-6 inhibitor are administered simultaneously on 365, 28, or 14 consecutive days. In other further embodiments, the PTPRS de-clustering agent and the TNF or IL-6 inhibitor are administered simultaneously on 14 consecutive days.

In some embodiments, the PTPRS de-clustering agent or TNF or IL-6 inhibitor are administered weekly or monthly.

In some embodiments, the TNF inhibitor is Etanercept (Enbrel) or its biosimilar. In some embodiments, the second amount is below 50 mg. In some embodiments, the second amount is 10%, 20%, 30%, 40%, 50%, 60%, 70%, 80%, 90%, 95%, 98% or 99% below 50 mg.

In some embodiments, the TNF inhibitor is Adalimumab (Humira) or its biosimilar. In some embodiments, the second amount is below 40 mg. In some embodiments, the second amount is 10%, 20%, 30%, 40%, 50%, 60%, 70%, 80%, 90%, 95%, 98% or 99% below 40 mg.

In some embodiments, the TNF inhibitor is Infliximab (Remicade) or its biosimilar. In some embodiments, the second amount is below 3 mg/kg. In some embodiments, the second amount is 10%, 20%, 30%, 40%, 50%, 60%, 70%, 80%, 90%, 95%, 98% or 99% below 3 mg/kg.

In some embodiments, the TNF inhibitor is Golimumab (Simponi) or its biosimilar. In some embodiments, the second amount is below 50 mg. In some embodiments, the second amount is 10%, 20%, 30%, 40%, 50%, 60%, 70%, 80%, 90%, 95%, 98% or 99% below 50 mg.

In some embodiments, the TNF inhibitor is Certolizumab, Certolizumab pegol (Cimzia) or its biosimilar. In some embodiments, the second amount is below 200 mg. In some embodiments, the second amount is 10%, 20%, 30%, 40%, 50%, 60%, 70%, 80%, 90%, 95%, 98% or 99% below 200 mg.

Provided herein are pharmaceutical compositions that include a first amount of a PTPRS de-clustering agent and a second amount of an IL-6 inhibitor, wherein the second amount is below a therapeutically effective level of the IL-6 inhibitor. The second amount can be 10%, 20%, 30%, 40%, 50%, 60%, 70%, 80%, 90%, 95%, 98% or 99% below the therapeutically effective level of the IL-6 inhibitor.

In some embodiments, the IL-6 inhibitor is Tocilizumab (Atlizumab) or its biosimilar. In some embodiments, the drug is administered by IV infusion and the second amount is below 4 mg/kg. In some embodiments, the second amount is 10%, 20%, 30%, 40%, 50%, 60%, 70%, 80%, 90%, 95%, 98% or 99% below 4 mg/kg. In some embodiments, the drug is administered SC, and the second amount is below 162 mg. In some embodiments, the second amount is administered SC and is 10%, 20%, 30%, 40%, 50%, 60%, 70%, 80%, 90%, 95%, 98% or 99% below 162 mg. For treatment of RA by SC, in patients less than 100 kg, 162 mg subcutaneously every other week, followed by an increase to every week based on clinical response.

In some embodiments, the IL-6 inhibitor is Sarilumab (Kevzara) or its biosimilar. In some embodiments, the second amount is below 100 mg. In some embodiments, the second amount is 10%, 20%, 30%, 40%, 50%, 60%, 70%, 80%, 90%, 95%, 98% or 99% below 100 mg. For treatment of RA by SC, in patients less than 100 kg 100 mg subcutaneously once daily.

Provided herein are any of the above pharmaceutical compositions, wherein the first amount is below a therapeutically effective level of the PTPRS de-clustering agent. In some embodiments, the PTPRS de-clustering agent includes one or both of PTPRS immunoglobulin-like domain 1 (Ig1) and immunoglobulin-like domain 2 (Ig2). In some embodiments, the PTPRS de-clustering agent includes Ig1 amino acid residues 30 to 127 of SEQ ID NO:4 or amino acid residues 30-127 of SEQ ID NO:8. In some embodiments, the PTPRS de-clustering agent includes an amino acid sequence set forth as: EEPRFIKEPKDQIGVSGGVASFVCQATGDPKPRVTWNKKGKKVNSQRFETIEFDESA GAVLRIQPLRTPRDENVYECVAQNSVGEITVHAKLTVLRE (SEQ ID NO:1) or as set forth in SEQ ID NO:5. In some embodiments, the PTPRS de-clustering agent includes Ig2 amino acid residues 128 to 231 of SEQ ID NO:4 or amino acid residues 128-244 of SEQ ID NO:8. In some embodiments, the PTPRS de-clustering agent includes an amino acid sequence set forth as: DQLPSGFPNIDMGPQLKVVERTRTATMLCAASGNPDPEITWFKDFLPVDPSASNGRI KQLRSETFESTPIRGALQIESSEETDQGKYECVATNSAGVRYSSPANLYVRVRRVA (SEQ ID NO:2) or as set forth in SEQ ID NO:6. In some embodiments, the PTPRS de-clustering agent includes Ig3 amino acid residues 232-321 of SEQ ID NO:4 or amino acid residues 245-334 of SEQ ID NO:8. In some embodiments, the PTPRS de-clustering agent includes an amino acid sequence set forth as: PRFSILPMSHEIMPGGNVNITCVAVGSPMPYVKWMQGAEDLTPEDDMPVGRNVLEL TDVKDSANYTCVAMSSLGVIEAVAQITVKSLPKA (SEQ ID NO:3) or as set forth in SEQ ID NO:7. In some embodiments, the amino acid sequence of the PTPRS de-clustering agent can have about 60% identity, preferably 65%, 70%, 75%, 80%, 85%, 90%, 91%, 92%, 93%, 94%, 95%, 96%, 97%, 98%, 99%, or higher to the above recited sequences.

In some embodiments, the PTPRS de-clustering agent binds heparan sulfate. In some embodiments, the PTPRS de-clustering agent lacks a transmembrane domain. In some embodiments, the PTPRS de-clustering agent lacks an intracellular domain.

In some embodiments, provided herein are pharmaceutical compositions of any one of the above combinations of a first amount of PTPRS de-clustering agent and a second amount of a TNF or an IL-6 inhibitor are present in a combined synergistic amount.

The term “pharmaceutically acceptable salts” or “pharmaceutically acceptable carrier” is meant to include salts of the active compounds which are prepared with relatively nontoxic acids or bases, depending on the particular substituents found on the compounds described herein. When compounds of the present application contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present application contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, e.g., Berge et al., Journal of Pharmaceutical Science 66:1-19 (1977)). Other pharmaceutically acceptable carriers known to those of skill in the art are suitable for compositions of the present application.

The compositions for administration will commonly comprise an agent as described herein dissolved in a pharmaceutically acceptable carrier, preferably an aqueous carrier. A variety of aqueous carriers can be used, e.g., buffered saline and the like. These solutions are sterile and generally free of undesirable matter. These compositions may be sterilized by conventional, well known sterilization techniques. The compositions may contain pharmaceutically acceptable auxiliary substances as required to approximate physiological conditions such as pH adjusting and buffering agents, toxicity adjusting agents and the like, for example, sodium acetate, sodium chloride, potassium chloride, calcium chloride, sodium lactate and the like. The concentration of active agent in these formulations can vary widely, and will be selected primarily based on fluid volumes, viscosities, body weight and the like in accordance with the particular mode of administration selected and the subject's needs.

Solutions of the active compounds as free base or pharmacologically acceptable salt can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations can contain a preservative to prevent the growth of microorganisms.

Pharmaceutical compositions can be delivered via intranasal or inhalable solutions or sprays, aerosols or inhalants. Nasal solutions can be aqueous solutions designed to be administered to the nasal passages in drops or sprays. Nasal solutions can be prepared so that they are similar in many respects to nasal secretions. Thus, the aqueous nasal solutions usually are isotonic and slightly buffered to maintain a pH of 5.5 to 6.5. In addition, antimicrobial preservatives, similar to those used in ophthalmic preparations and appropriate drug stabilizers, if required, may be included in the formulation. Various commercial nasal preparations are known and can include, for example, antibiotics and antihistamines.

Oral formulations can include excipients as, for example, pharmaceutical grades of mannitol, lactose, starch, magnesium stearate, sodium saccharine, cellulose, magnesium carbonate and the like. These compositions take the form of solutions, suspensions, tablets, pills, capsules, sustained release formulations or powders. In some embodiments, oral pharmaceutical compositions will comprise an inert diluent or assimilable edible carrier, or they may be enclosed in hard or soft shell gelatin capsule, or they may be compressed into tablets, or they may be incorporated directly with the food of the diet. For oral therapeutic administration, the active compounds may be incorporated with excipients and used in the form of ingestible tablets, buccal tablets, troches, capsules, elixirs, suspensions, syrups, wafers, and the like. Such compositions and preparations should contain at least 0.1% of active compound. The percentage of the compositions and preparations may, of course, be varied and may conveniently be between about 2 to about 75% of the weight of the unit, or preferably between 25-60%. The amount of active compounds in such compositions is such that a suitable dosage can be obtained

For parenteral administration in an aqueous solution, for example, the solution should be suitably buffered and the liquid diluent first rendered isotonic with sufficient saline or glucose. Aqueous solutions, in particular, sterile aqueous media, are especially suitable for intravenous, intramuscular, subcutaneous and intraperitoneal administration. For example, one dosage could be dissolved in 1 ml of isotonic NaCl solution and either added to 1000 ml of hypodermoclysis fluid or injected at the proposed site of infusion

Sterile injectable solutions can be prepared by incorporating the active compounds or constructs in the required amount in the appropriate solvent followed by filtered sterilization. Generally, dispersions are prepared by incorporating the various sterilized active ingredients into a sterile vehicle which contains the basic dispersion medium. Vacuum-drying and freeze-drying techniques, which yield a powder of the active ingredient plus any additional desired ingredients, can be used to prepare sterile powders for reconstitution of sterile injectable solutions. The preparation of more, or highly, concentrated solutions for direct injection is also contemplated. DMSO can be used as solvent for extremely rapid penetration, delivering high concentrations of the active agents to a small area.

The formulations of compounds can be presented in unit-dose or multi-dose sealed containers, such as ampules and vials. Thus, the composition can be in unit dosage form. In such form the preparation is subdivided into unit doses containing appropriate quantities of the active component. Thus, the compositions can be administered in a variety of unit dosage forms depending upon the method of administration. For example, unit dosage forms suitable for oral administration include, but are not limited to, powder, tablets, pills, capsules and lozenges.

Compositions can be formulated to provide quick, sustained or delayed release after administration by employing procedures known in the art. Certain carriers may be more preferable depending upon, for instance, the route of administration and concentration of composition being administered. Suitable formulations for use in the provided compositions can be found in Remington: The Science and Practice of Pharmacy, 21st Edition, David B. Troy, ed., Lippicott Williams & Wilkins (2005).

Provided herein are kits comprising one or more of the provided compositions and instructions for use. Optionally, the kit comprises one or more doses of an effective amount of a composition comprising a PTPRS de-clustering agent and a TNF inhibitor or IL-6 inhibitor. Optionally, the kit comprises a non-enzymatic recombinant protein comprising an amino acid sequence of an extracellular domain of PTPRS or a subsequence, portion, homologue, variant or derivative thereof. Optionally, the kit comprises one or more portions of the extracellular domain of PTPRS. Optionally, the composition or protein is present in a container (e.g., vial or packet). Optionally, the kit comprises one or more additional agents for treating or preventing one or more symptom of an inflammatory and/or autoimmune disease. Optionally, the kit comprises a means of administering the composition, such as, for example, a syringe, needle, tubing, catheter, patch, and the like. The kit may also comprise formulations and/or materials requiring sterilization and/or dilution prior to use.

The compositions and agents as described herein are useful for both prophylactic and therapeutic treatment. For prophylactic use, a therapeutically effective amount of the agents described herein are administered to a subject prior to or during early onset (e.g., upon initial signs and symptoms of an autoimmune disease). Therapeutic treatment involves administering to a subject a therapeutically effective amount of the agents described herein after diagnosis or development of disease.

The provided proteins, agents and compositions are for use in the treatment of a subject who has or is at risk of developing an autoimmune disease, including for example arthritis such as rheumatoid arthritis. Optionally, the proteins, agents, and compositions are for use in the treatment of a subject who has or is at risk of developing an extracellular matrix disease and/or a fibroblast-mediated disease. As used herein, the term “extracellular matrix disease” refers to a condition, disorder or disease, associated with the extracellular matrix (ECM) or one or more components of the extracellular matrix. The extracellular matrix provides structural support to cells in addition to being involved in other biological functions including, but not limited to, intracellular communication. Components of the extracellular matrix include, but are not limited to, proteoglycans (e.g., heparan sulfate, chondroitin sulfate, and keratin sulfate), non-proteoglycan polysaccharides (e.g., hyaluronic acid), fibers, collagen, elastin, fibronectin and laminin. The extracellular matrix also serves as a depot for signaling molecules such as growth factors and cytokines. Extracellular matrix diseases include diseases associated with the dysregulation of one or more functions of the ECM (e.g., dysregulated intracellular communication and/or movement) or dysregulation of one or more components of the ECM (e.g., increased or decreased activity and/or production of one or more components of the ECM). Extracellular matrix diseases also include diseases associated with altered degradation and remodeling of the ECM and diseases associated with altered (e.g., increased or decreased) accumulation of agents, e g., immunocomplexes and other immune products, in the ECM. Extracellular matrix diseases include, but are not limited to, atherosclerosis, cancer, amyloid diseases, glomerular diseases, mesangial diseases, inflammatory conditions, and developmental disorders. As used herein, the term “fibroblast-mediated disease” refers to a condition, disorder, or disease, associated with fibroblast cell activity or movement. Fibroblasts are a type of cell involved in the synthesis of the ECM and collagen and are the major cell type of connective tissue. Types of fibroblasts include, but are not limited to, synovial fibroblasts, dermal fibroblasts, and interstitial fibroblasts. The main function of fibroblasts is to maintain the integrity of connective tissue by continuously secreting components of the ECM. Fibroblast-mediated diseases include diseases associated with the altered activity and/or movement of fibroblasts. Thus, for example, a fibroblast-mediated disease includes diseases associated with altered fibroblast migration or altered fibroblast activity. Fibroblast activities include, but are not limited to, collagen production, glycosaminoglycan production, reticular and elastic fiber production, cytokine production, and glycoprotein production. Thus, fibroblast-mediated diseases include diseases associated with altered production by fibroblasts of one or more of collagen, glycosaminoglycans, reticular and elastic fibers, cytokines, and glycoproteins.

As used herein, the term “inflammatory disease” refers to a disease or condition characterized by aberrant inflammation (e.g. an increased level of inflammation compared to a control such as a healthy person not suffering from a disease). Examples of inflammatory diseases include autoimmune diseases, arthritis, rheumatoid arthritis, psoriatic arthritis, juvenile idiopathic arthritis, multiple sclerosis, systemic lupus erythematosus (SLE), myasthenia gravis, juvenile onset diabetes, diabetes mellitus type 1, Guillain-Barre syndrome, Hashimoto's encephalitis, Hashimoto's thyroiditis, ankylosing spondylitis, psoriasis, Sjogren's syndrome, vasculitis, glomerulonephritis, auto-immune thyroiditis, Behcet's disease, Crohn's disease, ulcerative colitis, bullous pemphigoid, sarcoidosis, ichthyosis, Graves ophthalmopathy, inflammatory bowel disease, Addison's disease, Vitiligo, asthma, allergic asthma, acne vulgaris, celiac disease, chronic prostatitis, inflammatory bowel disease, pelvic inflammatory disease, reperfusion injury, ischemia reperfusion injury, stroke, sarcoidosis, transplant rejection, interstitial cystitis, atherosclerosis, scleroderma, and atopic dermatitis.

Provided herein are methods of modulating PTPRS activity in a subject, the method comprising administering to the subject an effective amount of a PTPRS de-clustering agent, wherein administration modulates PTPRS activity in the subject. Also provided are methods of treating, preventing, and/or ameliorating an autoimmune disease or disorder in a subject in need thereof. Specifically, provided is a method of treating an autoimmune disease in a subject, the method comprising administering to the subject a therapeutically effective amount of a PTPRS de-clustering agent and a TNF inhibitor or IL-6 inhibitor as described above, wherein administration treats the autoimmune disease in the subject. Also provided is a method of treating an autoimmune disease in a subject, the method comprising administering to the subject a therapeutically effective amount of a compound that increases PTPRS phosphatase activity, wherein administration treats the autoimmune disease in the subject. Autoimmune diseases or disorders include, but are not limited to, inflammatory autoimmune diseases. Optionally, the autoimmune disease is arthritis, rheumatoid arthritis, psoriatic arthritis, juvenile idiopathic arthritis, scleroderma, systemic scleroderma, multiple sclerosis, systemic lupus erythematosus (SLE), myasthenia gravis, juvenile onset diabetes, diabetes mellitus type 1, Guillain-Barre syndrome, Hashimoto's encephalitis, Hashimoto's thyroiditis, ankylosing spondylitis, psoriasis, Sjogren's syndrome, vasculitis, glomerulonephritis, auto-immune thyroiditis, Behcet's disease, Crohn's disease, ulcerative colitis, bullous pemphigoid, sarcoidosis, psoriasis, ichthyosis, Graves ophthalmopathy, inflammatory bowel disease, Addison's disease, Vitiligo, asthma, or allergic asthma. Optionally, the autoimmune disease is arthritis, Crohn's disease, scleroderma, or rheumatoid arthritis. Optionally, the compound is a PTPRS de-clustering agent and a TNF inhibitor or IL-6 inhibitor as described above. Optionally, the PTPRS de-clustering agent is not chondroitin sulfate. Optionally, the de-clustering agent is not chondroitin sulfate, a chondroitin sulfate mimetic or an agent that has the same or similar mechanism of action as chondroitin sulfate. Optionally, the PTPRS de-clustering agent is an anti-PTPRS antibody or fragment thereof, an anti-heparan sulfate antibody, or a chondroitin sulfate mimetic. Optionally, as described throughout, the PTPRS de-clustering agent can be a non-enzymatic recombinant protein comprising an amino acid sequence of an extracellular domain of PTPRS or a subsequence, portion, homologue variant or derivative thereof.

The methods include administering an effective amount of the provided agents and compositions, wherein administering the effective amount of the composition treats or prevents the autoimmune disease in the subject. Administration of a composition disclosed herein can be a systemic or localized administration. For example, treating a subject having an inflammatory autoimmune disorder can include administering an oral or injectable form of the pharmaceutical composition on a daily basis or otherwise regular schedule. Optionally, the agents and composition are formulated for administration can be formulated for delivery to synovial fluid and/or for delivery to fibroblast-like synoviocytes. In some embodiments, the treatment is only on an as-needed basis, e.g., upon appearance of inflammatory autoimmune disease symptoms.

Also provided are methods of decreasing fibroblast activity in a subject. The methods include administering to the subject a therapeutically effective amount of a PTPRS de-clustering agent, wherein administration decreases fibroblast activity in the subject. Optionally, the de-clustering agent is not chondroitin sulfate, a chondroitin sulfate mimetic or an agent that has the same or similar mechanism of action as chondroitin sulfate. In some embodiments, the de-clustering agent is a chondroitin sulfate mimetic. Optionally, the PTPRS de-clustering agent is a non-enzymatic recombinant protein as provided herein. Optionally, the PTPRS de-clustering agent binds heparan sulfate. Optionally, the PTPRS de-clustering agent is an anti-PTPRS antibody or fragment thereof or an anti-heparan sulfate antibody or fragment thereof. Optionally, the fibroblast activity comprises fibroblast migration. Optionally, the fibroblast activity comprises collagen production, glycosaminoglycan production, reticular and elastic fiber production, cytokine production, chemokine production, glycoprotein production or combinations thereof. Optionally, the fibroblast activity comprises extracellular matrix production. Fibroblasts include, but are not limited to, synovial fibroblasts, dermal fibroblasts, and interstitial fibroblasts. Optionally, the fibroblasts are synovial fibroblasts.

In some cases, the subject has a fibroblast-mediated disease. Thus, provided are methods of treating a fibroblast mediated disease in a subject. The methods include administering to the subject a therapeutically effective amount of a PTPRS de-clustering agent, wherein administration treats the fibroblast-mediated disease in the subject. Optionally, the de-clustering agent is not chondroitin sulfate, a chondroitin sulfate mimetic or an agent that has the same or similar mechanism of action as chondroitin sulfate. In some embodiments, the de-clustering agent is a chondroitin mimetic. Optionally, the PTPRS de-clustering agent is a non-enzymatic recombinant protein as provided herein. Optionally, the PTPRS de-clustering agent binds heparan sulfate. Optionally, the PTPRS de-clustering agent is an anti-PTPRS antibody or fragment thereof or an anti-heparan sulfate antibody or fragment thereof. Fibroblast-mediated diseases include, but are not limited to, fibrosis and fibroblast-mediated autoimmune diseases. The fibrosis can be, for example, pulmonary fibrosis, idiopathic pulmonary fibrosis, liver fibrosis, endomyocardial fibrosis, atrial fibrosis, mediastinal fibrosis, myelofibrosis, retroperitoneal fibrosis, nephrogenic systemic fibrosis, skin fibrosis, or arthrofibrosis. The fibroblast-mediated autoimmune disease can be, for example, Crohn's disease, arthritis, rheumatoid arthritis, and scleroderma.

Provided herein are methods of modulating extracellular matrix in a subject, the method comprising administering to the subject an effective amount of the non-enzymatic recombinant protein provided herein, wherein administration modulates the extracellular matrix in the subject. Optionally, the method does not include administration of chondroitin sulfate, a chondroitin sulfate mimetic or an agent that has the same or similar mechanism of action as chondroitin sulfate. Modulation of the extracellular matrix includes, for example, modulation of one or more components of the extracellular matrix. Optionally, the extracellular matrix component is selected from the group consisting of a proteoglycan, polysaccharide or fiber. Optionally, the extracellular matrix component is a proteoglycan, e.g., heparan sulfate or chondroitin sulfate. Optionally, the extracellular matrix component is heparan sulfate. Optionally, the subject has an extracellular matrix disease. Extracellular matrix diseases are known and include, but are not limited to, atherosclerosis, cancer, an amyloid disease, an inflammatory condition, and a developmental disorder. Optionally, the extracellular matrix disease is osteoarthritis. Optionally, the amyloid disease is Alzheimer's disease or inflammation-related AA amyloidosis. Optionally, the inflammatory condition is systemic sclerosis or lupus.

The herein provided methods that include the treatment of subjects with an inflammatory condition, autoimmune disease, fibroblast-mediated disease, or extracellular matrix disease can include administration of one or more additional agents that treat or prevent the inflammatory condition or autoimmune disease. For example, the provided methods can further include administration of and effective amount of one or more of anti-inflammatory agents. Suitable additional agents for use in the provided methods include, but are not limited to, analgesics, non-steroidal anti-inflammatory drugs, disease-modifying anti-rheumatic drugs, corticosteroids, and vitamin D analogues. Exemplary disease-modifying anti-rheumatic drugs for treating or preventing rheumatoid arthritis include, but are not limited to, azathioprine, cyclosporine A, D-penicillamine, gold salts, hydroxychloroquine, leflunomide, methotrexate (MTX), minocycline, sulfasalazine (SSZ), and cyclophosphamide.

Combinations of agents or compositions can be administered either concomitantly (e.g., as a mixture), separately but simultaneously (e.g., via separate intravenous lines) or sequentially (e.g., one agent is administered first followed by administration of the second agent). Thus, the term combination is used to refer to concomitant, simultaneous or sequential administration of two or more agents or compositions. The course of treatment is best determined on an individual basis depending on the particular characteristics of the subject and the type of treatment selected. The treatment, such as those disclosed herein, can be administered to the subject on a daily, twice daily, bi-weekly, monthly or any applicable basis that is therapeutically effective. The treatment can be administered alone or in combination with any other treatment disclosed herein or known in the art. The additional treatment can be administered simultaneously with the first treatment, at a different time, or on an entirely different therapeutic schedule (e.g., the first treatment can be daily, while the additional treatment is weekly).

According to the methods provided herein, the subject is administered an effective amount of one or more of the agents provided herein. The terms effective amount and effective dosage are used interchangeably. The term effective amount is defined as any amount necessary to produce a desired physiologic response (e.g., reduction of inflammation). Effective amounts and schedules for administering the agent may be determined empirically by one skilled in the art. The dosage ranges for administration are those large enough to produce the desired effect in which one or more symptoms of the disease or disorder are affected (e.g., reduced or delayed). The dosage should not be so large as to cause substantial adverse side effects, such as unwanted cross-reactions, anaphylactic reactions, and the like. Generally, the dosage will vary with the age, condition, sex, type of disease, the extent of the disease or disorder, route of administration, or whether other drugs are included in the regimen, and can be determined by one of skill in the art. The dosage can be adjusted by the individual physician in the event of any contraindications. Dosages can vary and can be administered in one or more dose administrations daily, for one or several days. Guidance can be found in the literature for appropriate dosages for given classes of pharmaceutical products. For example, for the given parameter, an effective amount will show an increase or decrease of at least 5%, 10%, 15%, 20%, 25%, 40%, 50%, 60%, 75%, 80%, 90%, or at least 100%. Efficacy can also be expressed as “-fold” increase or decrease. For example, a therapeutically effective amount can have at least a 1.2-fold, 1.5-fold, 2-fold, 5-fold, or more effect over a control. The exact dose and formulation will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Remington: The Science and Practice of Pharmacy, 20th Edition, Gennaro, Editor (2003), and Pickar, Dosage Calculations (1999)).

Optionally, the provided methods of treatment or method of modulating PTPRS activity or function in a subject further includes obtaining a biological sample from the subject and determining whether the subject has an altered RNA level or an altered protein level of PTPRS as compared to a control, an altered RNA level or altered protein level indicating the subject has or is at risk of developing an inflammatory condition, autoimmune disease, fibroblast-mediated disease or extracellular matrix disease. Optionally, the altered level is an elevated level as compared to a control. A control sample or value refers to a sample that serves as a reference, usually a known reference, for comparison to a test sample. For example, a test sample can be taken from a patient suspected of having autoimmune disease and compared to samples from a subject known to have an autoimmune disease or a known normal (non-disease) subject. A control can also represent an average value gathered from a population of similar individuals, e.g., autoimmune disease patients or healthy individuals with a similar medical background, same age, weight, etc. A control value can also be obtained from the same individual, e.g., from an earlier-obtained sample, prior to disease, or prior to treatment.

Thus, also provided are methods of determining whether a subject has or is at risk for developing an inflammatory condition, autoimmune disease, fibroblast-mediated disease or extracellular matrix disease comprising obtaining a biological sample from the subject and determining whether the subject has an elevated RNA level or an elevated protein level of PTPRS or isoform thereof, an elevated RNA level or elevated protein level indicating the subject has or is at risk of developing an autoimmune disease, inflammatory disease, fibroblast-mediated disease or extracellular matrix disease. Optionally, the provided methods further comprise selecting a subject with an autoimmune disease. Optionally, the autoimmune disease is an inflammatory autoimmune disease, e.g., arthritis or rheumatoid arthritis. As used herein, biological samples include, but are not limited to, cells, tissues and bodily fluids. Bodily fluids that used to evaluate the presence, absence or level of PTPRS RNA or protein include without limitation whole blood, plasma, urine, serum, tears, lymph, bile, cerebrospinal fluid, interstitial fluid, aqueous or vitreous humor, colostrum, sputum, amniotic fluid, saliva, a bronchioalveolar lavage sample, perspiration, transudate, exudate, and synovial fluid. Optionally, the biological sample is derived from a joint tissue or bodily fluid. Optionally, the provided methods further comprise isolating cells from the joint tissue or bodily fluid thereby forming an isolated cell sample. Such isolated cell samples can comprise synoviocytes, fibroblasts, hematopoetic cells, macrophages, leukocytes, T-cells or a combination thereof. Optionally, the synoviocytes are fibroblast-like synoviocytes or macrophage-like synoviocytes. Optionally, the isolated cell sample comprises fibroblast-like synoviocytes.

Methods for detecting and identifying nucleic acids and proteins and interactions between such molecules involve conventional molecular biology, microbiology, and recombinant DNA techniques within the skill of the art. Such techniques are explained fully in the literature (see, e.g., Sambrook, Fritsch & Maniatis, Molecular Cloning: A Laboratory Manual, Second Edition 1989, Cold Spring Harbor Laboratory Press, Cold Spring Harbor, N.Y.; Animal Cell Culture, R. I. Freshney, ed., 1986).

Methods for detecting RNA are largely cumulative with the nucleic acid detection assays and include, for example, Northern blots, RT-PCR, arrays including microarrays and sequencing including high-throughput sequencing methods. In some embodiments, a reverse transcriptase reaction is carried out and the targeted sequence is then amplified using standard PCR. Quantitative PCR (qPCR) or real time PCR (RT-PCR) is useful for determining relative expression levels, when compared to a control. Quantitative PCR techniques and platforms are known in the art, and commercially available (see, e.g., the qPCR Symposium website, available at qpersymposium.com). Nucleic acid arrays are also useful for detecting nucleic acid expression. Customizable arrays are available from, e.g., Affymetrix. Optionally, methods for detecting RNA include sequencing methods. RNA sequencing are known and can be performed with a variety of platforms including, but not limited to, platforms provided by Illumina, Inc., (La Jolla, Calif.) or Life Technologies (Carlsbad, Calif.). See, e.g., Wang, et al., Nat Rev Genet. 10(1):57-63 (2009); and Martin, Nat Rev Genet. 12(10):671-82 (2011).

Protein levels or concentration can be determined by methods standard in the art for quantitating proteins, such as Western blotting, ELISA, ELISPOT, immunoprecipitation, immunofluorescence (e.g., FACS), immunohistochemistry, immunocytochemistry, etc., as well as any other method now known or later developed for quantitating protein in or produced by a cell.

Disclosed are materials, compositions, and components that can be used for, can be used in conjunction with, can be used in preparation for, or are products of the disclosed methods and compositions. These and other materials are disclosed herein, and it is understood that when combinations, subsets, interactions, groups, etc. of these materials are disclosed that while specific reference of each various individual and collective combinations and permutations of these compounds may not be explicitly disclosed, each is specifically contemplated and described herein. For example, if a method is disclosed and discussed and a number of modifications that can be made to a number of molecules including the method are discussed, each and every combination and permutation of the method, and the modifications that are possible are specifically contemplated unless specifically indicated to the contrary. Likewise, any subset or combination of these is also specifically contemplated and disclosed. This concept applies to all aspects of this disclosure including, but not limited to, steps in methods using the disclosed compositions. Thus, if there are a variety of additional steps that can be performed, it is understood that each of these additional steps can be performed with any specific method steps or combination of method steps of the disclosed methods, and that each such combination or subset of combinations is specifically contemplated and should be considered disclosed.

Publications cited herein and the material for which they are cited are hereby specifically incorporated by reference in their entireties.

A number of embodiments have been described. Nevertheless, it will be understood that various modifications may be made. Accordingly, other embodiments are within the scope of the claims.

### EXAMPLES

### Example 1. Materials and Methods

Polypeptides

The following polypeptides were used in experiments:

″In use″ (SEQ ID NO: 16):
EEPPRFIREPKDQIGVSGGVASFVCQATGDPKPRVTWNKKGKKVNSQRF
ETIDFDESSGAVLRIQPLRTPRDENVYECVAQNSVGEITIHAKLTVLRE
DQLPPGFPNIDMGPQLKVVERTRTATMLCAASGNPDPEITWFKDFLPVD
PSASNGRIKQLRSGALQIESSEETDQGKYECVATNSAGVRYSSPANLYV
RTSGGGSLVPRGSEPKSCDKTHTCPPCPAPELLGGPSVFLFPPKPKDTL
MISRTPEVTCVVVDVSHEDPEVKFNWYVDGVEVHNAKTKPREEQYNSTY
RVVSVLTVLHQDWLNGKEYKCKVSNKALPAPIEKTISKAKGQPREPQVY
TLPPSRDELTKNQVSLTCLVKGFYPSDIAVEWESNGQPENNYKTTPPVL
DSDGSFFLYSKLTVDKSRWQQGNVFSCSVMHEALHNHYTQKSLSLSPGK
″Construct 1″ (SEQ ID NO: 17):
ETGEEPPRFIREPKDQIGVSGGVASFVCQATGDPKPRVTWNKKGKKVNS
QRFETIDFDESSGAVLRIQPLRTPRDENVYECVAQNSVGEITIHAKLTV
LREDQLPPGFPNIDMGPQLKVVERTRTATMLCAASGNPDPEITWFKDFL
PVDPSASNGRIKQLRSGALQIESSEETDQGKYECVATNSAGVRYSSPAN
LYVRTSGGGGSGGGGSEPKSCDKTHTCPPCPAPELLGGPSVFLFPPKPK
DTLMISRTPEVTCVVVDVSHEDPEVKFNWYVDGVEVHNAKTKPREEQYN
STYRVVSVLTVLHQDWLNGKEYKCKVSNKALPAPIEKTISKAKGQPREP
QVYTLPPSRDELTKNQVSLTCLVKGFYPSDIAVEWESNGQPENNYKTTP
PVLDSDGSFFLYSKLTVDKSRWQQGNVFSCSVMHEALHNHYTQKSLSLS
PGK
″His-tagged″ (SEQ ID NO: 18):
ETGEEPPRFIREPKDQIGVSGGVASFVCQATGDPKPRVTWNKKGKKVNS
QRFETIDFDESSGAVLRIQPLRTPRDENVYECVAQNSVGEITIHAKLTV
LREDQLPPGFPNIDMGPQLKVVERTRTATMLCAASGNPDPEITWFKDFL
PVDPSASNGRIKQLRSGALQIESSEETDQGKYECVATNSAGVRYSSPAN
LYVRGTKHHHHHH

Preparation of Human Synovial Tissue and FLS

FLS were obtained from the University of California, San Diego (UCSD) Clinical and Translational Research Institute (CTRI) Biorepository and from Showa University Division of Rheumatology. Each line was previously obtained from discarded synovial tissue from different RA patients undergoing synovectomy, as described in(52). The diagnosis of RA conformed to the American College of Rheumatology 1987 revised criteria(53). FLS were collected and used for experiments as approved by the UCSD Institutional Review Board (IRB) under protocol #140175, or used for experiments as approved by the La Jolla Institute for Allergy and Immunology IRB under protocol #CB-120-0614. All patients signed a consent form approved by the local IRB.

FLS were cultured in Dulbecco's modified Eagle's medium (DMEM, Corning) with 10% fetal bovine serum (FBS; Omega Scientific), 2 mM L-glutamine, 50 μg/ml gentamicin, 100 units/ml penicillin, and 100 μg/ml streptomycin (Life Technologies) at 37° C. in a humidified atmosphere containing 5% CO2. For all experiments, FLS were used between passages 4 and 10, and the cells were synchronized in 0.1% FBS (serum-starvation medium) for 24-48 h prior to experiments unless otherwise noted.

Human Dermal Fibroblast

Dermal fibroblasts lines from healthy human donors (NHDF) were isolated from skin specimens obtained from the National Disease Research Interchange (NDRI) as described(54). NHDF were cultured in the same complete DMEM medium and conditions as FLS. For all experiments, NHDF were used between passages 3 and 8, and cells were synchronized in serum-starved media with 0.1% FBS for 24 h prior to analysis or functional assays

Preparation of Mouse FLS Lines

Elbow, knee and ankle joints from 8-week old BALB/c mice were isolated. Minced tissues were digested in 0.5 mg/ml collagenase IV in RPMI-1640 for 2 h at 37° C. with gentle agitation and cultured for 4 days in FLS media (DMEM) containing 10% fetal bovine serum, 2 mM L-glutamine, 50 μg/ml gentamicin, 100 units/ml penicillin and 100 μg/ml streptomycin at 37° C. in a humidified 5% CO2 atmosphere. Murine FLS were used between passages 4 and 10, and synchronized overnight in 0.1% FBS (serum-starvation media) prior to experiments unless otherwise noted.

FLS Migration Assays

Confluent FLS were serum starved for 24h in DMEM containing 0.1% FBS, harvested by trypsin digestion and seeded at 5×10⁴ cells in 100 μl serum-free DMEM containing 0.5% BSA in the upper chamber of a 6.5 mm-diameter Transwell polycarbonate culture insert with a pore size of 8 μm (Costar). Inserts were placed in 24-well plates with 600 μl DMEM containing 10% FBS. The assay plates were incubated in presence/absence of Fc-Ig1&2 for 24h at 37° C. and 5% CO2, after which the Transwell inserts were removed and the upper chamber gently wiped with a cotton swab to remove non-migrating cells. Transwell membranes were fixed for 5 min in methanol and stained for 30 min in 0.2% crystal violet in 2% ethanol. Cells were visualized using a Motic AE2000 microscope at 10×. Cells were quantified by counting 4 non-overlapping fields using ImageJ software (NIH, version 1.8.0_201). For migration in the presence of TNF, RA FLS were serum starved for 24, then pre-treated with TNF (50 ng/mL) for an additional 24 h after which cells were seeded as for the migration assay. TNF (50 ng/mL) was also added in combination with Fc-Ig1&2 during the migration assay.

FLS Invasion Assay

For invasion assays, cells were seeded as for migration assays, except Corning™ BioCoat Matrigel Invasion Transwell chambers (Corning) were used. The assay plates were incubated for 48h before staining and imaging as described for migration assays.

Wound Healing Assay

RA FLS were grown to confluence in 6-well plates and serum starved for 24 h in DMEM with 0.1% FBS. Cells were scratched with a 1 ml tip and incubated in DMEM containing 1% FBS in the presence of 20 or 40 nM Fc-Ig1&2 or vehicle control. 4 Images of the wound was captured at the right after wounding (0h) and after 24h using a Motic AE2000 microscope at 4× with the software ToupView 3.7. Wound area was calculated using the ImageJ (NIH, version 1.8.0_201) software and the wound area after 24h was normalized to the area at 0h.

Immunoblotting

Cells were lysed in THE buffer (50 mM Tris-HCl [pH 7.5], 150 mM NaCl, 5 mM EDTA [pH 8.0]) containing 1 mM phenylmethanesulfonyl fluoride, 1× protease inhibitor cocktail (Roche) and PhosStop (Sigma-Aldrich). Protein concentration of cell lysates was determined using the Pierce BCA Protein Assay Kit (Thermo Scientific) Immunoblotting was performed using a goat polyclonal anti-human PTPRS antibody (R&D Systems) and the rabbit anti-GAPDH antibody from Cell Signaling.

For detection of Syndecan-4, RA and OA FLS lines were treated with a combination of Heparanase I (60 ng/mL), II (88 ng/mL) and III (90 ng/mL) for 1 h at 37 C after which cells were washed with PBS and lysed in 1×RIPA lysis buffer (Cell Signaling Technologies). Recombinant P. heparanus Heparanase II and III and recombinant F. heparanum Heparanase I was obtained from R&D systems. Syndecan-4 was detected using a goat polyclonal anti-Syndecan-4 antibody (R&D systems).

Quantitative Real-Time RT-PCR (qPCR)

RNA was extracted using the RNeasy kit (Qiagen). cDNA was synthesized using the SuperScript® III First-Strand Synthesis SuperMix for qRT-PCR (Life Technologies). qPCR was performed on a Bio-Rad CFX384 Real-Time PCR Detection System, with primer assays and SYBR® Green qPCR Mastermix from SABiosciences/Qiagen. Primer assay efficiencies were guaranteed by the manufacturer to be greater than 90%. Each reaction was measured using technical triplicates and data was normalized to the expression levels of the house-keeping gene, glyceraldehyde 3-phosphate dehydrogenase (GAPDH). Results are presented as fold change compared to either the expression level in control samples with the ΔΔCq method or as fold change compared to the expression of GAPDH.

Mice

All animal experiments were carried out in accordance with the Institutional Animal Care and Use Committee-approved protocol (#AP140-NB4-0610) at the La Jolla Institute for Allergy & Immunology and UCSD (#S16098). PTPRS knockout (KO) mice on a BALB/c background were generated as previously described in(55). DBA1/J (JAX 000670, DBA1/J), BALB/c (JAX 000651, BALB/cJ) and CD45.1 BALB/c (JAX 006584, CByJ.SJL(B6)-Ptprca/J) were obtained from Jackson Laboratories. KRN mice on the C57BL/6 background were a generous gift from Dr. Christophe Benoist (Harvard University). NOD mice were obtained from Taconic.

Arthritis Models

KRN and NOD mice were crossed to obtain offspring that developed arthritis at around 6-7 weeks of age (spontaneous K/B×N mice). Serum from arthritic K/B×N mice was pooled for use in the K/B×N serum transfer induced arthritis (STIA) model(35). To elicit STIA, 6-8 week old mice were injected intra-peritoneally (i.p.) with 100 μl of arthritogenic K/B×N serum. Severity of arthritis was evaluated by clinical scoring (as described below) and measurement of ankles swelling every other day, starting on the day of serum injection.

The collagen-induced arthritis (CIA) model was performed as described in(56). Briefly, 8-10 week-old male DBA/1J were immunized with 100 μg chicken type II collagen (Condrex) emulsified in Freund's adjuvant containing 50 μg of Mycobacterium tuberculosis (H37Ra, ATCC 25177) (CFA, Sigma-Aldrich). After 28 days, mice were boosted with 100 μg chicken type II collagen emulsified in incomplete Freund's adjuvant (IFA; Sigma-Aldrich). Arthritis was assessed by clinical scoring as described below. In all models, arthritis was clinically scored in wrists and ankles as previously described(57): 0=normal; 1=minimal erythema and mild swelling; 2=moderate erythema and mild swelling; 3=marked erythema and severe swelling, digits not yet involved; 4=maximal erythema and swelling, digits involved.

In Vivo Administration of his-Ig1&2, Fc-Ig1&2 and mEtanercept

Preparation of recombinant PTPRS 6×HisIg1&2 (here called His-Ig1&2) was described in(21). Preparation of human IgG1-Fc-fused Ig1&2 (here called Fc-Ig1&2) was contracted to LakePharma (USA). Murine TNF-blocking biologic p75TNFR:Fc (mEtanercept) was obtained from Amgen through the Amgen Extramural Research Alliance Program. His-Ig1&2 in Tris-buffered saline (TBS) or vehicle control (TBS), Fc-Ig1&2 in 20 mM Tris with 120 mM NaCl or human IgG1-Fc control and mEtanercept was administered according to the schedule described in the Figure Legends.

In Vivo pDC Depletion

pDCs were depleted in mice by 2 administrations of 500 μg anti-PDCA-1 (InVivoMAb anti-mouse CD317; BioXcell) or IgG isotype control (BioXcell) by i.p. or retro-orbital (r.o.) injection 2 days apart.

Measurement of Serum Anti-Collagen Antibody Levels

Anti-collagen antibody levels in sera of mice immunized with collagen were measured by enzyme-linked immunosorbent assay (ELISA) as described(56). Briefly, low-binding 96 multi-well plates (Costar) were coated with type II chicken sternal collagen (1 μg/ml; Sigma). Sera were incubated in serial dilutions, and IgG binding to collagen was detected using a biotinylated anti-mouse IgG antibody (Jackson Laboratories) as well as anti-mouse IgG1, IgG2a, IgG2b and IgG3 antibodies (Southern Biotech) followed by incubation with extravidin-HRP (Sigma) and exposure with 3,3′,5,5′-tetramethylbenzidine (TMB) substrate. Plate absorbance was read at 450 nm using a Tecan Infinite M1000 plate-reader.

Histological Scoring of Mouse Arthritic Joints

Whole hind paws were fixed in 10% formalin, decalcified, trimmed and embedded. Sections were prepared from tissue blocks and stained with H&E, Safranin-O or Toluidine blue (HistoTox). Histopathological scoring was performed as described(57). Briefly, joints of arthritic mice were assigned scores of 0-4 for inflammation based on H&E staining, according to the following criteria: 0=normal; 1=minimal infiltration of inflammatory cells in periarticular area; 2=mild infiltration; 3=moderate infiltration; and 4=marked infiltration. Joints of arthritic mice were given scores of 0-4 for bone resorption based on H&E staining, according to the following criteria: 0=normal; 1=minimal (small areas of resorption, not readily apparent on low magnification); 2=mild (more numerous areas of resorption, not readily apparent on low magnification, in trabecular or cortical bone); 3=moderate (obvious resorption of trabecular and cortical bone, without full thickness defects in the cortex; loss of some trabeculae; lesions apparent on low magnification); and 4=marked (full-thickness defects in the cortical bone and marked trabecular bone loss). Cartilage depletion was identified by diminished Safranin 0 or Toluidine blue staining of the matrix and was scored on a scale of 0-4, where 0=no cartilage destruction (full staining with Safranin 0), 1=localized cartilage erosions, 2=more extended cartilage erosions, 3=severe cartilage erosions and 4=depletion of entire cartilage. Histologic analyses were performed in a blinded manner. Images of whole ankles were acquired using the Zeiss Axioscan.Z1 (Zeiss) slide scanner and analyzed using Zen software (Zeiss)

Micro-Computed Tomography (microCT)

Mouse ankles were placed in 10% neutral-buffered formalin. After fixation, samples were transferred to 70% ethanol. Before scanning, bones were transferred to phosphate-buffered saline (PBS) for 48 h. Scanning was performed on a Skyscan1176 micro-CT (Bruker) with a voxel size of 9 μm, at 50 kV/200 mA, with a 0.5 mm aluminum filter. Exposure time was 810 ms. The X-ray projections were obtained at 0.4° intervals with a scanning angular rotation of 180° and a combination of 4 average frames. The projection images were reconstructed into 3D images using NRECON software (Bruker) and Data Viewer (Bruker). Data was processed using CT Analyzer software (Bruker) and images were generated using CT-VOX software (Bruker). Bone erosion was quantified as described in(56).

Radio Labeling of Fc-Ig1&2 & mEtanercept

To allow for radiolabeling, the agents were first functionalized with DOTA to provide chelation sites to attach the radioactive atom. To covalently couple Fc-Ig1&2 to DOTA (Tetraxetan), typically 0.8 mg of protein were immobilized on 0.5 ml heparan-agarose beads (Sigma) in a total of 1.3 ml Tris pH 7.3, 150 mM NaCl. DOTA-NHS ester (Macrocyclics) was added at a concentration of 0.77 mM and incubated at room temperature for 30′. After extensive washing in the same buffer, the protein was eluted in 750 mM NaCl, 20 mM Tris pH 8.3, further purified by size exclusion chromatography (Bio-Rad SEC 650) and concentrated to 12-15 mg/ml. mEtanercept was modified in a similar way, except that no immobilization on solid support was necessary. The reaction was routinely monitored by Native PAGE.

Following this functionalization step, the conjugates are incubated with Indium-111 for 3 hours at 43° C., and purified through a P10 separation column.

Bio-Distribution

STIA was induced in by an IP injection of 100 uL of K/B×N serum as described under arthritis models. Injection of radio-labeled proteins and subsequent imaging began at the peak of arthritis, which occurs 8 days post serum injection.

All animals were anesthetized with isoflurane before injections and imaging. Each animal received an intravenous injection of radiolabeled agent, with a dose of approximately 170 uCi per animal. Four arthritic mice were injected with approximately 170 uCi of ¹¹¹In-labeled-Fc-Ig1&2 and compared against two forms of control: five more arthritic mice injected with 280uCi of ¹¹¹In-labeled-mEtanercept, and five non-arthritic mice injected with 305 uCi of ¹¹¹In-labeled-Ig1&2 Immediately post injection, each animal was loosely restrained on the surface of the γ-Imager planar gamma imaging system (Biospace Labs, Nesles la Vallee, France), with continued anesthesia. The animals were placed on their backs with paws loosely taped to the top of the imager and the ¹¹¹In collimator in place. Each animal was imaged for 10 minutes at 1 hour, 6 hours, 1 day, 3 days, and 5 days post injection. Each image also included a standard with 5% of the injected dose, placed as a point source next to the animal. Following the final image, the mice were sacrificed and samples were collected of blood, bladder, liver, kidney, spleen, heart, lung, brain, eye and paws into plastic scintillation vials, weighed, and the activity in each vial was measured in a Gamma-9000 gamma counter (Beckman Coulter, Brea, Calif.). Images were analyzed with GammaVision software, calculations were performed in Excel, and graphs were constructed in SigmaPlot. Regions of interest (ROI's) were drawn around the hind paws and the standard at each timepoint. Total counts and ROI area were recorded for each ROI and exported into Excel. There, all the counts were corrected for decay and the total counts for each ROI were divided by the ROI area to provide a value of signal/area for each paw and the standard. All the values for the standards were normalized to 5%. This produced a correction factor that allowed all paw values to be converted from raw counts into percent of injected dose. The percent injected dose of both hind paws at each time point was averaged together and the resulting values were then averaged across all mice at each time point and plotted. Gamma counter values for each organ were used to calculate the percent of injected dose per organ uptake at day 5. A value of 72 mL/kg of blood was used to calculate the total volume and thus total signal for blood in each animal, from a smaller quantity collected at sacrifice. These percent of the injected dose per organ values were also compared and plotted across each mouse group.

Flow Cytometry

Single cell suspensions were prepared from lymph nodes and spleen. For isolation of cells from mouse blood, blood was collected into 2 mM EDTA in PBS via retro-orbital bleed (from live animals) or cardiac puncture (from euthanized animals). Erythrocytes were lysed from spleen and mouse blood using eBiosience RBC lysis buffer (Thermo Fisher). For isolation of synovial cells, ankle-joints were collected and the tibia and digits disarticulated by pulling with blunt forceps and bone marrow flushed out to avoid bone marrow contamination. Joints were rinsed with PBS and dissociated with Liberase™ (Roche) and DNasel at 37° C. for 60 min at 37° C. Cells were pre-incubated with Fc block (BD Pharmingen) before antibody staining. For surface staining, fluorochrome-conjugated antibodies specific for CD3 (17A2), CD19 (6D5), NK1.1 (PK136), CD11b (M1/70), CD44 (IM7), Ly6G (1A8), Ly6C (HK1.4), CD45.1 (A20), MHCII (M5/114.15.2), CD64 (X54-5/7.1), CD115 (AFS98), were obtained from Biolegend. CD43 (R2/60), PD1 (RMP1-30), GL7 (GL-7), CD25 (PC61.5), B220 (RA3-6B2), CD317/BST2 (eBio927), CD62L (MEL-14), CD45.2 (104), TCR-beta (H57-597), CD8 (53-6.7), CD4 (RM4-5) were obtained from eBioscience/Thermo Fisher. For intracellular cytokine staining, cells were incubated with 20 ng/mL Phorbol 12-myristate 13-acetate (PMA, Sigma Aldrich) and 1 μm Ionomycin (Sigma Aldrich) in the presence of Brefeldin A (3 microg/mL, eBioscience/Thermo Fisher) for 5h at 37°. Intracellular staining was performed with the IC fixation buffer (eBioscience/Thermo Fisher) and permeabilization buffer (eBioscience/Thermo Fisher). For intracellular staining of transcription factors the FoxP3/Transcription Factor staining buffer set was used (eBioscience/Thermo Fisher). Antibodies recognizing FoxP3 (FJK-16s), IL-17A (eBio17B7) and IFNgamma (XMG1.2) were obtained from eBioscience/Thermo Fisher and BCL6 from BD Bioscience. Dead cells were excluded from analysis by staining with Fixable Viability dye from eBioscience/Thermo Fisher.

Data were acquired on a ZE5 flow cytometer (Bio-Rad) equipped with Everest Software. Analysis were performed using FlowJo software (TreeStar). Cell sorting was performed on a FACSAria III instrument (BD Biosciences). For flow analysis, anti-mouse PTPRS (MEDIMABS) was labeled with AlexaFluor 647 using Mix-n-Stain™ antibody labeling kit (Sigma-Aldrich).

Bone Marrow Reconstitution

To generate bone marrow chimeras, male BALB/cByJ (CD45.1 congenic) mice were lethally irradiated with 2 doses of 550 Rads using an RS 2000 Biological irradiator and subsequently administered bone-marrow from male PTPRS WT or KO congenic CD45.2 donor mice. KB×N serum was administered to induce arthritis 8 weeks post-irradiation. After 7 weeks, chimerism was verified by flow cytometry by staining for the appropriate CD45 allele (anti-CD45.1 and anti-CD45.2; eBioscience). The percentage of engraftment in recipient mice was greater than 95%.

M1 and M2 Polarization of Bone Marrow Derived Macrophages

Bone marrow cells were isolated from 8 to 12-week old BALB/c mice. Total bone marrow cells were plated in 6 well culture plates (2×10⁶ cells per well) in IMDM media containing 10% FBS, 25 mM HEPES, 2 mM L-Glutamin, 100 units/ml penicillin, 100 microg/ml streptomycin. Non adherent cells were removed after 24h by washing with PBS and replacing with new IMDM media containing 20 ng/mL M-CSF. Media was replaced every 3 days and cells were cultured for a total of 7 days. After 7 days BMDM were left unstimulated (M0) or stimulated with either 100 ng/mL LPS (Sigma) and 50 ng/mL IFNgamma (Biolegend) for M1 polarization or 10 ng/mL IL-4 (Biolagend) or 10 ng/mL IL-13 (Biolegend) for M2 polarization. Fc-Ig1&2 (20, 40 and 80 nM) Cells were collected after 24h and RNA extracted using the RNeasy Micro Kit (Qiagen). Expression of M1 related genes Tnf, Mb, Nos2, Il12b and M2 related genes Pparg, Arg1, Retnla and Mrc1 were analyzed by qPCR. Expression was normalized to the expression of Gapdh.

Phagocytosis Assay

Bone marrow cells were isolated from 8 to 12-week old BALB/c mice and cultured in 12 well plates (1×10⁶ cells per well) and cultured under BMDM conditions as described above. After 7 days BMDM were either left unstimulated (M0 condition) or under M1 or M2 polarizing conditions as described above. Fc-Ig1&2 (20, 40 or 80 nM) were added during M1 and M2 polarization. After 24h, polarizing media was replaced with IMDM without FBS and cells were cultured for 1h in the presence of either Fc-Ig1&2 (20, 40 or 80 nM) or vehicle control for 1h. After 1h either pHrodo™ Red E. Coli or pHrodo™ Green S. aureus BioParticles™ (Invitrogen) were added to the cells according to manufacturer's protocol. Cells were allowed to phagocytose bioparticles for 30 minutes, after which cells were washed, detached from plates using TrypLE Express (Thermo Fisher), stained with Fixable Viability Dye 780 (Thermo Fisher) and analyzed by flow cytometry. Cells incubated with Bioparticles on ice was used as negative control for phagocytosis. The pH sensitive dye pHrodo™ is non-fluorescent at neutral pH but fluoresce brightly upon the acidification that occurs in the phagosome.

CD4 T Cell Differentiation Assays.

Naïve CD4 T cells were isolated from pooled spleen and lymph nodes of either 8-12 week old BALB/c or DBA/1J mice using the EasySep™ Mouse Naïve CD4+ T cell isolation kit (Stem Cell Techonologies). Isolated naïve CD4 T cells were polarized into either Th1, Th17 or iTregs in the presence of Fc-Ig1&2 (20, 40 or 80 nM) or vehicle control using the below described conditions.

For differentiation of Th1 cells, 1×10⁵ naïve CD4 T cells were cultured on anti-CD3 (145-2C11, Biolegend, 2 microg/ml) coated plates in complete RPMI media (10% FBS, 100 units/mL penicillin, 100 microg/mL streptomycin, 1× nonessential amino acids, 25 mM HEPES, 55 microM beta-mercaptoethanol, 2 mM L-Glutamin) containing soluble anti-CD28 (37.51, Biolegend, 0.5 microg/mL), anti-IL4 (11B11, Biolegend, 10 μg/mL), recombinant mouse IL-12 (R&D Systems, 25 ng/mL) and recombinant mouse IL-2 (R&D Systems, 25 ng/mL). For differentiation of Th17 cells, 1×10⁵ naïve CD4 T cells were cultured on anti-CD3 (145-2C11, 2 microg/ml) coated plates in complete RPMI media containing soluble anti-CD28 (37.51, 1 μg/mL), anti-IFNgamma (XMG1.2, Biolegend, 10 microg/mL), anti-IL4 (11B11, 10 μg/mL), recombinant human TGFbeta1 (R&D Systems, 5 ng/mL) and recombinant mouse IL-6 (Biolegend, 50 ng/mL). For iTreg differentiation, 1×10⁵ naïve CD4 T cells were cultured on anti-CD3 (145-2C11, 2 microg/ml) coated plates in complete RPMI media containing soluble anti-CD28 (37.51, 0.5 microg/mL) and recombinant human TGFbeta1 (R&D Systems, 5 ng/mL). Cells were cultured under Th polarizing conditions for 5 days. To verify Th1 and Th17 differentiation cells were stimulated with PMA (20 ng/mL) and ionomycin (1 μM) in the presence of Brefeldin A (Thermo Fisher, 3 microg/mL) for 4h and analyzed by flow cytometry for the expression of IFNgamma and IL-17A. To verify iTreg differentiation, cells were analyzed by flow cytometry for the expression of FoxP3. Cells cultured without polarizing conditions (Th0) was used as control for polarization.

In a second experiment, we performed polarization of naïve CD4 T cells in the presence of antigen presenting cells (APCs). 1×10⁵Naïve CD4 T cells, isolated from BALB/c mice, were cultured with 1×10⁵ irradiated BALB/c Rag2-KO splenocytes (3,500 rad) as APCs and soluble anti-CD3 (145-2C11, Biolegend, 5 microg/ml) under Th1, Th17 or iTreg polarizing conditions. The same polarizing conditions as described above was used, however soluble anti-CD28 was removed due to the presence of irradiated APCs. Differentiation of Th1, Th17 and iTregs were verified as described above.

Transfection and Dual Luciferase Reporter Assay

Dual luficerase assays was performed in HEK293T cells obtained from ATCC. Confluent HEK293T cells were transfected using polyethylenimine (PEI) in a ratio of 3 to 1 per microgram plasmid in Opti-MEM. Overexpression vector for human USF2 or an empty control vector was obtained from VectorBuilder Inc. A luciferase promoter region reporter vector containing a 120 base pair portion of the PTPRS promoter region (UCSC Genome Browser on Human GRCh37/hg19 position: chr19:5,340,976-5,341,095) containing binding sites for USF2 was obtained from VectorBuilder Inc. Cells were incubated for 24 h after transfection. Luciferase activity was assessed using the Dual-Luciferase Reporter Assay System from Promega according to manufacturer's protocol. Renilla luciferase activity was used to normalize firefly luciferase activity.

Chromatin Immunoprecipitation

RA FLS were cultured to confluency, serum-starved for 24 and stimulated with recombinant human TNFalpha (50 ng/mL) for 6h or left unstimulated. Then cells were fixed in 1% formaldehyde for 15 min at RT. After sonication, chromatin was immunoprecipitated with the Pierce™ Magnetic ChIP assay Kit (Thermo Fisher) using a rabbit anti-USF2 antibody (NBP2-56717, Novus Biologicals) overnight at 4° C. according to manufacturer's instructions. The eluted DNA was used for PTPRS promoter region PCR and qPCR. 10% input for each condition was used for normalization. Human PTPRS promoter primers, 5′-TCTGCCCCGCTTCACATCG-3′ (forward) (SEQ ID NO: 19) and 5′-AGCCGCCACCACCACCACCA-3′ (reverse) (SEQ ID NO: 20), were purchased from IDT and used for ChIP qPCR with the PowerUp SYBR Green PCR master mix (Thermo Fisher) and PCR using the OneTaq Hot Start Quick-Load 2× Master Mix with GC buffer (New England Biolabs Inc)

siRNA Mediated Knock-Down

RA FLS were grown to confluency (70%) and transfected with 1 microg ON-TARGETplus SMARTpool siRNA targeting human USF2 (Dharmacon) or 1 microg of ON-TARGETplus Non-targeting Pool siRNA (Dharmacon) using the Human Dermal Fibroblast Nucleofector™ Kit (Lonza) according to manufacturer's protocol. In brief, 5×10⁵ RA FLS were resuspended in 100 microL of nucleofector solution containing siRNA and electoporated with program U-23 on an Amaxa Nucleofector II. Transfected cells were divided into 3 individual wells in a 6 well plate. Cells were harvested for RNA extraction after 24h and 48h and expression of USF2 and PTPRS was analyzed by qPCR and normalized towards the expression of GAPDH as described above. Fold change in expression was calculated against the expression in RA FLS treated with control siRNA using the ΔΔCT method.

Statistical Analysis

Sample sizes were selected based upon our experience with the assays being performed in order to achieve sufficient power to detect biologically relevant differences in the experiments being conducted. The two-way or one-way analysis of variance (ANOVA), Spearman correlation, unpaired t-test and Mann-Whitney U test were performed where appropriate as reported in the figure legends. All statistical analyses were performed using GraphPad Prism software. A comparison was considered significant if P was less than 0.05.

### Example 2

### Synoviocyte-Targeted Combination Therapy for Rheumatoid Arthritis

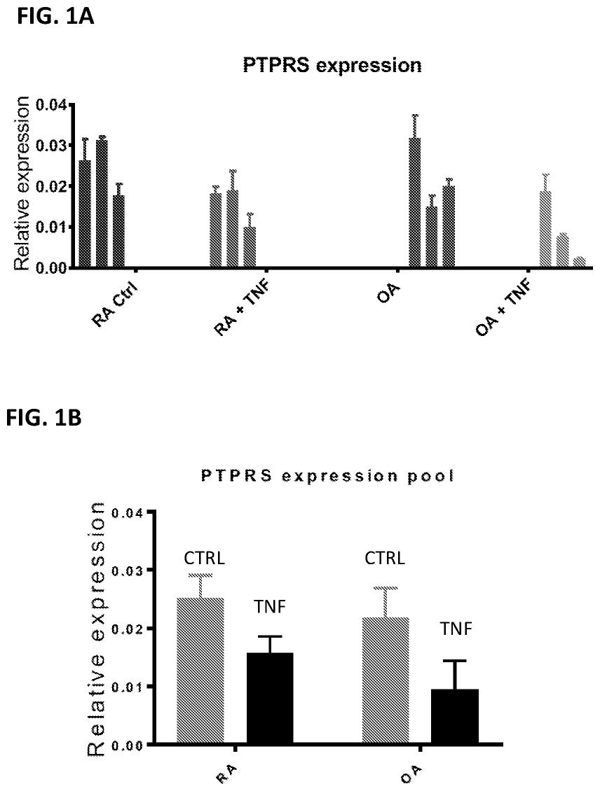

The effect of TNF on PTPRS (encoding RPTPσ) expression was studied in rheumatoid arthritis (RA) and osteoarthritis (OA) Fibroblast-Like Synoviocytes (FLS). FLS were used between passages 4 and 10, and the cells were synchronized in 0.1% FBS (serum-starvation medium) for 24-48 h prior to stimulation with 50 ng/ml TNF for 24h. RNA was extracted using the RNeasy kit (Qiagen). cDNA was synthesized using the SuperScript® III First-Strand Synthesis SuperMix for qRT-PCR (Life Technologies). qPCR was performed on a Bio-Rad CFX384 Real-Time PCR Detection System, with primer assays and SYBR® Green qPCR Mastermix from SABiosciences/Qiagen. Primer assay efficiencies were guaranteed by the manufacturer to be greater than 90%. Each reaction was measured using technical triplicates and data was normalized to the expression levels of the house-keeping gene, glyceraldehyde 3-phosphate dehydrogenase (GAPDH). Results are presented as fold change compared to either the expression level in control samples with the deltadeltaCq method or as fold change compared to the expression of GAPDH. For immunoblotting cells were lysed in THE buffer (50 mM Tris-HCl [pH 7.5], 150 mM NaCl, 5 mM EDTA [pH 8.0]) containing 1 mM phenylmethanesulfonyl fluoride, 1× protease inhibitor cocktail (Roche) and PhosStop (Sigma-Aldrich). Protein concentration of cell lysates was determined using the Pierce BCA Protein Assay Kit (Thermo Scientific). Immunoblotting was performed using a goat polyclonal anti-human PTPRS antibody (R&D Systems) and the rabbit anti-GAPDH antibody from Cell Signaling. FIG. 1A shows PTPRS expression in RA (n=3) and OA (n=3) FLS, with or without TNF. FIG. 1B shows PTPRS expression pool in RA and OA (n=3). Data were analyzed using two-way analysis of variance (ANOVA, n. P>0.05).

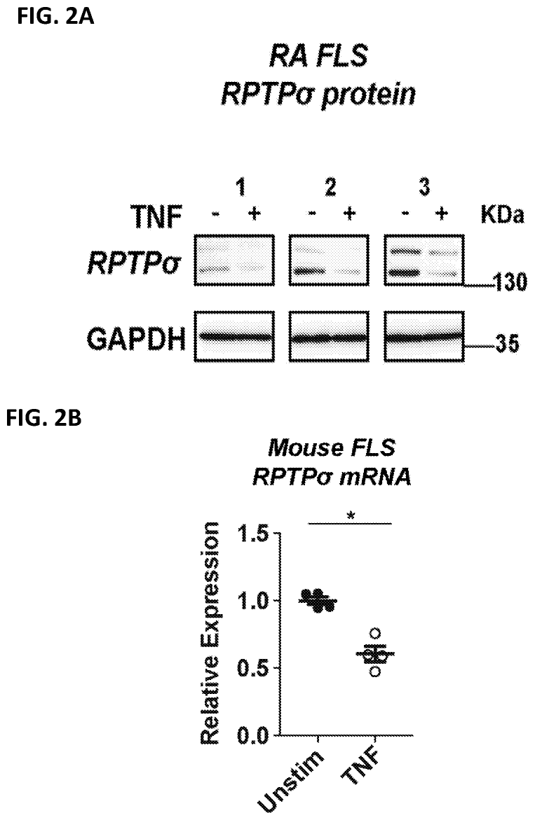

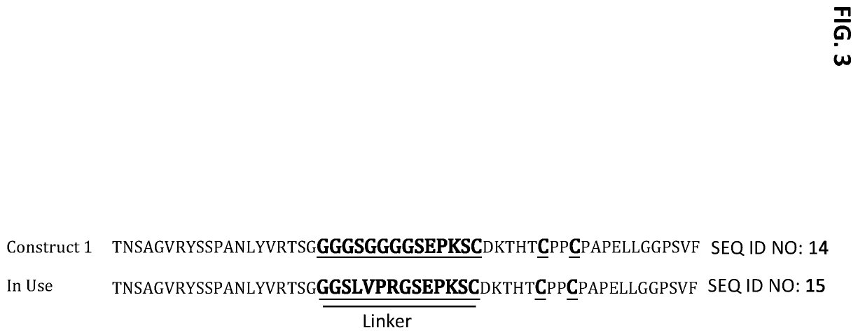

TNF-induced RPTPσ expression in mouse FLS was studied. Mouse FLS were serum-starved for 24 hours and then stimulated with 50 ng/ml of TNF or unstimulated for 24 hours. FIG. 2A shows RA FLS RPTPσ protein expression with or without TNF stimulation.

Elbow, knee and ankle joints from 8-week old BALB/c mice were isolated. Minced tissues were digested in 0.5 mg/ml collagenase IV in RPMI-1640 for 2 h at 37° C. with gentle agitation and cultured for 4 days in FLS media (DMEM) containing 10% fetal bovine serum, 2 mM L-glutamine, 50 μg/ml gentamicin, 100 units/ml penicillin and 100 μg/ml streptomycin at 37° C. in a humidified 5% CO2 atmosphere. Murine FLS were used between passages 4 and 10, and synchronized overnight in 0.1% FBS (serum-starvation media) prior to stimulation with 50 ng/ml TNF or unstimulated for 24h. RPTPσ mRNA expression levels (N=4) were measured by qPCR as described above. FIG. 2B shows the mouse FLS RPTPσmRNA relative expression, with or without TNF stimulation. RPTPσ mRNA expression levels (n=4) were measured by qPCR. The graph shows means+/− s.e.m. relative expression following normalization to the housekeeping gene POLR2A. Data were analyzed using the two-tailed Man-Whitney test (*, P<0.05). RPTPσ expression was reduced in the presence of TNF both in OA and RA FLS.

Linker sequences for Fc-Ig1&2 “in use” construct (TNSAGVRYSSPANLYVRTSGGGSLVPRGSEPKSCDKTHTCPPCPAPELLGGPSVF, SEQ ID NO: 15) and the Fc-Ig1&2 “Construct 1” construct (TNSAGVRYSSPANLYVRTSGGGGSGGGGSEPKSCDKTHTCPPCPAPELLGGPSVF, SEQ ID NO: 14). FIG. 3 shows the difference in amino acidic sequence between Fc-fusion constructs “in use” (initially generated by subcloning of the His-tagged construct utilized in the Doody K M, et al., “Targeting phosphatase-dependent proteoglycan switch for rheumatoid arthritis therapy. Sci Transl Med. 2015 May 20; 7(288):288ra76) and “construct 1” (utilized for most of the examples shown here). Construct “in use” had a protease cutting site in the linker (region between the Fc and the Ig1&2 portion of the construct) that was removed in the new construct 1. Underlined region indicated the linker and two cysteines that enable disulfide bonds within the Fc portion of the fusion protein (unchanged between the two constructs).

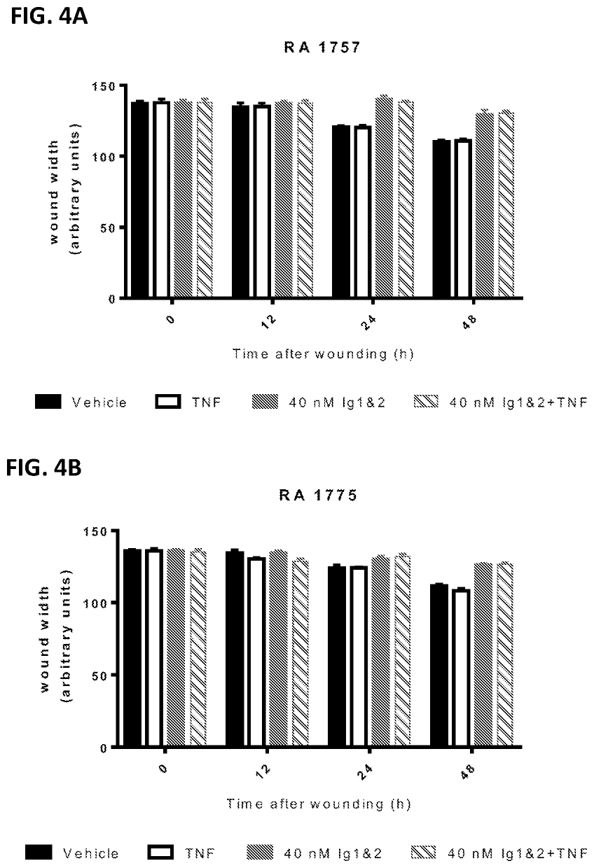

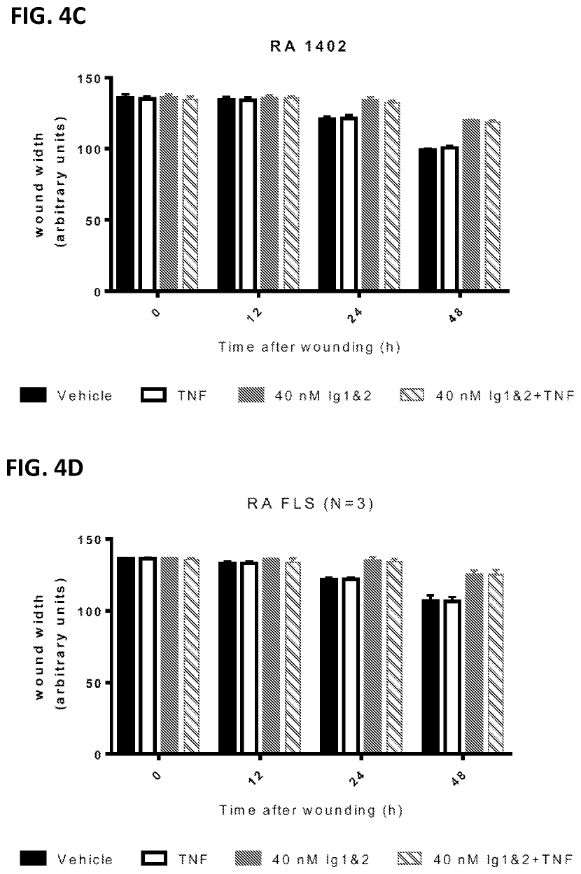

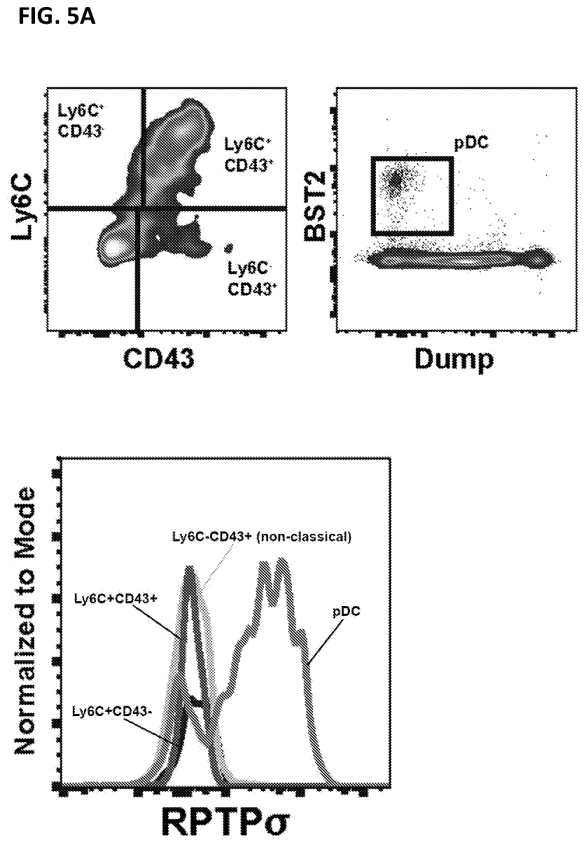

A scratch assay was performed with Fc-Ig1&2 Construct 1. RA FLS were grown to confluence in 6-well plates and serum starved for 24 h in DMEM with 0.1% FBS. Cells were scratched with a 1 ml tip and incubated in DMEM containing 1% FBS in the presence of 20 or 40 nM Fc-Ig1&2 or vehicle control. Images of the wound was captured at the right after wounding (0h) and after 24h using a Motic AE2000 microscope at 4× with the software ToupView 3.7. Wound area was calculated using the ImageJ (NIH, version 1.8.0_201) software and the wound area was measured at 0-12-24-48h. 50 ng/ml TNF, 40 nM PTPRS Ig1&2, was compared to 50 ng/ml TNF+40 nM PTPRS Ig1&2. FIG. 4A shows wound with (in arbitrary units) in RA 1757 at the time of wounding (0 hours), or at 12, 24, or 48 hours after wounding, in the presence or absence of TNF or Ig1&2, alone or in combination, as described above. FIG. 4B shows wound with (in arbitrary units) in RA 1775 at the time of wounding (0 hours), or at 12, 24, or 48 hours after wounding, in the presence or absence of TNF or Ig1&2, alone or in combination, as described above. FIG. 4C is a bar graph showing wound with (in arbitrary units) in RA 1402 at the time of wounding (0 hours), or at 12, 24, or 48 hours after wounding, in the presence or absence of TNF or Ig1&2, alone or in combination, as described above. FIG. 4D shows pooled data. Data were analyzed using two-way analysis of variance (ANOVA, ****, P<0.0001).

These results suggest that TNF-stimulated cells sufficient expression of PTPRS is retained to secure effectiveness of Ig1&2 in monotherapy

KRN and NOD mice were crossed to obtain offspring that developed arthritis at around 6-7 weeks of age (spontaneous K/B×N mice). Serum from arthritic K/B×N mice was pooled for use in the K/B×N serum transfer induced arthritis (STIA) model(35). To elicit STIA, 6-8 week old mice were injected intra-peritoneally (i.p.) with 100 μl of arthritogenic K/B×N serum. Severity of arthritis was evaluated by clinical scoring of wrists and ankles as previously described(57): 0=normal; 1=minimal erythema and mild swelling; 2=moderate erythema and mild swelling; 3=marked erythema and severe swelling, digits not yet involved; 4=maximal erythema and swelling, digits involved and measurement of ankles swelling every other day, starting on the day of serum injection.

For flow cytometry in order to isolate cells from mouse blood, blood was collected into 2 mM EDTA in PBS via retro-orbital bleed (from live animals) or cardiac puncture (from euthanized animals). Erythrocytes were lysed from spleen and mouse blood using eBiosience RBC lysis buffer (Thermo Fisher). For isolation of synovial cells, ankle-joints were collected and the tibia and digits disarticulated by pulling with blunt forceps and bone marrow flushed out to avoid bone marrow contamination. Joints were rinsed with PBS and dissociated with Liberase™ (Roche) and DNasel at 37° C. for 60 min at 37° C. Cells were pre-incubated with Fc block (BD Pharmingen) before antibody staining. For surface staining, fluorochrome-conjugated antibodies specific for CD3 (17A2), CD19 (6D5), NK1.1 (PK136), CD1 lb (M1/70), CD44 (IM7), Ly6G (1A8), Ly6C (HK1.4), CD45.1 (A20), MHCII (M5/114.15.2), CD64 (X54-5/7.1), CD115 (AFS98), were obtained from Biolegend. CD43 (R2/60), PD1 (RMP1-30), GL7 (GL-7), CD25 (PC61.5), B220 (RA3-6B2), CD317/BST2 (eBio927), CD62L (MEL-14), CD45.2 (104), TCR-beta (H57-597), CD8 (53-6.7), CD4 (RM4-5) were obtained from eBioscience/Thermo Fisher. Dead cells were excluded from analysis by staining with Fixable Viability dye from eBioscience/Thermo Fisher. Data were acquired on a ZE5 flow cytometer (Bio-Rad) equipped with Everest Software. Analysis were performed using FlowJo software (TreeStar). For flow analysis, anti-mouse PTPRS (MEDIMABS) was labeled with AlexaFluor 647 using Mix-n-Stain™ antibody labeling kit (Sigma-Aldrich).

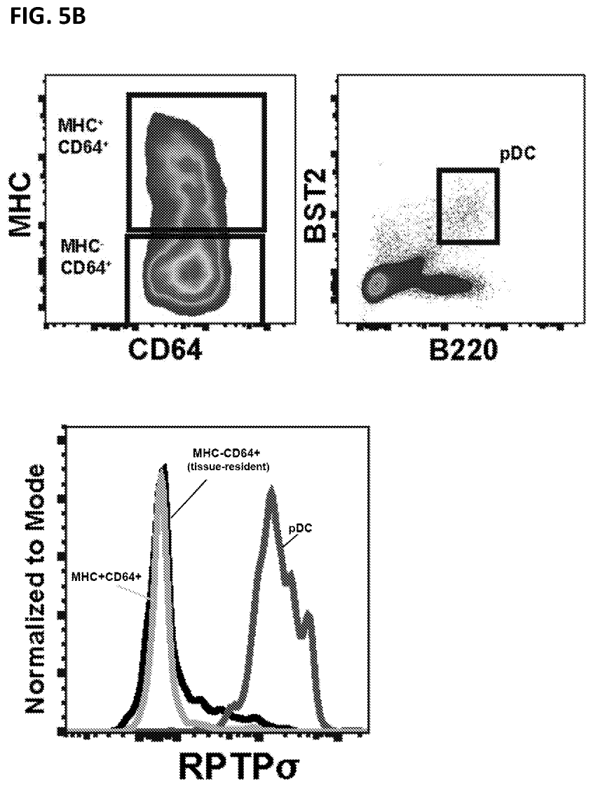

As shown in FIG. 5A, neither classical (Ly6C+CD43−), intermediate (Ly6C+CD43+), or non-classical (Ly6C−CD43+) circulating monocytes (blood monocytes) showed significant expression of RPTPσ. The expression of RPTPσ in plasmacytoid dendritic cells (pDC) is shown for comparison. As shown in FIG. 5B, that neither classical (Ly6C+CD43−), intermediate (Ly6C+CD43+), or non-classical (Ly6C−CD43+) joint macrophages (ankle macrophages) showed significant expression of RPTPσ. The expression of RPTPσ in plasmacytoid dendritic cells (pDC) which are known to express high levels of RPTPσ is shown for comparison.

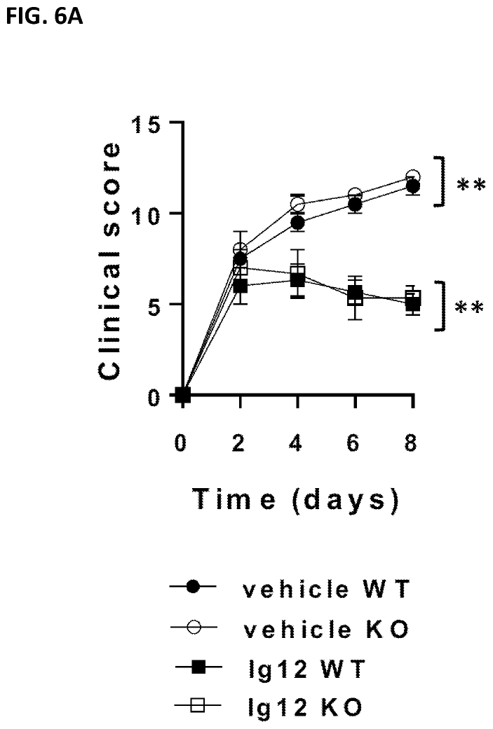

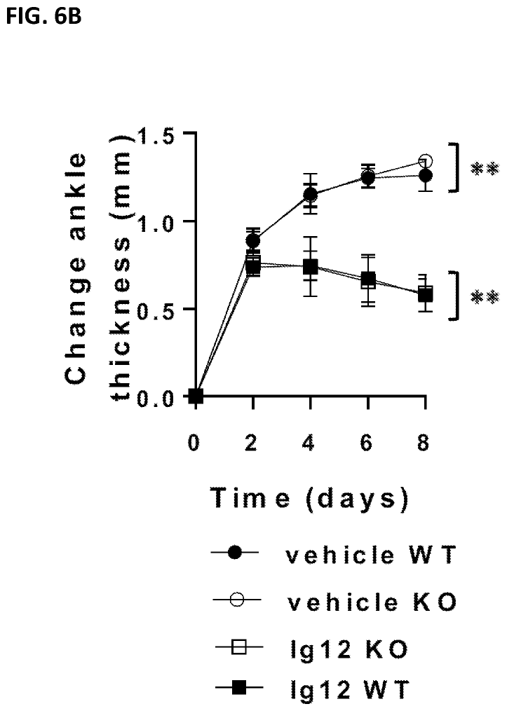

In order to dissect the effect of Ig1&2 on FLS versus radiosensitive bone marrow-derived cells in vivo, we compared the efficacy of Ig1&2 at attenuating STIA in CD45.1 congenic mice subjected to lethal irradiation (>1000 Rad) and bone-marrow transplantation from CD45.2 WT or PTPRS KO mice (FIGS. 6A and 6B). To generate bone marrow chimeras, male BALB/cByJ (CD45.1 congenic) mice were lethally irradiated with 2 doses of 550 Rads using an RS 2000 Biological irradiator and subsequently administered bone-marrow from male PTPRS WT or KO congenic CD45.2 donor mice. KB×N serum was administered to induce arthritis 8 weeks post-irradiation. After 7 weeks, chimerism was verified by flow cytometry by staining for the appropriate CD45 allele (anti-CD45.1 and anti-CD45.2; eBioscience). The percentage of engraftment in recipient mice was greater than 95%. His-Ig1&2 in Tris-buffered saline (TBS) or vehicle control (TBS) were administered as described in the results. KO (KO) versus wild type (WT) mice (the His-tagged Ig1&2 “in use” construct was utilized) showed the same responsiveness to Ig1&2 in the STIA model. Bone-marrow-reconstituted mice were injected retro-orbitally (r.o.) with 500 μg Ig1&2 (n=3 per group) or vehicle (n=2 per group) on days 0, 2, 4 and 6 post-serum transfer. The clinical score for this experiment is shown on FIG. 6A, and FIG. 6B shows the change of ankle thickness (in mm). In both figures, means+/−s.e.m. are shown, and data were analyzed using the two-way analysis of variance (ANOVA, **, P<0.01). Mice reconstituted with WT or KO bone marrow displayed identical arthritis severity once subjected to STIA. Furthermore, Ig1&2 displayed identical anti-arthritic action in the two groups of mice. STIA was initiated 8 weeks post-irradiation. Mice were with either vehicle or 0.5 mg of His-Ig1&2 by retro orbital (r.o.) injection once every other day for a total of 4 treatments. Groups WT BM control (n=4), WT BM His-Ig1&2 (n=5), KO BM control (n=6), KO BM His-Ig1&2 (n=7). ** P<0.01, by Mann Whitney.

We compared the efficacy of therapeutic Ig1&2 doses as monotherapy and in combination with a therapeutic dose of TNF inhibitor in reversal of mouse collagen-induced arthritis (CIA), which is driven by the adaptive immune system and considered as the gold-standard for preclinical therapeutic development for RA. The collagen-induced arthritis (CIA) model was performed. Briefly, 8-10 week-old male DBA/1J were immunized with 100 chicken type II collagen (Condrex) emulsified in Freund's adjuvant containing 50 μg of Mycobacterium tuberculosis (H37Ra, ATCC 25177) (CFA, Sigma-Aldrich). After 28 days, mice were boosted with 100 μg chicken type II collagen emulsified in incomplete Freund's adjuvant (IFA; Sigma-Aldrich). Arthritis was assessed by clinical scoring as described above.

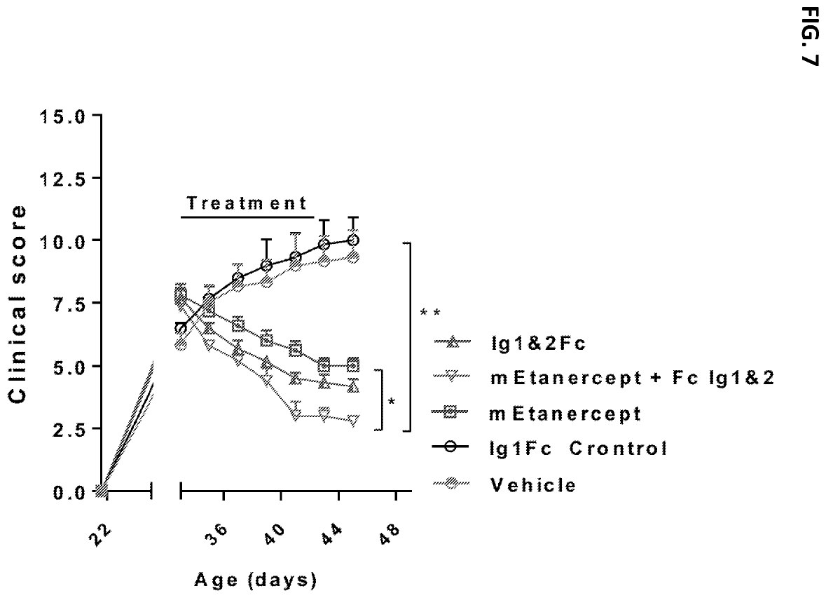

Preparation of recombinant PTPRS 6×HisIg1&2 (here called His-Ig1&2) was described in (Doody K M, et al., “Targeting phosphatase-dependent proteoglycan switch for rheumatoid arthritis therapy. Sci Transl Med. 2015 May 20; 7(288):288ra76). Preparation of human IgG1-Fc-fused Ig1&2 (here called Fc-Ig1&2) was contracted to LakePharma (USA). Murine TNF-blocking biologic p75TNFR:Fc (mEtanercept) was obtained from Amgen through the Amgen Extramural Research Alliance Program. Fc-Ig1&2 in 20 mM Tris with 120 mM NaCl or human IgG1-Fc control and mEtanercept was administered according to the schedule described in the results. A 4 mg/kg dose of mEtanercept, that has been shown to be the effective was used. Combination of Fc-Ig1&2 and a therapeutic dose of mEtanercept led to enhanced reduction of clinical arthritis severity. 6 mice per group were respectively intraperitoneally injected days 34, 36 and 38 post immunization with IgG1Fc (control including Fc alone), vehicle, 0.5 mg Fc-Ig1&2 “in use”+4 mg/kg mEtanercept and 0.5 mg Fc-Ig1&2. Arthritis was assessed every 2 days by clinical scoring. Means+s.e.m. are shown. FIG. 7 shows that Fc-Ig1&2 was effective at reversing collagen-induced arthritis (CIA) as monotherapy or in combination with TNF inhibitor mEtanercept (p75mTNFr:Fc, a mouse equivalent of Etanercept), and that the combination of therapeutic doses of Ig1&2 and mEtanercept showed significantly higher efficacy than therapeutic doses of TNF inhibitor alone. In these experiments, the mice received primary immunization at day 0 and were boosted at day 21. 6 mice per group were respectively intraperitoneally injected days 34, 36 and 38 post immunization with IgG1Fc (control including Fc alone), vehicle, 0.5 mg Fc-Ig1&2 “in use”+4 mg/kg mEtanercept and 0.5 mg Fc-Ig1&2. Arthritis was assessed every 2 days by clinical scoring. Means+s.e.m. are shown.

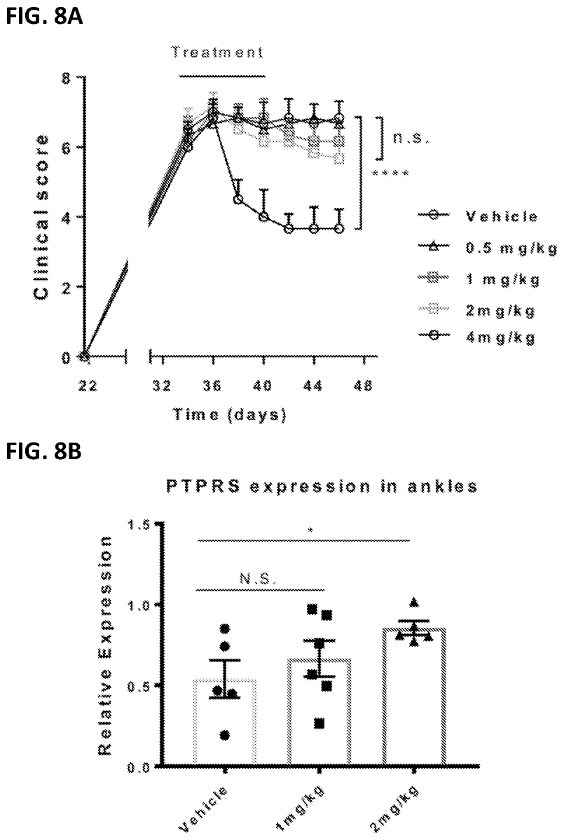

A dose-response of Etanercept in CIA showed that a subtherapeutic 2 mg/kg dose of mEtanercept is sufficient to enhance Ptprs expression in arthritic joints of mice. 6 CIA mice per group were respectively intraperitoneally injected at days 34, 36 and 38 post immunization with vehicle or mEtanercept (4 mg/Kg, 2 mg/Kg, 1 mg/Kg, and 0.5 mg/Kg). Arthritis was assessed every 2 days by clinical scoring. Means+−s.e.m. are shown. As shown in FIG. 8A, the evolution of the clinical score for mice that received primary immunization at day 0 and were boosted at day 21. A number of 6 CIA mice per group were respectively intraperitoneally injected at days 34, 36 and 38 post immunization with vehicle or mEtanercept (4 mg/kg, 2 mg/kg, 1 mg/kg, and 0.5 mg/kg). FIG. 8B, shows PTPRS expression in ankles by qPCR normalized to expression of GAPDH.

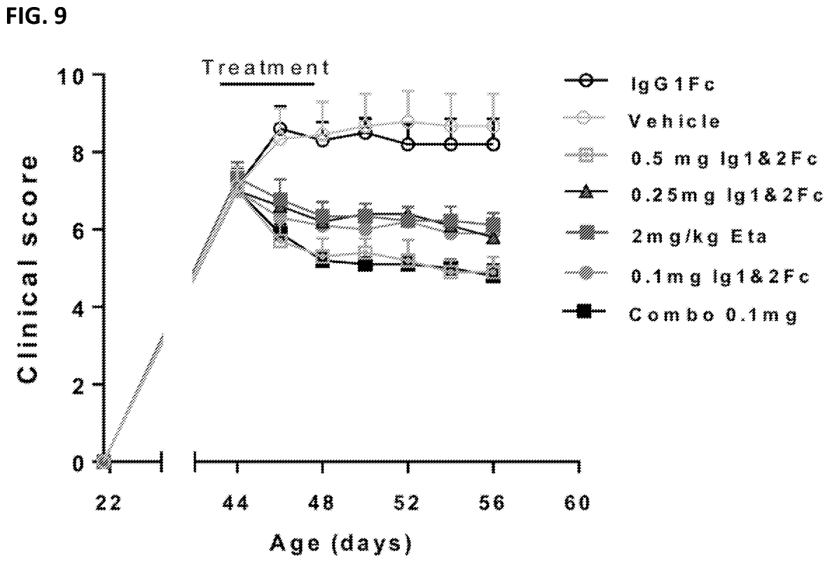

We assessed the efficacy of subtherapeutic doses (0.1 and 0.25 mg) of Ig1&2 administered as monotherapy and in combination with a subtherapeutic dose (2 mg/kg) of TNF inhibitor in reversal of mouse collagen-induced arthritis (CIA). Combination of a subtherapeutic dose of Fc-Ig1&2 and a subtherapeutic dose of mEtanercept led to a surprising reduction of clinical arthritis severity. 10 mice per group were respectively intraperitoneally injected at days 44, 46 and 48 post immunization with: IgG1-Fc (control including Fc alone), vehicle (see above), 0.5 mg Fc-Ig1&2, 0.25 mg Fc-Ig1&2, 2 mg/Kg mEtanercept, 0.1 mg Fc-Ig1&2, combo 0.1 mg (0.1 mg Fc-Ig1&2+2 mg/kg mEtanercept) Arthritis was assessed every 2 days by clinical scoring. titration of Fc-Ig1&2 in combination, or not, to Etanercept. As shown in FIG. 9, the treatment with Fc-Ig1&2 did not affect antibody titer generation after immunization with collagen in CFA and boost in IFA. The data provides that Ig1&2 does not affect T-cell dependent B cell antibody responses in mice and that the efficacy of Ig1&2 in CIA is not due to depression of adaptive immune responses. In this experiment, the mice received primary immunization at day 0 and were boosted ad day 21. 10 mice per group were respectively intraperitoneally injected at days 44, 46 and 48 post immunization with either IgG1-Fc (control including Fc alone), vehicle, 0.5 mg Fc-Ig1&2, 0.25 mg Fc-Ig1&2, 2 mg/Kg mEtanercept, 0.1 mg Fc-Ig1&2, or 0.1 mg Fc-Ig1&2+2 mg/kg mEta (Combo 0.1 mg). Arthritis was assessed every 2 days by clinical scoring.

Combinations of Ig1&2 and a TNF inhibitor (e.g., mouse equivalent of etanercept) lead to increased efficacy compared to each agent alone (FIG. 7) in mice affected by CIA and that combination Ig1&2 and of the TNF inhibitor at a level below its effective dose (FIGS. 8 and 9), demonstrating synergistic effect.

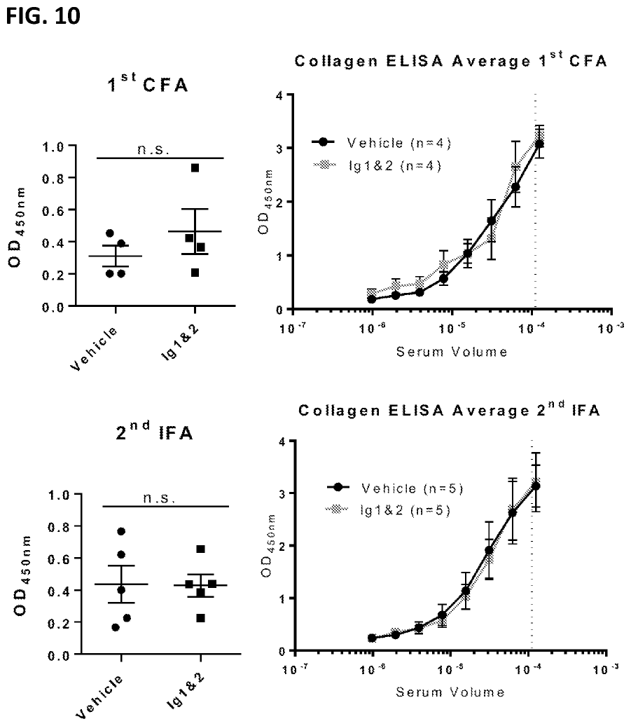

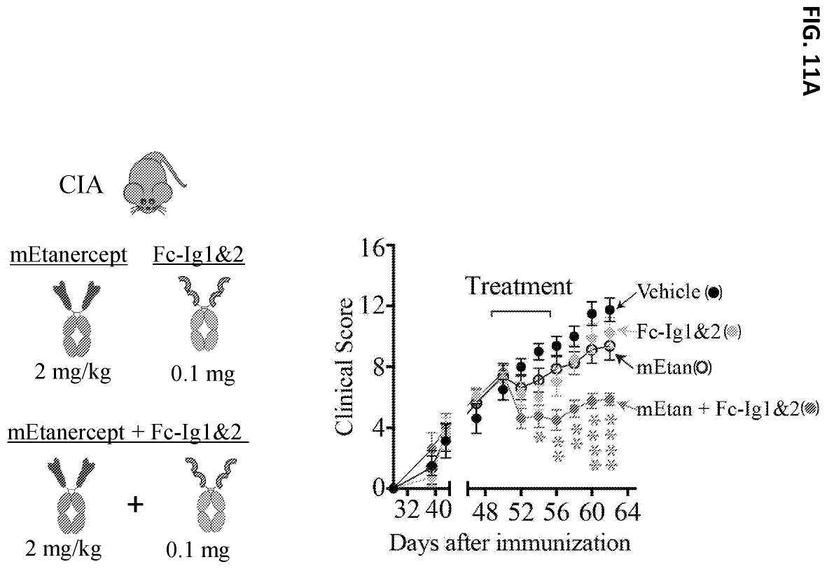

Treatment of mice with a therapeutic dose (0.5 mg i.p.) of Fc-Ig1&2 around the primary of secondary anti-collagen immunization for CIA induction does not influence induction of anti-collagen antibodies. In one cohort, mice were immunized with type II collagen in CFA, and administered 0.5 mg Ig1&2 or vehicle i.p. every 2 days for a total of 4 injections starting 2 days prior to the immunization. Serum was obtained at the end of the treatment for assessment of anti-collagen antibody titers. In another cohort, mice were immunized with type II collagen in CFA and boosted with collagen in IFA (day 21). Mice were administered 0.5 mg Ig1&2 or vehicle i.p. every 2 days for a total of 4 injections starting 2 days prior to the boost immunization. Serum was obtained at the end of the treatment for assessment of anti-collagen antibody titers. Serum anti-collagen antibody levels were assessed by ELISA. Anti-collagen antibody levels in sera of mice immunized with collagen were measured by enzyme-linked immunosorbent assay (ELISA). Briefly, low-binding 96 multi-well plates (Costar) were coated with type II chicken sternal collagen (1 μg/ml; Sigma). Sera were incubated in serial dilutions, and IgG binding to collagen was detected using a biotinylated anti-mouse IgG antibody (Jackson Laboratories) as well as anti-mouse IgG1, IgG2a, IgG2b and IgG3 antibodies (Southern Biotech) followed by incubation with extravidin-HRP (Sigma) and exposure with 3,3′,5,5′-tetramethylbenzidine (TMB) substrate. Plate absorbance was read at 450 nm using a Tecan Infinite M1000 plate-reader. As shown in FIG. 10, the treatment with Fc-Ig1&2 did not affect antibody titer generation after immunization with collage in CFA and boost in IFA. The data showed that Ig1&2 does not affect T-cell dependent B cell antibody responses in mice and that the efficacy of Ig1&2 in CIA was not due to depression of adaptive immune responses.

Therefore, and consistent with the non-immunological mechanism of action of Ig1&2, these results suggest that the titers of pathogenic antibodies generated after immunizing mice with collagen for CIA was unaffected by treatment with Fc-Ig1&2.

### Example 3. Synergistic Reversal of Arthritis by Synoviocyte-Targeted Therapy and TNF Immunomodulation

Therapeutic synergy between subtherapeutic doses of Fc-Ig1&2 and mEtanercept in CIA shown by treating mice with severe arthritis (clinical score at least 8) with doses of Ig1&2 (0.1 mg) and mEtanercept (2 mg/kg) which are ineffective at reversing severe arthritis as monotherapy. CIA was induced and scored as described above. Fc-Ig1&2 and mEtanercept were obtained and injected as described above. For histological scoring of mouse arthritic joints, whole hind paws were fixed in 10% formalin, decalcified, trimmed and embedded. Sections were prepared from tissue blocks and stained with H&E, Safranin-O or Toluidine blue (HistoTox). Histopathological scoring was performed as described. Briefly, joints of arthritic mice were assigned scores of 0-4 for inflammation based on H&E staining, according to the following criteria: 0=normal; 1=minimal infiltration of inflammatory cells in periarticular area; 2=mild infiltration; 3=moderate infiltration; and 4=marked infiltration. Joints of arthritic mice were given scores of 0-4 for bone resorption based on H&E staining, according to the following criteria: 0=normal; 1=minimal (small areas of resorption, not readily apparent on low magnification); 2=mild (more numerous areas of resorption, not readily apparent on low magnification, in trabecular or cortical bone); 3=moderate (obvious resorption of trabecular and cortical bone, without full thickness defects in the cortex; loss of some trabeculae; lesions apparent on low magnification); and 4=marked (full-thickness defects in the cortical bone and marked trabecular bone loss). Cartilage depletion was identified by diminished Safranin 0 or Toluidine blue staining of the matrix and was scored on a scale of 0-4, where 0=no cartilage destruction (full staining with Safranin 0), 1=localized cartilage erosions, 2=more extended cartilage erosions, 3=severe cartilage erosions and 4=depletion of entire cartilage. Histologic analyses were performed in a blinded manner. Images of whole ankles were acquired using the Zeiss Axioscan.Z1 (Zeiss) slide scanner and analyzed using Zen software (Zeiss)

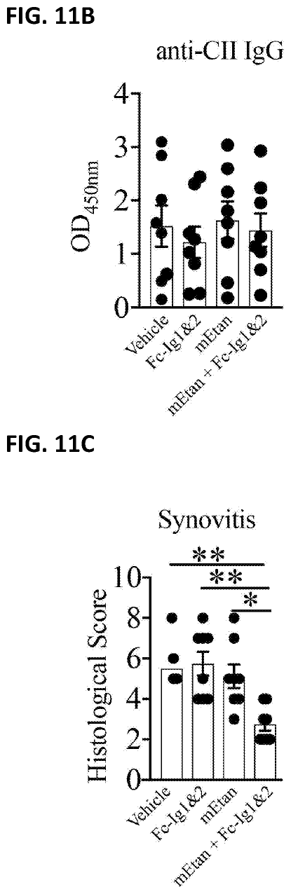

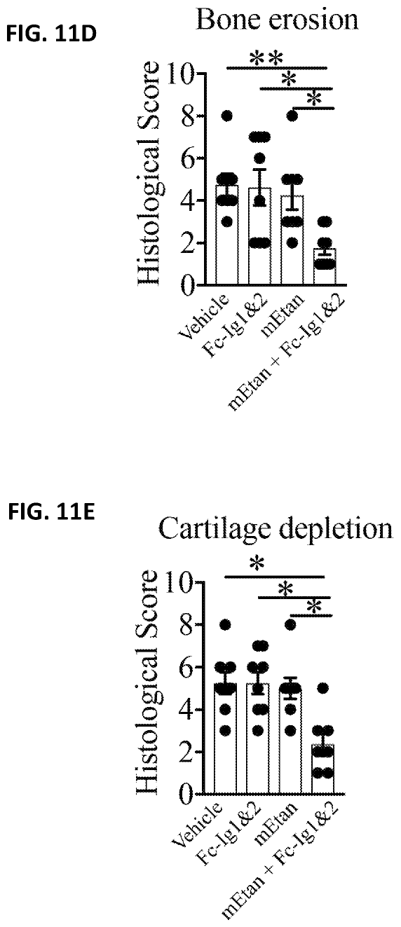

The combined treatment with subtherapeutic doses of Ig1&2 and mEtanercept results in synergistic reversal of CIA. CIA was induced in DBA/1 male mice by two immunizations (primary [d 0] and boost [d 28]) with 100 microg of chicken type II collagen. Arthritic mice were treated with vehicle (PBS), Fc-Ig1&2 (0.1 mg), mEtanercept (2 mg/Kg) or a combination of Fc-Ig1&2 (0.1 mg) & mEtanercept (2 mg/kg) every 48 h for the indicated time period. (FIG. 11A) N=8/group. Arthritis was assessed every 2 days by clinical scoring. (FIG. 11B) Synergistic reversal of arthritis by combined Ig1&2 and mEtanercept treatment was not associated with decreased titers of anti-collagen antibodies. Panel shows anti type II collagen IgG antibodies in the serum of mice in A at the end of the experiment using ELISA. (FIG. 11C-E) Synergistic reversal of arthritis by combined Ig1&2 and mEtanercept treatment was associated with significant improvement of histological inflammation and cartilage and bone erosion scores. Notice that monotherapies with Ig1&2 and mEtanercept were unable to improve the histological scores consistent with the lack of reversal of clinical arthritis. Figures show histopathological evaluation of synovitis (FIG. 11C) bone erosion (FIG. 11D) and cartilage depletion (FIG. 11E) of mice in FIG. 11A. Graphs show mean+−s.e.m. *P<0.05, **P<0.01, ***P<0.001, ****P<0.0001, by two-way ANOVA (FIG. 11A, vs Vehicle group) or one-way ANOVA (FIG. 11C-E)

### Example 3. PTPRS is Enriched in Lining Layer FLS in the RA Synovium

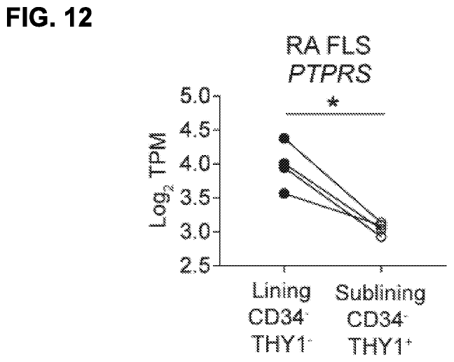

In order to further establish PTPRS as a target for FLS-directed therapy in RA we examined the expression profile of PTPRS in the rheumatic synovium. The enriched expression of PTPRS in synovial lining RA FLS (CD34-THY1-(2 9)) when compared to subliming RA FLS (CD34-THY1+(29)) was shown in RNA-seq data from RA FLS isolated from the synovial tissue of RA patients (FIG. 12).

Fc-Ig1&2 Shows High Accumulation in Arthritic Paws and Suppresses Experimental Arthritis

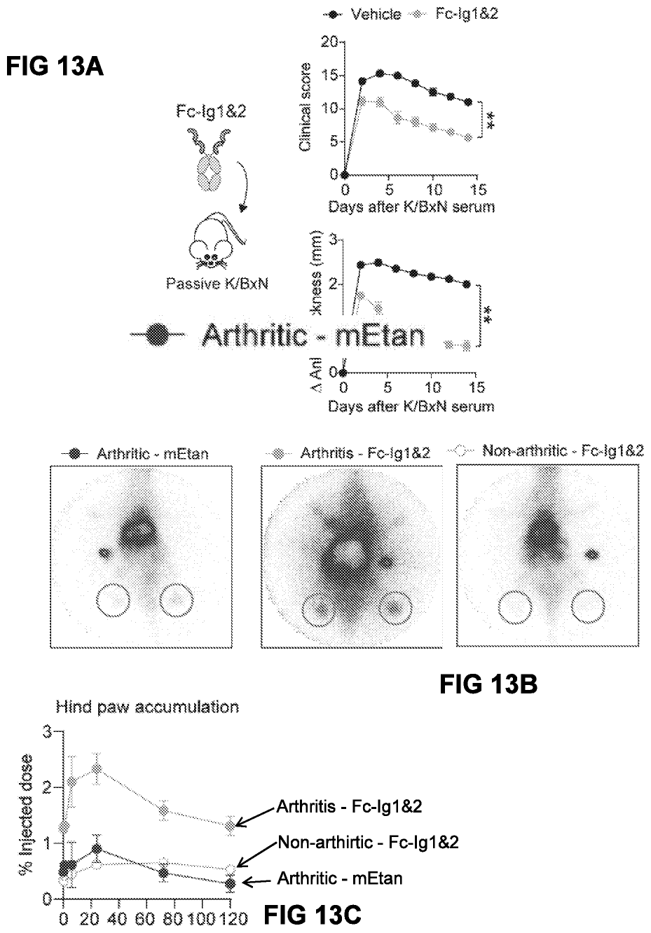

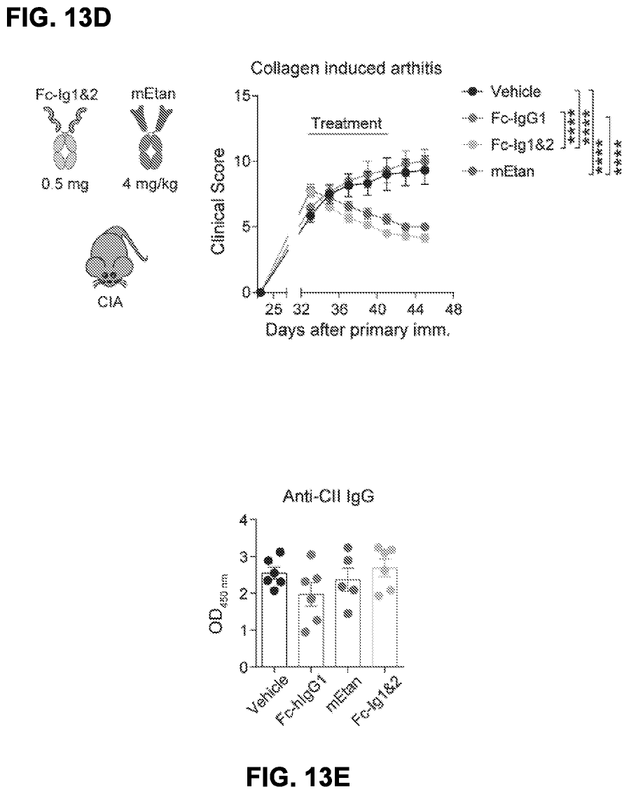

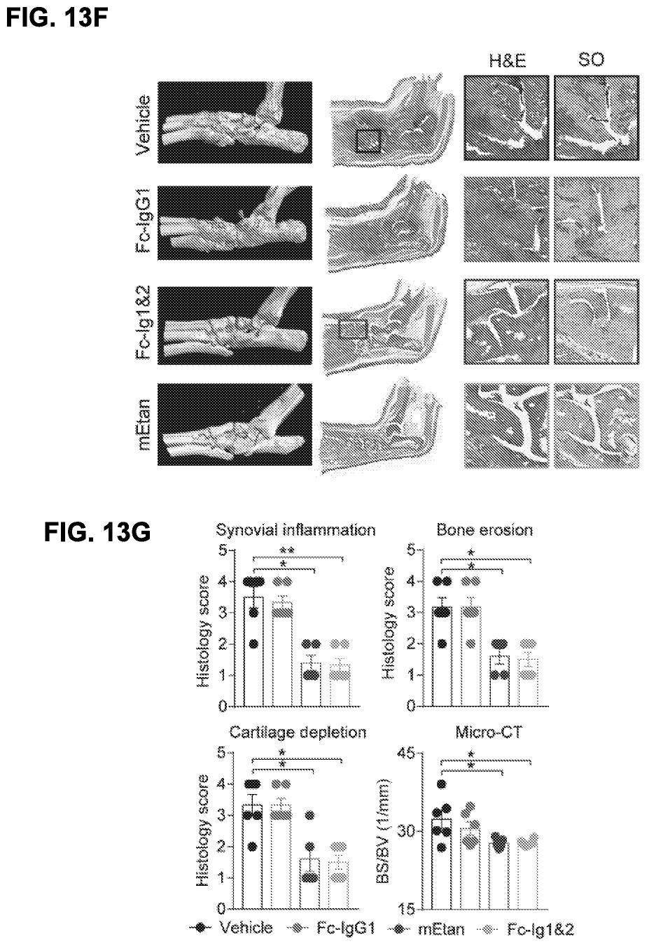

Next, we sought to characterize the efficacy of Fc-Ig1&2 in experimental mouse arthritis. Similar to as shown for 6×His-Ig1&2 (Doody K M, et al., “Targeting phosphatase-dependent proteoglycan switch for rheumatoid arthritis therapy. Sci Transl Med. 2015 May 20; 7(288):288ra76), we found that Fc-Ig1&2 was highly effective (FIG. 13A). Mice with established STIA were injected with 111-indium (¹¹¹In)-labeled Fc-Ig1&2. Radiolabeling of Fc-Ig1&2 did not affect the function of the protein. As controls, we used ¹¹¹In-labeled mEtanercept and injected ¹¹¹In-Fc-Ig1&2 in non-arthritic mice. As shown in FIGS. 13B & C, Fc-Ig1&2 showed significant increased accumulation in the joints of arthritic mice in comparison to mEtanercept. No accumulation of ¹¹¹In-Fc-Ig1&2 in mouse brains and eyes was observed, suggesting that Fc-Ig1&2 does not cross over the blood brain barrier.

Administration of Fc-Ig1&2 to arthritic CIA mice caused a significant reduction in disease severity, comparable to treatment with the murine homolog of Etanercept (murine p75TNFR:Fc, here called mEtanercept), using a 4 mg/kg dose that has been shown to be effective in CIA (FIG. 13D). The efficacy of Fc-Ig1&2 in CIA was not associated with decreased production of anti-collagen antibodies (FIG. 13E). Fc-Ig1&2 administration also protected mice from cartilage damage and bone erosions as assessed by histopathology and micro-computed tomography (microCT) analysis of joints (FIG. 13F-G). Together, these results suggest that Ig1&2 has a favorable biodistribution profile and exerts a therapeutic effect in multiple models of arthritis driven by the innate and/or adaptive immune system.

Ig1&2 Attenuates Inflammatory Arthritis without Suppressing the Innate Immune System

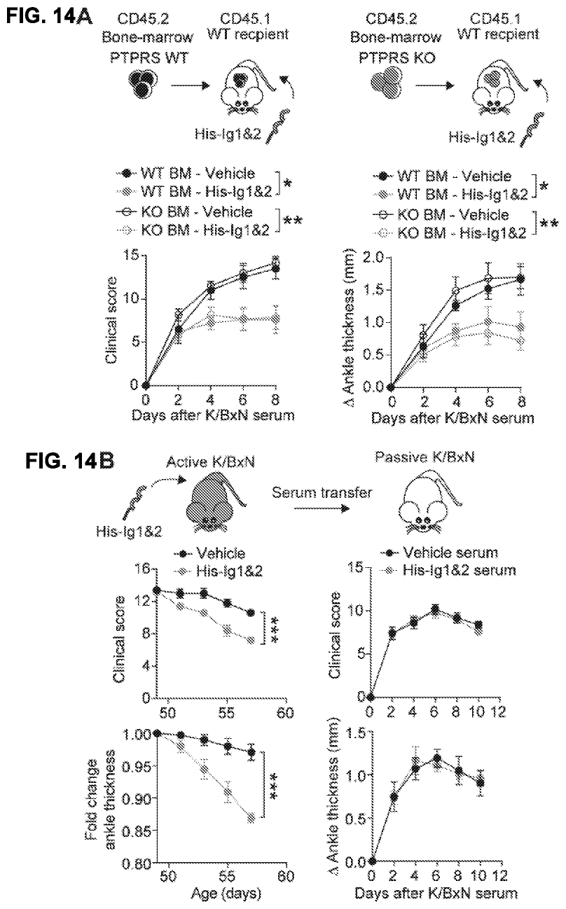

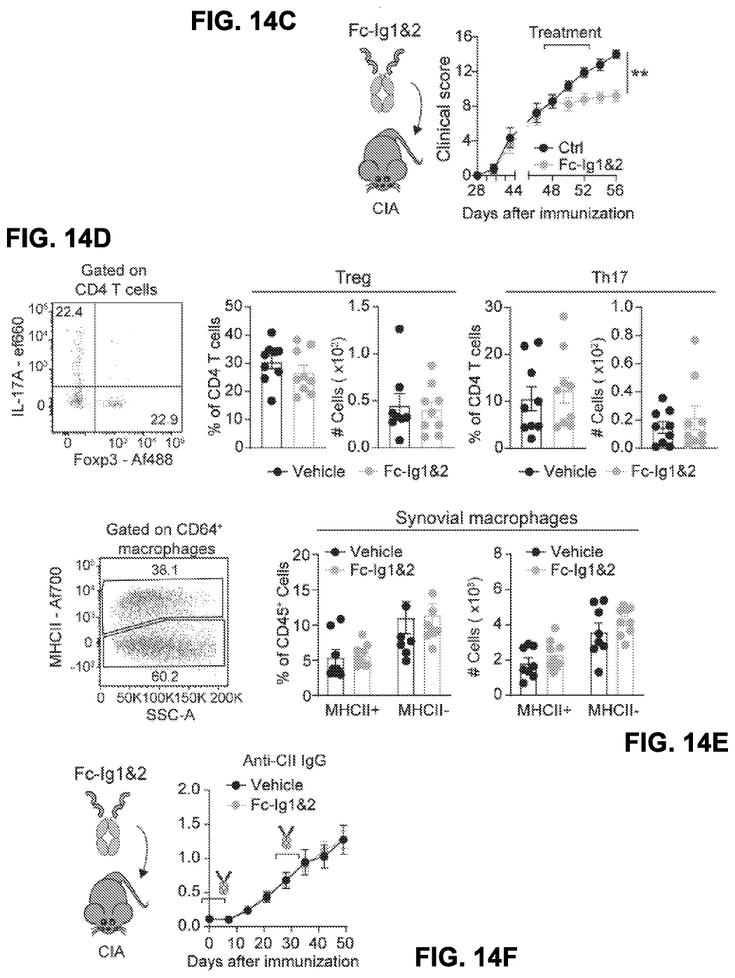

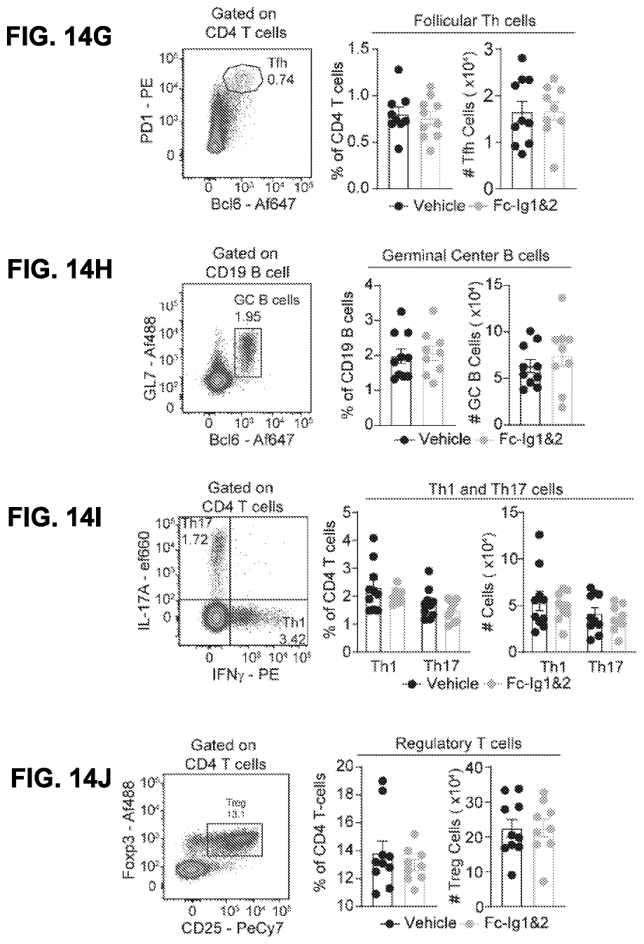

We sought to characterize if the therapeutic effect of Ig1&2 is mediated through the suppression of the innate or adaptive immune system. Neither classical (Ly6C⁺CD43⁻), intermediate (Ly6C⁺CD43⁺), or non-classical (Ly6C⁻CD43⁺) circulating monocytes showed detectable expression of PTPRS in STIA mice. Furthermore, PTPRS was absent on MHCII⁺CD64⁺ macrophages and expressed at low levels in a small subset of MHCII⁻CD64⁺ macrophages isolated from arthritic ankles of STIA mice. Next, in order to dissect the effect of Ig1&2 on FLS versus radiosensitive bone marrow-derived cells in vivo, we compared the efficacy of Ig1&2 at attenuating STIA in CD45.1 congenic mice subjected to lethal irradiation (>1000 Rad) and bone-marrow transplantation from CD45.2 WT or PTPRS KO mice (FIG. 14A). Mice reconstituted with WT or KO bone marrow displayed identical arthritis severity once subjected to STIA. Furthermore, Ig1&2 displayed identical anti-arthritic action in the two groups of mice. To complete our assessment of whether innate immunity mediates the anti-arthritic action of Ig1&2, we turned to pDC, which express high levels of PTPRS. In order to assess whether pDC contribute to the anti-arthritic action of Ig1&2 in vivo, we depleted pDC using an anti-PDCA-1 antibody prior to subjecting mice to STIA (FIG. 14C). We found no difference in arthritis development between pDC-depleted and non-depleted mice and the anti-arthritic effect of Ig1&2 was maintained in pDC-depleted mice.

To further rule out any potential effects of Ig1&2 on activated macrophages, we also evaluated macrophage polarization and bacterial phagocytosis. First, Fc-Ig1&2 was added during the polarization of bone marrow derived macrophages (BMDM) into either M1 or M2 effector phenotypes. For both effector phenotypes, the addition of Fc-Ig1&2 did not affect the upregulation of characteristic transcription factors or cytokines (Fig. S4A, B). Similarly, Fc-Ig1&2 did not alter the phagocytosis of either E. Coli or S. aureus by M0, M1 or M2 macrophages (Fig. S4C-D). This suggests that Ig1&2 does not alter functions of innate immune cells and that its therapeutic in mouse arthritis is not mediated through suppression of the innate immune system.

The Therapeutic Effect of Ig1&2 in CIA is not Mediated Through the Adaptive Immune System

To assess whether Ig1&2 exerts an effect on arthritis development through an action on the adaptive immune system, we administered Ig1&2 to arthritic K/B×N transgenic mice which develop spontaneous arthritis starting at 6-7 weeks of age. We observed a significant reversal of spontaneous arthritis in these mice after treatment with Ig1&2 (FIG. 14B). However, when sera of Ig1&2-treated K/B×N mice were transferred into WT recipient mice to induce STIA, we did not observe any difference in the arthritogenicity of sera from the Ig1&2- vs vehicle-treated K/B×N mice. Thus, reversal of spontaneous K/B×N arthritis by Ig1&2 is not mediated by decreased titers of arthritogenic antibodies.

Next, we used mouse CIA to further determine the effect of Fc-Ig1&2 on the adaptive immune system during arthritis. The therapeutic efficacy of Fc-Ig1&2 was not associated with an altered accumulation or expansion of regulatory T cells (Tregs) or Th17 cells in arthritic ankles of CIA mice (FIG. 14C-D). Consistent with the STIA data, treatment with therapeutic doses of Fc-Ig1&2 also did not alter the number or frequency of MHCII⁺CD64⁺ and MHCII⁻CD64⁺ macrophages in the same ankles (FIG. 14E). Furthermore, the therapeutic effect of Fc-Ig1&2 was not associated with an alteration in the expansion of naïve and effector CD4 T cells or the expansion of the CD4 T cell effector populations Th1 or Th17 cells in the spleen and lymph nodes of arthritic CIA mice. Similarly, we did not observe any effects on germinal center (GC) B cell or CD4 T follicular helper (Tfh) cell numbers, which are important for the generation of pathogenic autoantibodies in the CIA model. Lastly, there was no change in regulatory T cell (Treg) numbers, or in the expression of CTLA4 by Tregs, in arthritic CIA mice after treatment with Fc-Ig1&2. Together, these results suggest that the therapeutic action of Fc-Ig1&2 in CIA is not mediated through an effect on CD4 effector populations or germinal center B cells.

Ig1&2 does not Alter Adaptive Responses to Immunization

To determine if Ig1&2 could potentially affect the immune response towards immunizations—which are affected by immunosuppressive DMARDs—we next treated DBA1/J mice with Fc-Ig1&2 during the primary and boost immunization against type-II collagen and followed the generation of anti-collagen type-II antibodies. We did not observe any effect of Fc-Ig1&2 on overall titers of anti-collagen IgG antibodies (FIG. 14F) or the titers of anti-collagen IgG subclasses IgG1, IgG2a, IgG2b and IgG3. We also did not observe any effect of Fc-Ig1&2 on the frequency and numbers of Tfh cells or GC B cells (FIG. 14G-H). Together, these results suggest that Ig1&2 does not affect humoral responses to immunization with type-II collagen.

Ig1&2 does not Alter the Polarization of CD4 T Cells

PTPRS-KO CD4 T cell show enhanced polarization into Th1 and Th17 cells. To determine if Ig1&2 affects CD4 T cell polarization during collagen immunizations, we evaluated CD4 T cell populations in mice treated with Fc-Ig1&2 during primary and boost immunizations. We did not find any skewing of population of naïve and effector CD4 T cells in the spleen and lymph nodes of Fc-Ig1&2-treated mice. Also, we did not observe any alterations of the numbers of Th1, Th17 or Tregs in either lymph nodes (FIG. 14I-J) or spleen of the same mice. Tregs from lymph nodes and spleen of mice treated with either Fc-Ig1&2 or vehicle displayed similar levels of CTLA4 expression. To further dissect the effect of Ig1&2 on the polarization of CD4 T cells, we performed in vitro polarization assays stimulated by plate-bound antibodies or antigen presenting cells (APCs). Similar to what we observed in vivo, Ig1&2 did not alter the polarization of CD4 T cells isolated from BALB/c or DBA1/J mice into Th1, Th17 or Treg cells regardless of the presence or absence of APCs. Together, these results suggest that Ig1&2 does not affect the polarization of CD4 T cells in vitro or in vivo and support the idea that the therapeutic effect of Fc-Ig1&2 is unlikely to be mediated through immunosuppression.

TNF Regulates PTPRS Expression in RA FLS Through the PI3K/GSK3β/USF2 Pathway

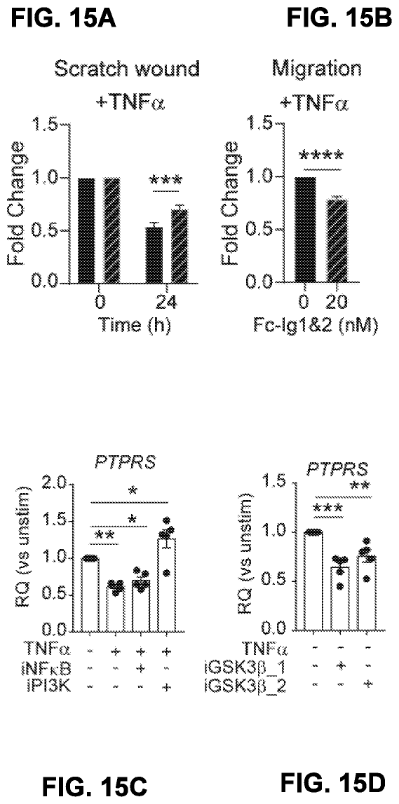

Further evaluation of the epigenomic landscape of the PTPRS locus in RA FLS identified histone marks for both active promoters (H3K4me3), primed enhancers (H3Kme1) and repressive marks (H3K27me3) around exon 1 of the PTPRS gene. Using the ENCODE database and the UCSC human genome browser we identified potential binding sites for two downstream targets of GSK3β, upstream stimulatory factor (USF)-1 and USF2, in the promoter region of PTPRS suggesting an involvement of these two transcription factors in the regulation of PTPRS expression (FIG. 15A). This was further supported by a positive correlation between the expression of either USF1 or USF2 with PTPRS in RA and OA FLS as analyzed by RNA-seq. By using siRNA-mediated knock-down of USF2, we found that USF2 indeed promotes the expression of PTPRS in RA FLS (FIG. 15B). Using chromatin immunoprecipitation (ChIP), we could further confirm that USF2 binds the promoter region of PTPRS in resting RA FLS, and that such interaction was significantly reduced after stimulation with TNF (FIG. 15C). Finally, using a luciferase reporter construct encompassing the PTPRS promoter region, we show that overexpression of USF2 causes a strong activation of the PTPRS promoter (FIG. 15D). Together, these results suggest that expression of PTPRS in RA FLS is regulated by TNF via a PI3K/GSK3β/USF2 pathway.

We determined whether TNF modulates PTPRS expression. RA (N=13) and OA (N=13) FLS were serum-starved for 24h and stimulated with different concentrations of TNF for 12h.

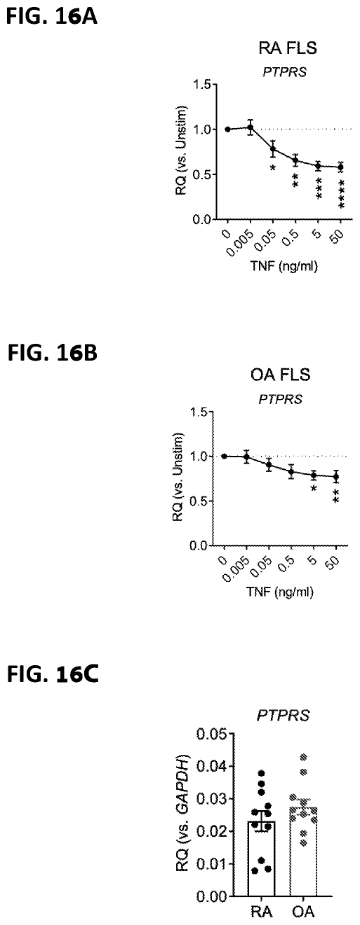

As shown in FIG. 16A-B, stimulation with TNF caused concentration and time-dependent down-regulation of PTPRS expression in RA FLS and OA FLS at the mRNA level although no difference in the basal mRNA expression of PTPRS between RA and OA FLS could be detected (FIG. 16C) Data were analyzed using one-way analysis of variance (ANOVA, *P<0.05, **P<0.01,***P<0.001 ****P<0.0001)).

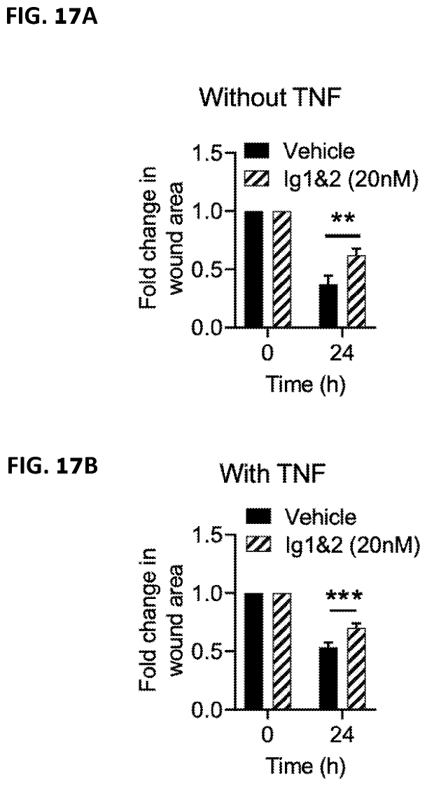

Although the expression of PTPRS is downregulated by TNF, TNF-treated RA FLS retain responsiveness to Ig1&2 in vitro motility assays. FLS (N=9) monolayers were serum-starved before scratch-wounding and stimulation with 1% FBS in the presence of Vehicle, 50 ng/mL TNF and 20 nM Ig1&2. Wound with was measured at 24h and normalized to wound with at 0h. Data were analyzed using the two-way analysis of variance (ANOVA), **P<0.01, ***P<0.001). FIG. 17A shows results in the absence of TNF and FIG. 17B shows results in the presence of TNF stimulation.

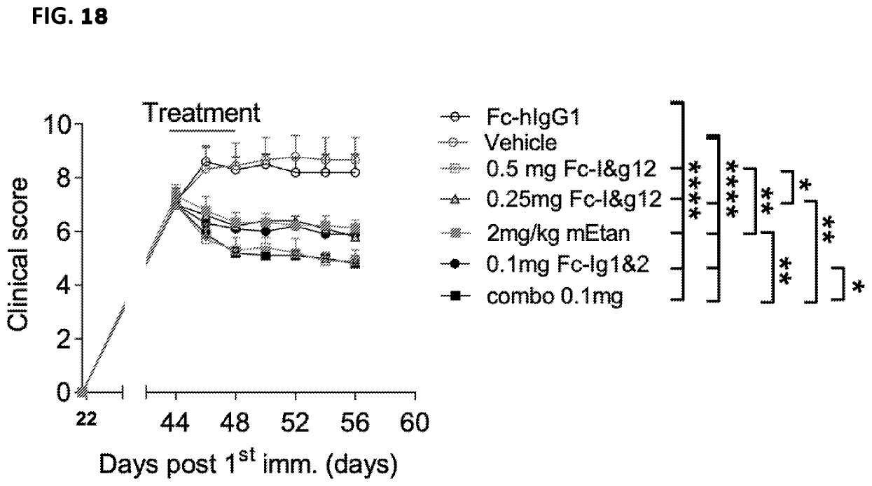

We assessed the efficacy of subtherapeutic doses (0.1 and 0.25 mg) of Ig1&2 administered as monotherapy and in combination with a subtherapeutic (2 mg/kg) dose of TNF inhibitor in reversal of mouse collagen-induced arthritis (CIA). Combination of subtherapeutic dose of Fc-Ig1&2 and a subtherapeutic dose of mEtanercept led to a surprising reduction of clinical arthritis severity (FIG. 18). Male DBA/J mice/group received a primary immunization against type-II collagen at day 0 and were boosted on day 28. Mice were treated with human IgG1 Fc control (Fc-hIgG1, N=10), Vehicle (N=9), 2 mg/kg murine etanercept (mEtan, N=9), 0.5 mg Fc-Ig1&2 (N=10), 0.25 mg Fc-Ig1&2 (N=10), 0.1 mg Fc-Ig1&2 (N=10), 2 mg/kg mEtan+0.1 mg Fc-Ig1&2 (combo, N=10) by intraperitoneal injections on days 44, 46 and 48 post primary immunization. Arthritis was assessed every 2 days by clinical scoring. Data were analyzed using the two-way analysis of variance (ANOVA), *P<0.05, **P<0.01, ****P<0.0001))

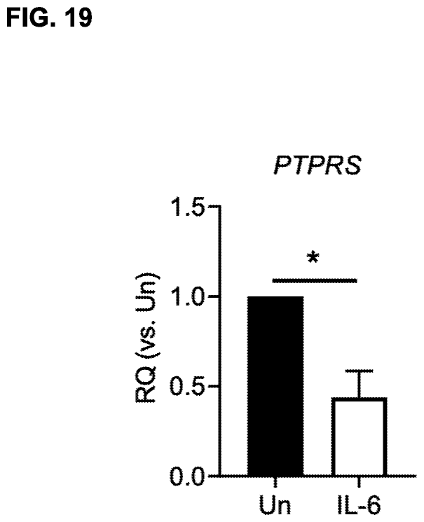

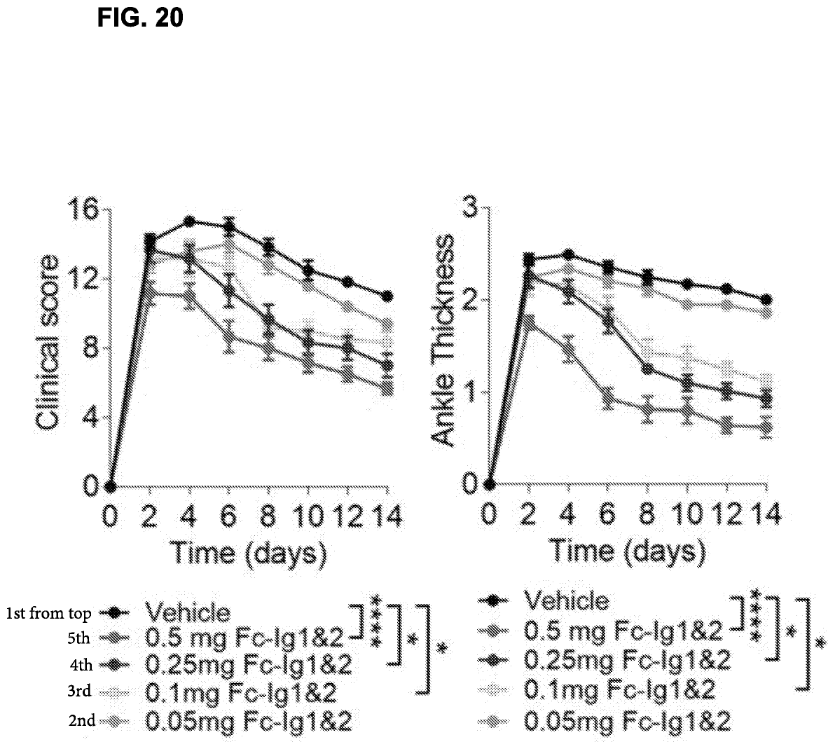

IL-6 down-regulates PTPRS expression in RA FLS. RA FLS (n=4 lines) were serum starved for 24h before stimulation with IL-6 (25 ng/ml) for 12h. Expression of PTPRS analyzed by qPCR and normalized to GAPDH. RQ calculated against unstimulated (Un) cells (FIG. 19).

BALB/c mice were injected with 100 microl of arthritogenic K/B×N serum to induce STIA and then injected i.p. with the indicated amounts of Fc-Ig1&2 every other day beginning on day 0. Arthritis was scored every two days for two weeks. Left panel shows clinical scoring and right panel shows ankle thickness as measured by caliper. Means+−s.e.m. are shown. *p<0.05, **** p<0.0001. Differences were measured by ANOVA. These experiments demonstrate the effect of Fc-Ig1&2 on arthritis scores.

1. A pharmaceutical composition comprising a first amount of a PTPRS de-clustering agent and a second amount of a TNF inhibitor or an IL-6 inhibitor, wherein the second amount is below a therapeutically effective level of the TNF inhibitor.

2. The pharmaceutical composition of claim 1, wherein said second amount is at least 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99% below a therapeutically effective level of the TNF inhibitor or IL-6 inhibitor.

3. The pharmaceutical composition of claim 1, wherein said therapeutically effective level of the TNF inhibitor or IL-6 inhibitor is measured by an at least 5%, 10%, 15%, 20%, 25%, 40%, 50%, 60%, 75%, 80%, 90%, or at least 100% or at least a 1.2-fold, 1.5-fold, 2-fold, 5-fold increase in (a) amelioration of disease or one or more symptoms of disease or (b) delay of onset of disease or one or more symptoms of disease.

4. The pharmaceutical composition of claim 1, wherein said TNF inhibitor comprises Etanercept, Adalimumab, Infliximab, Golimumab, Certolizumab or Certolizumab pegol, or a biosimilar thereof.

5. The pharmaceutical composition of claim 2, wherein the second amount comprises:
Etanercept or a biosimilar thereof, wherein the therapeutically effective level is 50 mg;
Adalimumab or a biosimilar thereof, wherein the therapeutically effective level is 40 mg;
Infliximab or a biosimilar thereof, wherein the therapeutically effective level is 3 mg/kg;
Golimumab or a biosimilar thereof, wherein the therapeutically effective level is 50 mg; or
Certolizumab or a biosimilar thereof, wherein the therapeutically effective level is 400 mg.

6-9. (canceled)

10. The pharmaceutical composition of claim 1, wherein said first amount is below a therapeutically effective level of the PTPRS de-clustering agent.

11-13. (canceled)

14. The pharmaceutical composition of claim 1, wherein said IL-6 inhibitor comprises Tocilizumab or Sarilumab.

15. The pharmaceutical composition of claim 1, wherein the second amount comprises:
Tocilizumab or a biosimilar thereof, wherein the therapeutically effective level is 4 mg/kg IV;
Tocilizumab or a biosimilar thereof, wherein the therapeutically effective level is 162 mg SC; or
Sarilumab or a biosimilar thereof, wherein the therapeutically effective level is 100 mg.

16-22. (canceled)

23. The pharmaceutical composition of claim 1, wherein the PTPRS de-clustering agent comprises one or both of PTPRS immunoglobulin-like domain 1 (Ig1) and immunoglobulin-like domain 2 (Ig2).

24. The pharmaceutical composition of claim 23, wherein the PTPRS de-clustering agent comprises at least about 60%, identity to PTPRS immunoglobulin-like domain 1 (Ig1) or immunoglobulin-like domain 2 (Ig2).

25. The pharmaceutical composition of claim 23, wherein the PTPRS de-clustering agent comprises Ig1&2.

26. The pharmaceutical composition of claim 25, wherein the PTPRS de-clustering agent comprises at least about 60%, identity to Ig1&2.

27. The pharmaceutical composition of claim 23, wherein the PTPRS de-clustering agent comprises Fc-Ig1&2.

28. The pharmaceutical composition of claim 27, wherein the PTPRS de-clustering agent comprises at least about 60%, identity to Fc-Ig1&2.

29. The pharmaceutical composition of claim 23, wherein the PTPRS de-clustering agent comprises Ig1 amino acid residues 30 to 127 of SEQ ID NO:4 or amino acid residues 30-127 of SEQ ID NO:8.

30. The pharmaceutical composition of claim 29, wherein the PTPRS de-clustering agent comprises an amino acid sequence that is at least 60% identical to the sequence set forth as:
(SEQ ID NO: 1)
EEPRFIKEPKDQIGVSGGVASFVCQATGDPKPRVTWNKKGKKVNSQRFE
TIEFDESAGAVLRIQPLRTPRDENVYECVAQNSVGEITVHAKLTVLRE
or
as set forth in SEQ ID NO: 5.

31. (canceled)

32. The pharmaceutical composition of claim 23, wherein the PTPRS de-clustering agent comprises an amino acid sequence that is at least 60% identical to residues 128 to 231 of SEQ ID NO:4 or residues 128-244 of SEQ ID NO:8.

33. (canceled)

34. The pharmaceutical composition of claim 32, wherein the PTPRS de-clustering agent comprises an amino acid sequence that is at least 60% identical to the sequence set forth as:
(SEQ ID NO: 2)
DQLPSGFPNIDMGPQLKVVERTRTATMLCAASGNPDPEITWFKDFLPVD
PSASNGRIKQLRSETFESTPIRGALQIESSEETDQGKYECVATNSAGVR
YSSPANLYVRVRRVA
or
as set forth in SEQ ID NO: 6.

35. (canceled)

36. The pharmaceutical composition of claim 1, wherein the PTPRS de-clustering agent comprises an Ig3 amino acid sequence that is at least 60% identical to residues 232-321 of SEQ ID NO:4 or residues 245-334 of SEQ ID NO:8.

37. (canceled)

38. The pharmaceutical composition of claim 36, wherein the PTPRS de-clustering agent comprises an amino acid sequence that is at least 60% identical to the sequence set forth as:
(SEQ ID NO: 3)
PRFSILPMSHEIMPGGNVNITCVAVGSPMPYVKWMQGAEDLTPEDDMPV
GRNVLELTDVKDSANYTCVAMSSLGVIEAVAQITVKSLPKA
or
as set forth in SEQ ID NO: 7.

39-42. (canceled)

43. A method of treating an autoimmune disease in a subject, the method comprising administering to the subject the pharmaceutical composition of claim 1.

44. (canceled)

45. The method of claim 43, wherein the autoimmune disease is arthritis, scleroderma, or Crohn's disease.

46. The method of claim 45, wherein the autoimmune disease is rheumatoid arthritis.

47. (canceled)

48. A method of decreasing fibroblast activity in a subject, the method comprising administering to the subject the pharmaceutical composition of claim 1.

49. (canceled)

50. The method of claim 48, wherein the fibroblast activity comprises fibroblast migration, collagen production, glycosaminoglycan production, reticular and elastic fiber production, cytokine production, chemokine production, glycoprotein production, extracellular matrix production or combinations thereof.

51-54. (canceled)

55. The method of claim 48, wherein the subject has a fibroblast-mediated disease.

56. The method of claim 55, wherein the fibroblast-mediated disease is fibrosis or a fibroblast-mediated autoimmune disease.

57. The method of claim 56, wherein the fibrosis is pulmonary fibrosis, idiopathic pulmonary fibrosis, liver fibrosis, endomyocardial fibrosis, atrial fibrosis, mediastinal fibrosis, myelofibrosis, retroperitoneal fibrosis, nephrogenic systemic fibrosis, skin fibrosis, or arthrofibrosis.

58. (canceled)

59. The method of claim 56, wherein the fibroblast-mediated autoimmune disease is selected from the group consisting of Crohn's disease, arthritis, rheumatoid arthritis, and scleroderma.

60. A method of modulating extracellular matrix in a subject, the method comprising administering to the subject an effective amount of the pharmaceutical composition of claim 1, wherein administration modulates the extracellular matrix in the subject.

61. The method of claim 60, wherein modulation of the extracellular matrix comprises modulation of one or more components of the extracellular matrix.

62. The method of claim 61, wherein the extracellular matrix component comprises a proteoglycan, polysaccharide, or fiber.

63. (canceled)

64. The method of claim 62, wherein the proteoglycan is heparan sulfate.

65. The method of claim 60, wherein the subject has an extracellular matrix disease.

66. The method of claim 65, wherein the extracellular matrix disease is selected from the group consisting of atherosclerosis, cancer, an amyloid disease, an inflammatory condition, and a developmental disorder.

67. The method of claim 66, wherein the amyloid disease is Alzheimer's disease or inflammation-related AA amyloidosis.

68. The method of claim 66, wherein the inflammatory condition is osteoarthritis, systemic scleroderma, or lupus.

69-74. (canceled)
